(19) United States
(12) Patent Application Publication
(10) Pub. No.: US 2022/0372166 A1
(43) Pub. Date: Nov. 24, 2022

(54) MEANS AND METHODS FOR TREATING SUBJECTS WITH HER2 AND HER3 POSITIVE CANCER

(71) Applicant: Merus N.V., Utrecht (NL)

(72) Inventors: Ernesto Isaac WASSERMAN, Utrecht (NL); Leonardo Andres SIRULNIK, Utrecht (NL)

(73) Assignee: Merus N.V., Utrecht (NL)

(21) Appl. No.: 17/755,196

(22) PCT Filed: Oct. 23, 2020
(86) PCT No.: PCT/NL2020/050656

§ 371 Date: Apr. 22, 2022

(30) Foreign Application Priority Data
Oct. 24, 2019 (NL) 2024097

(51) Int. Cl. C07K 16/32; A61P 35/02

(52) U.S. Cl. CPC C07K 16/32; A61P 35/02; A61K 2039/505

(57) ABSTRACT

The invention relates to a treatment using a bispecific antibody that comprises a first antigen-binding site that binds an extracellular part of ErbB-2 and a second antigen-binding site that binds an extracellular part of ErbB-3 for subjects that have cancer that has progressed after receiving a prior treatment. The prior treatment comprises a chemotherapy, a monospecific bivalent antibody comprising antigen-binding sites that bind an extracellular part of ErbB-2 or an extracellular part of ErbB-3, or a prior treatment with a tyrosine kinase inhibitor (TKI) of ErbB-2 or with a combination thereof.

The invention relates to the field of antibodies. In particular it relates to the field of therapeutic (human) antibodies for the treatment of a subject with an ErbB-2/ErbB-3 positive cancer. More in particular it relates to treating cancers comprising a neuregulin-1 (NRG1) fusion gene comprising at least a portion of the NRG1-gene fused to a sequence from a different chromosomal location.

Proteolytic processing of the extra cellular domain of the transmembrane NRG1 isoforms release soluble factors. HRG1-61 is one of the proteins encoded by the gene. It contains an Ig domain and an EGF-like domain that is necessary for direct binding to receptor tyrosine kinases ErbB-3 and ErbB-4. The NRG1 gene and the isoforms are known under a number of different aliases such as: Neuregulin 1; Pro-NRG1; HRGA; SMDF; HGL; GGF; NDF; NRG1 Intronic Transcript 2 (Non-Protein Coding); Heregulin, Alpha (45kD, ERBB2 P185-Activator); Acetylcholine Receptor-Inducing Activity; Pro-Neuregulin-1, Membrane-Bound Isoform; Sensory And Motor Neuron Derived Factor; Neu Differentiation Factor; Glial Growth Factor 2; NRG1-IT2; MSTP131; MST131; ARIA; GGF2; HRG1; and HRG. External Ids for NRG1 Gene are HGNC: 7997; Entrez Gene: 3084; Ensembl: ENSG00000157168; OMIM: 142445 and UniProtKB: Q02297. Isoforms of NRG1 are made by alternative splicing, and include forms that are transmembrane, externally membrane bound, shed, secreted or intracellular (Falls, 2003; Hayes and Gullick, 2008). They bind to ErbB-3 or ErbB-4, which is understood to signal heterodimer formation with ErbB-2 (HER2). Although the NRG1-encoded proteins are usually thought of as mitogens, they can also be powerfully proapoptotic: in particular, expressing NRG1 in cells can cause apoptosis of the expressing cell Weinstein et al. (1998). Oncogene 17: 2107-2113.

NRG1 gene fusions are understood to be oncogenic drivers. In PCT/NL2018/050206, the applicants have reported the capability to target and treat cancer harboring NRG1-fusions, including with a bispecific antibody targeting ErbB-2 and ErbB-3. Recently, clinical responses to tyrosine kinase inhibitors (TKI) and mAbs have been reported (Drilon A et al (2018), Response to ERBB3-directed targeted therapy in NRG1-rearranged cancers. Cancer Discov Vol 8:686-95. Gay ND et al (2017), Durable response to afatinib in lung adenocarcinoma harboring NRG1 gene fusions. J Thorac Oncol 12:e107-10. Jones MR et al (2017), Successful targeting of the NRG1 pathway indicates novel treatment strategy for metastatic cancer. Ann Oncol Vol .28: 3092-7; and Cheema PK et al (2017), A case of invasive mucinous pulmonary adenocarcinoma with a CD74-NRG1 fusion protein targeted with afatinib. J Thorac Oncol 12:e200-2). Targeting of the signaling axis can be directed to various stages and parts of the signaling pathway. Among these are therapies directed towards ErbB-2, ErbB-3 or both. Medicaments can be directed towards the extra-cellular parts of the molecules and/or to intracellular parts. Antibodies such as trastuzumab bind to and inhibit signaling of ErbB-2. Therapeutic ErbB-3 directed antibodies have also been described.

With respect to the above clinical reports, while certain level of efficacy has been reported ranging from stable disease to partial response with limited durability, it is observed that tumors may progress breaking durability, recur or that metastasis of the original tumor may be detected. Such recurrences, metastasis have often become more resistant to treatment with the medicament. In the present invention, the inventors have identified that recurrence, progression or metastasis after treatment of the tumor with a TKI, chemotherapy, anti-ErbB-2 or anti-ErbB-3 targeting molecule can be successfully treated with a bispecific antibody that comprises a first antigen-binding site that binds an extracellular part of ErbB-2 and a second antigen-binding site that binds an extracellular part of ErbB-3.

### SUMMARY OF THE INVENTION

The invention provides a bispecific antibody that comprises a first antigen-binding site that binds an extracellular part of ErbB-2 and a second antigen-binding site that binds an extracellular part of ErbB-3 for use in the treatment of a subject that has an ErbB-2 and ErbB-3 positive cancer, cells of which cancer comprise a neuregulin-1 (NRG1) fusion gene comprising at least a portion of the NRG1-gene fused to a sequence from a different chromosomal location, and which cancer in said subject has progressed after having received a prior treatment with chemotherapy, a monospecific bivalent antibody comprising antigen-binding sites that bind an extracellular part of ErbB-2 or an extracellular part of ErbB-3, or a prior treatment with a tyrosine kinase inhibitor (TKI) of ErbB-2 or with a combination thereof.

The subject is preferably a human subject.

The chemotherapy according to the present invention preferably comprises gemcitabine, capecitabine, carboplatin, a taxane, such as docetaxel or paclitaxel, 5-fluorouracil (with or without radiotherapy), vinorelbine, mitoxantrone, vinblastine, cisplatin (or pemetrexed), oxaliplatin, carboplatin, ifosfamide, mytomycine C, vindesine, etoposide, Folfox (i.e. a combination of 5-fluorouracil, leucovorin, and oxaliplatin) or Folfiri (i.e. a combination of leucovorin, 5-fluorouracil and irinotecan),Folfirinox (a combination of leucovorin, 5-fluorouracil, irinotecan and oxaliplatin) or any combination thereof.

The monospecific bivalent antibody comprising antigen-binding sites that bind an extracellular part of ErbB-2 is preferably trastuzumab, pertuzumab, or trastuzumab-emtansine. The ErbB-2 TKI is preferably one or more of lapatinib, canertinib, neratinib, tucatinib (irbinitinib), CP-724714, tarloxitinib, mubritinib, afatinib, varlitinib, and dacomitinib, preferably afatinib.

In one embodiment the ErbB-2 TKI is afatinib.

The monospecific bivalent antibody comprising antigen-binding sites that bind an extracellular part of ErbB-3 preferably comprises patritumab, seribantumab, lumretuzumab, elgemtumab, GSK2849330, KTN3379 or AV-203.

The prior treatment that the cancer has progressed from preferably comprises chemotherapy, a TKI, an ErbB-2 or an ErB-3 targeted tumor treatment as indicated herein above. The ErbB-2 targeted treatment is preferably with a monospecific bivalent antibody comprising antigen-binding sites that bind an extracellular part of ErbB-2. Such antibody is preferably trastuzumab, pertuzumab, or trastuzumab-emtansine. The ErbB-2 targeted treatment is preferably an ErbB-2 TKI. The ErbB-2 TKI is preferably one or more of lapatinib, canertinib, neratinib, tucatinib (or irbinitinib), CP-724714, tarloxitinib, mubritinib, afatinib, varlitinib, and dacomitinib, preferably afatinib. An ErbB-2 TKI may also affect ErbB-1 signaling but is different from an ErbB-1 TKI in that it has significant activity on ErbB-2. The ErbB-3 targeted treatment is preferably with a monospecific bivalent antibody comprising antigen-binding sites that bind an extracellular part of ErbB-3. Such antibody is preferably patritumab, seribantumab, lumretuzumab, elgemtumab, GSK2849330, KTN3379 or AV-203.

A target specific treatment may be combined with other treatments for the same target. Alternatively a treatment that targets one target may also be active on another of the mentioned targets.

The NRG1 fusion gene preferably comprises at least the 3′ end of the NRG1-gene fused to a 5′ sequence from a different chromosomal location. When present in a cancer cell the cancer cell is preferably driven by the NRG1-fusion gene. The NRG1-fusion gene preferably expresses a protein that comprises an NRG1 EGF- like domain. In some embodiments the NRG1-fusion gene is a fusion of NRG1 and a gene on human chromosome 8. The gene on human chromosome 8 preferably encodes an excreted protein or a cellular membrane associated protein. In some embodiments the NRG1 fusion gene is a fusion of the 3′ end of the NRG1-gene with the 5′ sequence of one of the genes selected from the group consisting of ADAM9, AKAP13, APP, ATP1B1, BMPR1B, CCND1, CD44, CD74, CDH1, CDH6, CDK1, CLU, COX10-AS1, DIP2B, DOC4, DPYSL2, FOXA1, GDF15, HMBOX1, KIF13B, MCPH1, MDK, MRPL13, NOTCH2, PARP8, PDE7A, POMK, RAB2IL1, RAB3IL1, RBPMS, ROCK1, SDC4, SETD4, SLC3A2, SLC4A4, SMAD4, STAU3, THAP7, THBS1, TNC, TNFRSF10B, TNKS, TSHZ2, VAMP2, VTCN1, WHSC1L1, WRN and ZMYM2, preferably an SDC4; ATP1B1; or a CD74 fusion, more preferably an SDC4-NRG1 fusion. In a preferred embodiment the NRG1 fusion gene is a fusion of the 3′ end of the NRG1-gene with the 5′ sequence of the gene SDC4. In several cases, the NRG1 fusion gene is a fusion of the 5′ end of the NRG1-gene with the 3′ sequence of the fusion partner. In a preferred embodiment, the 5′NRG1 fusion involves genes selected from the group consisting of FOXA1, PMEPA1, RAD51, and STMN2. In all these cases, it is preferred that the gene fusion protein product is translated into a protein that comprises an NRG1 EGF-like domain.

The cancer is preferably a recurrent cancer or a metastasized cancer. Recurrence typically refers to local recurrence and means that the cancer is in the same place as the original cancer or very close to it. A tumor is typically said to be a metastasized tumor when the tumor has migrated to lymph nodes or tissues near the original cancer or spread to more distant organs or tissues far from the original cancer. The difference between recurrence and metastasis is not always very clear when at more or less at the same position as the original tumor. In such cases both indications can be used.

The cancer is preferably a pancreatic cancer, pancreatic ductal adenocarcinoma, sarcoma, bladder, colorectal, gallbladder, head and neck cancer, prostrate, uterus, breast cancer, an ovarian cancer, a liver cancer, an endometrial cancer, a lung cancer such as non-small cell lung cancer, preferably a non-small cell lung cancer, more preferably invasive mucinous adenocarcinoma. Preferably, the cancer is pancreatic ductal adenocarcinoma and the tumor genome shows absence of mutations in one or more genes selected from the group consisting of EGFR, KRAS, cKIT-BRCA1-2, MET, ROS, RET, ALK, preferably KRAS. Thus, preferably, the cancer is pancreatic ductal adenocarcinoma and the tumor genome is KRAS wildtype.

The bispecific antibody that comprises a first antigen-binding site that binds an extracellular part of ErbB-2 and a second antigen-binding site that binds an extracellular part of ErbB-3 preferably has a first antigen-binding site that binds domain I of ErbB-2 and a second antigen-binding site that binds domain III of ErbB-3. In some embodiments the affinity of the first antigen-binding site for ErbB-2 is lower than the affinity of the second antigen-binding site for ErbB-3.

The bispecific antibody preferably comprises

i) at least the CDR1, CDR2 and CDR3 sequences of an ErbB 2 specific heavy chain variable region selected from the group consisting of MF2926, MF2930, MF1849; MF2973, MF3004, MF3958, MF2971, MF3025, MF2916, MF3991, MF3031, MF2889, MF2913, MF1847, MF3001, MF3003 and MF1898 or wherein said antibody comprises CDR sequences that differ in at most 3 amino acids, preferably in at most 2 amino acids, preferably in at most 1 amino acid from the CDR1, CDR2 and CDR3 sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) at least the CDR1, CDR2 and CDR3 sequences of an ErbB 3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

In one embodiment the bispecific antibody comprises

i) an ErbB 2 specific heavy chain variable region sequence selected from the group consisting of the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 and MF1898, or wherein said antibody comprises a heavy chain variable region sequence that differs in at most 15 amino acids from the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) an ErbB 3 specific heavy chain variable region sequence selected from the group consisting of the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

In a preferred embodiment the ErbB-2 binding variable domain of the bispecific antibody comprises at least the CDR1, CDR2 and CDR3 sequences of the ErbB 2 specific heavy chain variable region MF3958 and the ErbB-3 binding variable domain of the bispecific antibody at least comprises the CDR1, CDR2 and CDR3 sequences of the ErbB 3 specific heavy chain variable region MF3178.

The variable domain that comprises said first antigen binding site and the variable domain that comprises said second antigen binding site of said bispecific antibody preferably comprise a light chain variable region comprising the IgVK1-39 gene segment, most preferably the rearranged germline human kappa light chain IgVK1-39*01/IGJK1*01.

The variable domain that comprises said first antigen binding site and the variable domain that comprises said second antigen binding site of said bispecific antibody preferably comprise a light chain variable region comprising a CDR1 having the sequence (RASQSISSYLN), a CDR2 having the sequence (AASSLQS), and a CDR3 having the sequence (QQSYSTPPT), according to KABAT numbering or according to the IMGT numbering system, the CDRs are QSISSY, AAS and QQSYSTPPT, respectively.

The variable domain that comprises said first antigen binding site and the variable domain that comprises said second antigen binding site of said bispecific antibody preferably comprise a light chain variable region of FIG. 1a or FIG. b.

The invention further provides a method of treating a subject that has an ErbB-2 and ErbB-3 positive cancer, cells of which cancer comprise an NRG1 fusion gene comprising at least a portion of the NRG1-gene fused to a sequence from a different chromosomal location, and which subject is a pretreatment cancer subject that had received a previous chemotherapy, or ErbB-2 or ErbB-3 targeted tumor treatment, the method comprising administering to the subject in need thereof a bispecific antibody that comprises a first antigen-binding site that binds an extracellular part of ErbB-2 and a second antigen-binding site that binds an extracellular part of ErbB-3.

### DETAILED DESCRIPTION OF THE INVENTION

An NRG1 fusion gene comprises at least a portion of the NRG1-gene fused to a sequence from a different chromosomal location. “At least a portion” indicates that the entire NRG-1 gene may be present in a fusion or a portion thereof. The fusion preferably has at least the coding sequence of exons 6, 7 and 8. Preferably, the NRG1 part in the NRG1-fusion gene comprises the EGF-like domain of NRG1. The at least a portion of the NRG1 gene may be fused to a sequence from a different chromosomal location such that the said sequence is located 5′ or 3′to the at least a portion of the NRG1 gene.

Preferably, the 3′ end of the NRG1-gene may be fused to a 5′ sequence from a different chromosomal location. The NRG1-gene codes for the various isoforms of NRG1. Various isoforms and their expected function are described in Adelaide et al (2003). GGF and GGF2 isoforms contain a kringle-like sequence plus Ig and EGF-like domains; and the SMDF isoform shares only the EGF-like domain with other isoforms. The EGF-like domain is encoded by the 3′ end of the gene. The EGF-like domain is present in all NRG1 fusion genes of the present invention. Fusions have been found wherein the 5′ from different chromosomal location comprises an excretion signal and/or or a transmembrane domain of a cellular membrane protein with at least one extracellular domain. An example is the CD74-NRG1 fusion. The 5′ sequence from different chromosomal location may also insert a sequence that activates transcription of NRG1, examples are promoters or enhancers. The 5′ sequence is typically a sequence from a gene different than NRG1. The sequence can comprise a coding region, an expression regulatory sequence such as a promoter or an enhancer, or a combination thereof. The NRG-fusion comprises a 5′ sequence from a different location can be from a different chromosome or from another part of chromosome 8. In a preferred embodiment the 5′ sequence is from a gene on human chromosome 8.

The NRG1-gene, for example the 3′ end of the NRG-1 gene, in the fusion preferably has at least the coding sequence of exons 6, 7 and 8. Another way to define the NRG1 part in the NRG1-fusion gene is that it comprises the EGF-like domain of NRG1. This domain is encoded by the 3′ end of the NRG1 gene (exons 6-8) and is necessary for binding to ErbB-3. NRG1-fusions retain an in-frame coding region for this EGF-like domain at the 3′ end of the fusion. An EGF-like domain is a sequence of typically about thirty to forty amino-acid residues long of which the prototype is found in the sequence of epidermal growth factor (EGF) [PMID: 2288911, PMID: 6334307, PMID: 1522591, PMID: 6607417, PMID: 3282918, PMID: 11498013]. It is known to be present, in a more or less conserved form, in a large number of other, mostly animal proteins. A common feature of EGF-like domains is that they are found in the extracellular domain of membrane-bound proteins or in proteins known to be secreted (exception: prostaglandin G/H synthase).

The NRG1 fusion gene is preferably a fusion of the 3′ end of the NRG1-gene with the 5′ sequence of one of the genes selected from the group consisting of ADAM9, AKAP13, APP, ATP1B1, BMPR1B, CCND1, CD44, CD74, CDH1, CDH6, CDK1, CLU, COX10-AS1, DIP2B, DOC4, DPYSL2, FOXA1, GDF15, HMBOX1, KIF13B, MCPH1, MDK, MRPL13, NOTCH2, PARP8, PDE7A, POMK, RAB2IL1, RAB3IL1, RBPMS, ROCK1, SDC4, SETD4, SLC3A2, SLC4A4, SMAD4, STAU3, THAP7, THBS1, TNC, TNFRSF10B, TNKS, TSHZ2, VAMP2, VTCN1, WHSC1L1, WRN and ZMYM2, preferably an SDC4, ATP1B1 or a CD74 fusion, more preferably an SDC4-NRG1 fusion. In a preferred embodiment the NRG1 fusion gene is a fusion of the 3′ end of the NRG1-gene with the 5′ sequence of the gene SDC4. In several cases, the NRG1 fusion gene is a fusion of the 5′ end of the NRG1-gene with the 3′ sequence of the fusion partner. In a preferred embodiment, the 5′NRG1 fusion involves genes selected from the group consisting of FOXA1, PMEPA1, RAD51, and STMN2. In all these cases, it is preferred that the gene fusion protein product is translated into a protein that comprises an NRG1 EGF-like domain.

The NRG1 fusion gene is preferably a fusion of the 3′ end of the NRG1-gene with the 5′ sequence of the SDC4 gene.

The NRG1 fusion gene may be a fusion of at least a portion of the NRG1-gene with a sequence from a different chromosomal location located 3′ thereto. Such a NRG1 fusion gene may be a fusion of at least a portion of the NRG1-gene with a sequence from a different chromosomal location CD74, STMN2, PMEPA1, PROSC or PSAP located 3′ thereto.

The receptors for all NRG1 isoforms are the ErbB family of tyrosine kinase transmembrane receptors. The family is also referred to as the human epidermal growth factor (EGF) receptor family (HER). The family has four members: ErbB (Erythroblastoma)-1, ErbB-2, ErbB-3 and ErbB-4. The receptors (reviewed in Yarden and Pines 2012) are widely expressed on epithelial cells. Upregulation of HER receptors or their ligands, such as heregulin (HRG) or epidermal growth factor (EGF), is a frequent event in human cancer (Wilson, Fridlyand et al. 2012). Overexpression of ErbB-1 and ErbB-2 in particular occurs in epithelial tumors and is associated with tumor invasion, metastasis, resistance to chemotherapy, and poor prognosis (Zhang, Berezov et al. 2007). In the normal breast, ErbB-3 has been shown to be important in the growth and differentiation of luminal epithelium. For instance, loss/inhibition of ErbB-3 results in selective expansion of the basal over the luminal epithelium (Balko, Miller et al. 2012). Binding of ligand to the extracellular domain of the RTKs induces receptor dimerization, both between the same (homodimerization) and different (heterodimerization) receptor subtypes. Dimerization can activate the intracellular tyrosine kinase domains, which undergo autophosphorylation and, in turn, can activate a number of downstream pro-proliferative signaling pathways, including those mediated by mitogen-activated protein kinases (MAPK) and the prosurvival pathway Akt (reviewed in Yarden and Pines, 2012). No specific endogenous ligand has been identified for ErbB-2, which is therefore assumed to normally signal through heterodimerization (Sergina, Rausch et al. 2007). ErbB-3 can be activated by engagement of its ligands. These ligands include but are not limited to neuregulin (NRG) and heregulin (HRG).

ErbB-1 is known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This receptor tyrosine kinase (RTK) has been extensively used as target for cancer therapy. Both small-molecule inhibitors targeting the RTK and monoclonal antibodies (mAbs) (monospecific bivalent) directed to the extracellular ligand-binding domains have been developed and have shown hitherto several clinical successes. The database accession number for the human EGFR protein and the gene encoding it is (GenBank NM_005228.3). This accession number is primarily given to provide a further method of identification of EGFR protein as a target, the actual sequence of the EGFR protein bound by an antibody may vary, for instance because of a mutation in the encoding gene such as those occurring in some cancers or the like.

The term ‘ErbB-2’ as used herein refers to the protein that in humans is encoded by the ERBB-2 gene. Alternative names for the gene or protein include CD340; HER-2; HER-2/neu; MLN 19; NEU; NGL; TKR1. The ERBB-2 gene is frequently called HER2 (from human epidermal growth factor receptor 2). Where reference is made herein to ErbB-2, the reference refers to human ErbB-2. An antibody comprising an antigen-binding site that binds ErbB-2, binds human ErbB-2. The ErbB-2 antigen-binding site may, due to sequence and tertiary structure similarity between human and other mammalian orthologs, also bind such an ortholog but not necessarily so. Database accession numbers for the human ErbB-2 protein and the gene encoding it are (NP_001005862.1, NP_004439.2 NC_000017.10 NT_010783.15 NC_018928.2). The accession numbers are primarily given to provide a further method of identification of ErbB-2 as a target, the actual sequence of the ErbB-2 protein bound the antibody may vary, for instance because of a mutation in the encoding gene such as those occurring in some cancers or the like. The ErbB-2 antigen binding site binds ErbB-2 and a variety of variants thereof, such as those expressed by some ErbB-2 positive tumor cells. The antigen-binding site that binds ErbB-2 preferably binds domain I of ErbB-2.

The term ‘ErbB-3’ as used herein refers to the protein that in humans is encoded by the ERBB3 gene. Alternative names for the gene or protein are HER3; LCCS2; MDA-BF-1; c-ErbB-3; c-ErbB3; ErbB3-S; p180-ErbB3; p45-sErbB3; and p85-sErbB3. Where reference is made herein to ErbB-3, the reference refers to human ErbB-3. An antibody comprising an antigen-binding site that binds ErbB-3, binds human ErbB-3. The ErbB-3 antigen-binding site may, due to sequence and tertiary structure similarity between human and other mammalian orthologs, also bind such an ortholog but not necessarily so. Database accession numbers for the human ErbB-3 protein and the gene encoding it are (NP_001005915.1, NP_001973.2, NC_000012.11, NC_018923.2, NT_029419.12). The accession numbers are primarily given to provide a further method of identification of ErbB-3 as a target, the actual sequence of the ErbB-3 protein bound by an antibody may vary, for instance because of a mutation in the encoding gene such as those occurring in some cancers or the like. The ErbB-3 antigen binding site binds ErbB-3 and a variety of variants thereof, such as those expressed by some ErbB-3 positive tumor cells. The antigen-binding site that binds ErbB-3 preferably binds domain III of ErbB-3.

When reference is made to ErbB-1, ErbB-2 or ErbB-3 or an alternative name for the same, the reference is to human ErbB-1, ErbB-2 or ErbB-3. Antibodies as referred to herein bind to ErbB-1, ErbB-2 or ErbB-3 and many mutated ErbB-1, ErbB-2 or ErbB-3 proteins as can be found in cancers.

CD74 is known under number of aliases. Some of these are CD74 Molecule; CD74 Antigen (Invariant Polypeptide Of Major Histocompatibility Complex, Class II Antigen-Associated); CD74 Molecule, Major Histocompatibility Complex, Class II Invariant Chain; HLA-DR Antigens-Associated Invariant Chain; Gamma Chain Of Class II Antigens; Ia-Associated Invariant Chain; MHC HLA-DR Gamma Chain; HLA-DR-Gamma; DHLAG; P33; HLA Class II Histocompatibility Antigen Gamma Chain; Ia Antigen-Associated Invariant Chain; Ia-GAMMA and HLADG. External Ids for CD74 are HGNC: 1697; Entrez Gene: 972; Ensembl: ENSG00000019582; OMIM: 142790 and UniProtKB: P04233.

DOC4 or Teneurin Transmembrane Protein 4 (TENM4) is known under a number of different names such as Protein Odd Oz/Ten-M Homolog 4; Tenascin-M4; Ten-M4; Ten-4; ODZ4; TNM4; Odz, Odd Oz/Ten-M Homolog 4 (Drosophila); Odz, Odd Oz/Ten-M Homolog 4; Teneurin-4; KIAA1302; Doc4; and ETMS. External Ids for DOC4 are HGNC: 29945; Entrez Gene: 26011; Ensembl: ENSG00000149256; OMIM: 610084 and UniProtKB: Q6N022.

TNFRSF1OB or TNF Receptor Superfamily Member 10b is known under a number of different names Tumor Necrosis Factor Receptor Superfamily, Member 10b; TNF-Related Apoptosis-Inducing Ligand Receptor 2; Death Receptor 5; TRAIL-R2; TRAILR2; KILLER; TRICK2; ZTNFR9; DRS; P53-Regulated DNA Damage-Inducible Cell Death Receptor(Killer); Tumor Necrosis Factor Receptor Superfamily Member 10B; Tumor Necrosis Factor Receptor-Like Protein ZTNFR9; Death Domain Containing Receptor For TRAIL/Apo-2L; poptosis Inducing Protein TRICK2A/2B; Apoptosis Inducing Receptor TRAIL-R2; Cytotoxic TRAIL Receptor-2; Fas-Like Protein; TRAIL Receptor 2; CD262 Antigen; KILLER/DRS; TRICK2A; TRICK2B; TRICKB; and CD262. External Ids for TNFRSF1OB are HGNC: 11905; Entrez Gene: 8795; Ensembl: ENSG00000120889; OMIM: 603612; and UniProtKB: 014763.

The CLU gene or Clusterin is known under a number of different names such as Testosterone-Repressed Prostate Message 2; Apolipoprotein J; Complement-Associated Protein SP-40,40; Complement Cytolysis Inhibitor; Complement Lysis Inhibitor; Sulfated Glycoprotein 2; Ku70-Binding Protein 1; NAl/NA2; TRPM-2; APO-J; APOJ; KUB1; CLI; Clusterin (Complement Lysis Inhibitor, SP-40,40, Sulfated Glycoprotein 2, Testosterone-Repressed Prostate

Message 2, Apolipoprotein J); Aging-Associated Gene 4 Protein; Aging-Associated Protein 4; SGP-2; SP-40; TRPM2; AAG4; CLU1; CLU2; and SGP2.External Ids for CLU are HGNC: 2095; Entrez Gene: 1191; Ensembl: ENSG00000120885; OMIM: 185430; and UniProtKB: P10909.

VAMP2 or Vesicle Associated Membrane Protein 2 is known under a number of different names such as synaptobrevin 2; SYB2; Vesicle-Associated Membrane Protein 2; and Synaptobrevin-2. External Ids for VAMP2 are HGNC: 12643; Entrez Gene: 6844; Ensembl: ENSG00000220205; OMIM: 185881; and UniProtKB: P63027.

SLCA3A2 or Solute Carrier Family 3 Member 2 is known under a number of different names such as Lymphocyte Activation Antigen 4F2 Large Subunit; Solute Carrier Family 3 (Activators Of Dibasic And Neutral Amino Acid Transport), Member 2; Antigen Identified By Monoclonal Antibodies 4F2, TRA1.10, TROP4,

And T43; Solute Carrier Family 3 (Amino Acid Transporter Heavy Chain), Member 2; 4F2 Cell-Surface Antigen Heavy Chain; CD98 Heavy Chain; 4F2HC; MDU1; Antigen Defined By Monoclonal Antibody 4F2, Heavy Chain; Antigen Defined By Monoclonal Antibody 4F2; 4F2 Heavy Chain Antigen; 4F2 Heavy Chain; CD98 Antigen; CD98HC; 4T2HC; NACAE; CD98 and 4F2. External Ids for SLC3A2 are

HGNC: 11026; Entrez Gene: 6520; Ensembl: ENSG00000168003; OMIM: 158070; and UniProtKB: P08195.

RBPMS or RNA Binding Protein With Multiple Splicing is known under a number of different names such as RNA Binding Protein With Multiple Splicing;

Heart And RRM Expressed Sequence; HERMES; RNA-Binding Protein With Multiple Splicing; and RBP-MS. External Ids for RBPMS are HGNC: 19097; Entrez Gene: 11030; Ensembl: ENSG00000157110; OMIM: 601558; and UniProtKB: Q93062.

WRN or Werner Syndrome RecQ Like Helicase is known under a number of different names such as Werner Syndrome RecQ Like Helicase; DNA Helicase, RecQ-Like Type 3; RecQ Protein-Like 2; Exonuclease WRN; RECQL2; RECQ3; Werner Syndrome ATP-Dependent Helicase; Werner Syndrome, RecQ Helicase-Like; Werner Syndrome; EC 3.6.4.12; EC 3.1.-.-; EC 3.6.1; and RECQL3. External Ids for WRN are HGNC: 12791; Entrez Gene: 7486; Ensembl: ENSG00000165392; OMIM: 604611 and UniProtKB: Q14191.

SDC4 or Syndecan 4 is known under a number of different names such as Syndecan 4 (Amphiglycan, Ryudocan); Syndecan Proteoglycan 4; Ryudocan Core Protein; Amphiglycan; SYND4; Ryudocan Amphiglycan; and Syndecan-4. External Ids for SDC4 are HGNC: 10661; Entrez Gene: 6385; Ensembl: ENSG00000124145; OMIM: 600017; and UniProtKB: P31431.

ADAM9 or A Disintegrin And Metalloproteinase Domain 9 (Meltrin Gamma) is known under a number of different names such as ADAM Metallopeptidase Domain; Disintegrin And Metalloproteinase Domain-Containing Protein 9; Metalloprotease/Disintegrin/Cysteine-Rich Protein 9; Cellular Disintegrin-Related Protein; Myeloma Cell Metalloproteinase; Cone Rod Dystrophy 9; MCMP; MDC9; ADAM Metallopeptidase Domain 9 (Meltrin Gamma); Meltrin-Gamma; Meltrin Gamma; EC 3.4.24.; EC 3.4.24; KIAA0021; CORD9; MLTNG. External Ids for ADAM9 Gene

### HGNC: 216; Entrez Gene: 8754; Ensembl: ENSG00000168615; OMIM: 602713; and UniProtKB: Q13443.

AKAP13 or A-Kinase Anchoring Protein 13 is known under a number of different names such as Lymphoid Blast Crisis Oncogene; Breast Cancer Nuclear Receptor-Binding Auxiliary Protein; Non-Oncogenic Rho GTPase-Specific GTP Exchange Factor; Guanine Nucleotide Exchange Factor Lbc; Protein Kinase A-Anchoring Protein 13; Human Thyroid-Anchoring Protein 31; A Kinase (PRKA) Anchor Protein 13; A-Kinase Anchor Protein 13; LBC Oncogene; AKAP-Lbc; AKAP-13; PRKA13; LBC; BRX; P47; PROTO-LBC; ARHGEF13; PROTO-LB; C-Lbc; HA-3; Ht31; and HT31. External Ids for AKAP13 Gene are HGNC: 371; Entrez Gene: 11214; Ensembl: ENSG00000170776; OMIM: 604686; and UniProtKB: Q12802.

APP or Amyloid Beta Precursor Protein is known under a number of different names such as Amyloid Beta (A4) Precursor Protein; Alzheimer Disease Amyloid Protein; Cerebral Vascular Amyloid Peptide; Amyloid-Beta Precursor Protein; Amyloid Precursor Protein; Amyloid-Beta A4 Protein; Peptidase Nexin-II; Protease Nexin-II; PreA4; PN-II; ABPP; APPI; CVAP; AD1; Beta-Amyloid Precursor Protein; Testicular Tissue Protein Li; Beta-Amyloid Peptide(1-40); Beta-Amyloid Peptide(1-42); Amyloid Beta A4 Protein; Beta-Amyloid Peptide; Alzheimer Disease; CTFgamma; ABETA; AAA; PN2; and A4. External Ids for APP Gene are HGNC: 620; Entrez Gene: 351; Ensembl: ENSG00000142192; OMIM: 104760; and UniProtKB: P05067.

ATP1B1 is known under a number of different names such as ATPase Na+/K+ Transporting Subunit Beta 1; Sodium/Potassium-Transporting ATPase Subunit Beta-1; Sodium-Potassium ATPase Subunit Beta 1 (Non-Catalytic); ATPase, Na+/K+ Transporting, Beta 1 Polypeptide; Sodium Pump Subunit Beta-1; ATP1B; Sodium/Potassium-Transporting ATPase Beta-1 Chain; Sodium/Potassium-Dependent ATPase Beta-1 Subunit; Sodium/Potassium-Dependent ATPase Subunit Beta-1; Beta 1-Subunit Of Na(+),K(+)-ATPase; Na, K-ATPase Beta-1 Polypeptide; and Adenosinetriphosphatase. External Ids for ATP1B1 Gene are HGNC: 804; Entrez Gene: 481; Ensembl: ENSG00000143153; OMIM: 182330; and UniProtKB: P05026.

BMPR1B is known under a number of different names such as Bone Morphogenetic Protein Receptor Type 1B; Bone Morphogenetic Protein Receptor, Type IB; Bone Morphogenetic Protein Receptor Type-1B; BMP Type-1B Receptor; EC 2.7.11.30; BMPR-1B; Serine/Threonine Receptor Kinase; Activin Receptor-Like Kinase 6; CDw293 Antigen; EC 2.7.11; CDw293; ALK-6; BDA1D; ALK6; AMDD; and BDA2. External Ids for BMPR1B Gene are HGNC: 1077; Entrez Gene: 658; Ensembl: ENSG00000138696; OMIM: 603248; and UniProtKB: 000238.

CCND1 or Cyclin D1 is known under a number of different names such as B-Cell Lymphoma 1 Protein; Gl/S-Specific Cyclin-D1; B-Cell CLL/Lymphoma 1; BCL-1 Oncogene; PRAD1 Oncogene; PRAD1; BCL1; Cyclin D1 (PRAD1: Parathyroid Adenomatosis 1); Parathyroid Adenomatosis 1; Gl/S-Specific Cyclin D1; D11S287E; U21B31; and BCL-1. External Ids for CCND1 Gene are HGNC: 1582; Entrez Gene: 595; Ensembl: ENSG00000110092; OMIM: 168461; and UniProtKB: P24385.

CD44 or is known under a number of different names such as CD44 Molecule (Indian Blood Group); Hematopoietic Cell E- And L-Selectin Ligand; GP90 Lymphocyte Homing/Adhesion Receptor; Chondroitin Sulfate Proteoglycan 8; Extracellular Matrix Receptor III; Heparan Sulfate Proteoglycan; Phagocytic Glycoprotein 1; Hyaluronate Receptor; Hermes Antigen; CD44 Antigen; ECMR-III; HUTCH-I; Epican; MDU2; MDU3; MIC4; LHR; CD44 Antigen (Homing Function And Indian Blood Group System); Homing Function And Indian Blood Group System; Cell Surface Glycoprotein CD44; Indian Blood Group Antigen; Phagocytic Glycoprotein I; Soluble CD44; CDW44; CSPG8; HCELL; CDw44; PGP-1; MC56; Pgpl; and IN. External Ids for CD44 Gene are HGNC: 1681; Entrez Gene: 960; Ensembl: ENSG00000026508; OMIM: 107269; and UniProtKB: P16070.

CDH1 or Cadherin 1 is known under a number of different names such as Cadherin 1, Type 1, E-Cadherin (Epithelial) ; Epithelial Cadherin; Cadherin-1; Uvomorulin; E-Cadherin; CAM 120/80; CDHE; UVO; Calcium-Dependent Adhesion Protein, Epithelial; Epididymis Secretory Sperm Binding Protein; Cadherin 1, E-Cadherin (Epithelial); Cell-CAM 120/80; CD324 Antigen; E-Cadherin 1; Arc-1; BCDS1; CD324; ECAD; and LCAM. External Ids for CDH1 Gene are HGNC: 1748; Entrez Gene: 999; Ensembl: ENSG00000039068; OMIM: 192090; and UniProtKB: P12830.

CDH6 or Cadherin 6 is known under a number of different names such as Cadherin 6, Type 2, K-Cadherin (Fetal Kidney); Cadherin-6; K-Cadherin; Kidney Cadherin; CAD6; and KCAD. External Ids for CDH6 Gene are HGNC: 1765; Entrez Gene: 1004; Ensembl: ENSG00000113361; OMIM: 603007; and UniProtKB: P55285.

CDK1 or Cyclin Dependent Kinase 1 is known under a number of different names such as Cell Division Cycle 2, G1 To S And G2 To M; Cell Division Control Protein 2 Homolog ; Cell Division Protein Kinase 1 ; Cyclin-Dependent Kinase 1 ; P34 Protein Kinase ; P34CDC2 ; CDC28A ; CDC2 ; Cell Cycle Controller CDC2; EC 2.7.11.22 ; EC 2.7.11.23; and CDKN1. External Ids for CDK1 Gene are HGNC: 1722; Entrez Gene: 983; Ensembl: ENSG00000170312; OMIM: 116940; and UniProtKB: P06493.

COX10-AS1 or COX10 Antisense RNA 1 is known under a number of different names such as COX10 Antisense RNA 1 (Non-Protein Coding); COX10 Antisense RNA (Non-Protein Coding); COX10-AS; and COX1OAS. External Ids for COX10-AS1 Gene are HGNC: 38873; Entrez Gene: 100874058; and Ensembl: ENSG00000236088.

DIP2B or Disco Interacting Protein 2 Homolog B is known under a number of different names such as Disco-Interacting Protein 2 Homolog B; DIP2 Disco-Interacting Protein 2 Homolog B (Drosophila); DIP2 Disco-Interacting Protein 2 Homolog B; DIP2 Homolog B; and KIAA1463. External Ids for DIP2B Gene are HGNC: 29284; Entrez Gene: 57609; Ensembl: ENSG00000066084; OMIM: 611379; and UniProtKB: Q9P265.

DPYSL2 or Dihydropyrimidinase Like 2 is known under a number of different names such as Dihydropyrimidinase-Related Protein 2 ; Unc-33-Like Phosphoprotein 2; CRMP-2; ULIP-2 ; CRMP2; DRP-2; ULIP2; N2A3 Collapsin

Response Mediator Protein HCRMP-2; Collapsin Response ME,^{,}diator Protein 2; Dihydropyrimidinase-1 jke 2; I)1411t1′2; and D1-⁻W2. External Ids for DPYSL2 Gene arefIGNC: 3014: Entrez Gene: 1808; Enseinbi: ENSG00000092964: OMIM: 602463: and tiniProtKII: Q16555.

FGFR1 or Fibroblast Growth Factor Receptor 1 is known under a number of different names such as Basic Fibroblast Growth Factor Receptor 1; Fms-Related Tyrosine Kinase 2; Proto-Oncogene C-Fgr; EC 2.7.10.1; BFGF-R-1; FGFR-1; BFGFR; FGFBR; FLT-2; HBGFR; FLT2; CEK; FLG; Heparin-Binding Growth Factor Receptor; FMS-Like Tyrosine Kinase 2; Hydroxyaryl-Protein Kinase; Fms-Like Tyrosine Kinase 2; FGFR1/PLAG1 Fusion; Pfeiffer Syndrome; CD331 Antigen; EC 2.7.10; HRTFDS; CD331; N-SAM; N-Sam; ECCL; KAL2; HH2; and OGD. External Ids for FGFR1 Gene are HGNC: 3688; Entrez Gene: 2260; Ensembl: ENSG00000077782; OMIM: 136350; and UniProtKB: P11362.

FOXA1 or Forkhead Box A1 is known under a number of different names such as Hepatocyte Nuclear Factor 3-Alpha; Forkhead Box Protein Al; Transcription Factor 3A; 1-HNF-3-Alpha; HNF-3A; TCF-3A;1-HNF3A; TCF3A; and Hepatocyte Nuclear Factor 3, Alpha. External Ids for FOXA1 Gene are HGNC: 5021; Entrez Gere: 3169; 1:risonibi: ENS000000129514; ° MEM: 602294; and UniProtKII: P55317.

GDF15 or Growth Differentiation Factor 15 is known under a number of different names such as Prostate Differentiation Factor; Non-Steroidal Anti-Inflammatory Drug-Activated Gene-1; Placental Bone Morpliogenetic Protein; Growth/Differentiation Factor 15; Macrophap tnhibitory Cytokine 1; NSA 1D-Activated Gene I Protein; NSAID-Ilegulated Gene 1 Protein; Placental TGF-Beta; GDF-15; MitC-1; NAG-1; NRG-1; PTGFB: PLEB; PDF; NSMD (Nonsteroidal Anti-Inflammatory Drug)-Activated Protein 1; Macrophage Inhibitory Cytokine PTGF-Beta. External Ids for GDF15 Gime are HGNC: 30142; Entrez Gene: 9518; Ensembl: ENSG00000130513; OMI M: 605312; and UniProtKB: Q99988.

HMBOX1 or Hoineobox Containing 1; Homeobox Telomere-Binding Protein 1; Telomere Associated Homeobox-Containing Protein 1; Homeobox-Containing Protein 1 HOT1; Homeobox-Containing Protein PBHNF; HNT1LA; PBHNF; and TAH1. External Ids for HMBOX1 Gene are HGNC: 26137: Entrez Gene: 79618; Ensembl: ENSG00000147421; and UniProtKB: Q6NT76.

KIF13B or Kinesin Family Member 13B is known under a number of different names such as Kine sin-Like Protein KW :13B; Kinesin-Like Protein GAK1N; GA. SIN; Guanyiato Kinase Associated Kinesin; Kinesin 13B; and KIAA0639. External Ids for KIF13B Gene are HGNC: 14405; Entrez Gene: 23303: Ensembl: ENSG00000197892; OMIM: 607350; and UniProtKB: Q9NQT8.

MCPH1 is known under a number of different names such as Microcephalin 1; BRCT-Repeat Inhibitor Of TERT Expression 1; Microcephalin; Microcephaly, Primary Autosomal Recessive 1; Truncated Microcephalin; BRIT1; and MCT. External Ids for MCPH1 Gene are HGNC: 6954; Entrez Gene: 79648; Ensembl: ENSG00000147316; OMIM: 607117; and UniProtKB: Q8NEMO.

MDK or Midkine is known under a number of different names such as Neurite Outgrovyth-Promoting Factor 2; Neurite Growth-Promoting Factor 2; Aniphiregulin-Associated Protein; Midgestation And Kidney Protein; NEGF2; RAP; MK; Neurite Outgrowth-Promoting Protein; Retinoic Acid Inducible Factor; and MK1. External Ids for MDK Gene are HONG; 6972; Entrez Gene: 4192; Ensembl: ENSG00000110492; OMIM: 162096; and UniProtKB: P21741.

MRPL13 or Mitochondrial Ribosomal Protein L13 is known under a number of different names such as Mitochondrial Large Ribosomal Subunit Protein UL13m; 39S Ribosomal Protein L13, Mitocbondrial; MRP-L13; L13mt; RPM L13; RPL13;, L13A; and L13. External Ids for MRPL13 Gene are HGNC: 14278; Entrez Gene: 28998; Ensembk ENSG00000172172; OMIM: 610200; and UniProtKB: Q9HYD1.

NOTCH2 or Notch Receptor 2 is known under a number of different names such as Notch 2; Neurogenic Locus Notch Non Protein 2; HN2; Notch (Drosophita) Floinolog 2; Nothli Homolog 2 (Drosophila); Notch Homolog 2; HJCYS; and AGS2, External Ids for NOTCH2 Gene are 1-HGNC: 7882; Entrez Gene: 4853; Ensembl: ENSG000001.34250; OMIM: 600275; and UniProtK1k Q04721.

PARP8 or Poly(ADP-Ribose) Polymerase Family Member 8 is known under a number of different names such as ADP-Ribosyltransferase Diphtheria Toxin-Like 16; Protein Mono-ADP-Ribosyltransferase PARP8; Poly [ADP-Ribose] Polymerase 8; ARTD16; EC 2.4.2,30; EC 2.4,2,-; PART16; and PARP-8. External Ids for PARP8 Gene are El GNC: 26124; Eritrez Gene: 79668; Ensembl: ENSG-00000151883; and UniProtKB: Q8N3A8.

PDE7A or Phosphodiesterase 7A is known under a number of different names such as High Affinity CAMP-Specific 3′,5′-Cyclic Phosphodiesterase 7A; HCP1; TM22; Phosphodiesterase Isozyme 7; EC 3.1.4.17; EC 3.1.4,53; EC 3.1,4; PDE 7. External Ids for PDE7A Gene are HGNC: 8791; Entrer Gene: 5150; Ensembl: ENS G00000205268; OMIM: 171_885; and UniProtK13: Q13946.

POMK or Protein O-Mannose Kinase is known under a number of different names such as Protein Kinase-Like Protein SgK196; Sugar) Kinase 196; SGK196; Probable Inactive Protein Kinase-Like Protein SgK196; Protein-O-Mannose Kinase; EC 2.7,1.183; NEDDGA12; and MDDG(12. External Ids for POMK Gene are HGNC: 26267; Entrez Gerle: 84197; Ensembl: ENSG00000185900: OMIM: 615247: and UniProtKB: Q9P15K3.

RAB3IL1 is known under a number of different names such as RAB3A interacting Protein Like 1; Guanine Nucleotide Exchange Factor For Rab-3A; RALI3A Interacting Protein 4(tabin3)-Like 1; Rab3A-Interacting-Like Protein 1; Rabin3-Like 1; Guanine Nucleotide Exchange Factor For Rab3A: Rat-3A-Interacting-Like Protein 1; and GRAB. External Ids for RAMILI Gene are HGNC: 9780; Entrez Gene: 5866; Ensembh ENSG00000167994; and UniProtKB: Q8TBN0.

ROCK1 or Rho Associated Coiled-Coil Containing Protein Kinase 1 is known under a number of different names such as Renal Carcinoma Antigen NY-REN-35; Rho-Associated Protein Kinase 1; P160 ROCK-1: EC 2.7.11.I; 60:ROCK; ROCK-I; Rho-Associated, Coiled-Coil-Containing Protein Kinase 1; Rho-Associated, Coiled-Coil-Containing Protein Kinase I; EC 2.7.11.1; and EC 2.7.11. External Ids for ROCK1 Gene are HGNC; 10251; Entrez Gene: 6093; Ensembl: ENSG00000067900; OM M: 601702; and Urn ProtKB: Q13464.

SETD4 or SET Domain Containing 4 is known under a number of different names such as SET Domain-Containing Protein 4; C21orf18: C21orf27; Chromosome 21 Open Reading Frame 27; Chromosome 21 Open Reading Frame 18; and EC 2.1,1.- ,External Ids for SETD4 Gene are HGNC: 1258; Entrez Gene; 54093; Ensembl: ENSG00000185917; and UniProtKB: Q9NVD3.

SLC4A4 is known under a number of different names such as Solute Carrier Family 4 Member 4; Solute Carrier Family 4 (Sodium Bicarbonate Cotransporter),

Member 4; Electrogenic Sodium Bicarbonate Cotransporter 1; Na(+)/LIC03(−) Cotransporter; KNBC1; NBC1: Sodium Bicarbonate Cotransporter 1 (Sodium Bicarbonate Cotransporter, Kidney; Sodium Bicarbonate Cotransporter, Pancreas); Solute Carrier Family 4, Sodium Bicarbonate Cotransporter, Member 4, Brain Type; Solute Carrier Family 4, Sodium Bicarbonate Cotransporter, Member 5; Solute Carrier Family 4, Sodium Bicarbonate Cotransporter, Member 4; Sodium Bicarbonate Cotransporter; NBCel-A; SLC4A5; HNBC1; HhNMC; NBCE1; KNBC; NB ;2: PNBC; NBC. External Ids for SLC4A4 Gene are HGNC: 11030; Entree Gene: 8671; Enscuubl: ENSG00000080493; OMIM; 603345; and Urn ProtKB: Q9Y6ftl.

SMS D4 is known under a number of different names such as SMAD Family Member 4; Deletion Target In Pancreatic Carcinoma 4; Mothers Against Decapentaplegic Homolog 4; MAD Homolog 4; MADH4; DPC4; MAD, Mothers Against. Decapentaplegic Homolog 4 (Drosophila); Mothers Against Decapentaptegic, Drosophila, Homolog Of, 4; SMAD, Mothers Against :DPP Homolog 4 (Drosophila); Deleted In Pancreatic Carcinoma Locus 4; SMAD, Mothers Against DPP Homolog 4; Mothers Against DPP Homolog 4;SMAD HSMAD4; MYHRS; Smad4; and Jilt'. External Ids for SMAD4 Gene are HGNC: 6770; Entrez Gene: 4089; Ensembl: ENSG00000141646; OMIM 600993; and UniProtKB: Q13485.

THAP7 is also known as THAP Domain Containing 7 or THAI Domain-Containing Protein 7. External Ids for THAP7 Gene are HGNC: 23190; Entrez Gene: 80764; Ensembl: ENSG00000184436; OMIM: 609518; and UniProtKB: Q9BT49.

THBS1 or Thrombospondin 1 is known under a number of different names such as Thrombospondin-1p180; Thrombospondi n-1; Glycoprotein G; TSP1; TSP; Thrombosnondin-P50; THBS-1; TSP-1; THBS. External Ids for THBSI Gene are HGNC: 11785; Entrez Gene: 7057: Ensembl: ENSG00000137801; OMIM: 188060; and UniProtEB: P07996.

TNC or Tenascin C is known under a number of different names such as Glioma-Associated-Extracellular Matrix Antigen: Deafness, Autosomal Dominant 56; Hexabrachion (Tenascin); Myotendinous Antigen; Neuronectin; Cytotactin; GP 150-225: Tenascin; GMEM; TN-C; HXB; JI; TN; Hexabrachion (Tenascin C. Cytotactin); Tenascin-C Additional Domain 1; Tenascin-C koform 14/AD1/16; Hexabrachion; Tenascin-C; 150-225; DFNA56; and GP, External Ids for TNC Gene are HGNC: 5318; IIIiitrez Gene: 3371; Ensemble: ENSG00000041982; OMIM; 187380; and UniProtKB: P24821.

TNKS or Tankyrase is known under a number of different names such as Tankyrase, TRF1-Interacting Ankyrin-Related ADP-Ribose Polymerase; TRF1-Interacting Ankyrin-Related_ADP-Ribose Polymerase 1; Protein Poly-A DP-Ribosyltransferase kyrase-1; Ai P-Ribosyltransferase Diphtheria Toxin-Like 5; Poly [ADP-Ribose] Polymerase Tankyrase-1; Poly [ADP-Ribose] Polymerase 5A; Tankyrase Tankyrase-1; PARP5A: TNKS-1; ARTD5; PARPL; TANK1: TINF1: TNKS1; TIN1; TRF1-Interaeting Ankyrin-Related ADP-pilose Polymerase; EC 2.4.2,30; EC 2.4,2.- ; PAP-5a: and PARTS. External Ids for TNKS Gene are HGNC: 11941; Entrez Gene: 8658; Ensernbl: ENSCI-00000173273; OMIM: 603303; and UniProtKB: O95271.

TSHZ2 or Teashirt Zinc Finger Homeobox 2 is known under a number of different names such as Zinc Finger Protein 218; Ovarian Cancer-Related Protein 10-2; Teashirt Family Zinc Finger 2; Teashirt. Homolog 2; C20orf17; OVC10-2; ZNIF218; TSH2; Serologically Defined Colon Cancer Antigen 33 Like; Chromosome 20 Open Reading Frame 17; Cell Growth-Ininbiting Protein 7; and ZABC2. External Ids for TSHZ2 Gene are fiGNc: mow; Frarez Gene; 128553; Ensembl: ENSG-00000182463; OMIM: 614118: and UniProtK13; Q9NRE2.

VTCN1 or V-Set Domain Containing T Cell Activation Inhibitor 1 is known under a number of different names such as V-Set Domain-Containing T-Cell Activation Inhibitor 1; Immime Costimulatory Protein 117-114; B7 Superfamily Member 1; B7 Family Member, H4; B7 Homolog 4; B7-H4; B7h,5; B7H4; T Cell Costinrulatory Molecule 117x; T-Cell Costimulatory Molecule B7x; Protein B7S1; PRO1291; VCTN1: and B7X. External Ids for VTCN1 Gene are HGNC: 28873; Entrez Gene: 79679; Ensemhl: ENSG00000134258: OMIM: 608162; and Uni ProtKB: Q7Z7D3.

WHSC1L1 or NSD3 is known under a number of different names such as Nuclear Receptor Binding SET Domain Protein 3; WHSCI-Like 1 Isoform 9 With Methyltransferase Activity To Lysine; Wolf-Hirschhorn Syndrome Candidate 1-Like 1; Histone-Lysine N-Tvlethyltransf(⁻irase NSD3; Nuclear SET Domain-Containing Protein 3; Protein Whistle; EC 2.1.1.43; WHSC11,1; Wotf-Hirsehhorn Syndrome Candidate 1-Like Protein 1; WHSC1 -Like Protein 1; WHISTLE; Pp14328; KM:T3F: and KMT3G. External his for NS1t3 Gene are HGNC: 12767; Entrez Gene: 54904; Elise⁻a⁻fhb ENSG00000147548; OMIM: 607083: and UniProt Q911Z95.

ZMYM2 is known under a number of different names such as Zinc Finger MYM-Type Containing 2; Zinc :Finger Protein 198; Fused In Myeloproliferative Disorders Protein; Zinc :P′inger MYM-Type Protein 2; ZNF198; RAMP; FIM; Rearranged In Atypical Myeloproliferative Disorder Protein: Rearranged in An Atypical Myeloproliferative Disorder; Zinc Finger, MYM-Type 2; SCLL; and MYM. External Ids for ZMYM2 Gene are HGNC: 12989: Entrez Gene: 7750: Ensembl: ENSG00000121741; OMIM: 602221; and UniProtKB: Q9UBW7:

PMEPA1 is known under a number of different names such as Prostate Transineinbrane Protein, Androgen Induced 1; Transmembrane, Prostate Androgen Induced RNA; Solid Tumor-Associated 1 Protein; Protein TMEPA1; TMEPAI; STAG1: Prostate Transmembrane Protein Androgen Induced 1; Transmembrane Prostate Androgen-Induced Protein; and Solid Tumor-Associated 1. External Ids for PM EPA1 Gene are HGNC: 14107; Entrez Gene: 56937; Ensebl: ENSG00000124225; OMIM: 606564: and UniProtR1:1: Q969W9.

RAD 51 is known under a number of different names such as RAD51 Recombinase; BRCA1/BRCA2-Containing Complex, Subunit 5: DNA Repair Protein RAD51 Homolog 1; RAD51 Homolog A; HRAD51; RAD51A: RECA; RAD51 Hoinotog (RecA Homolog, E. Coli) (S. Cerevisiae); RAD51 (S. Cerevisiae) Homolog (E Coli RecA Homolog); RAD51 Homolog (S. Ceievisiae): RecA, E. Coli, Homolog Of; Recombination Protein A; RecA-Like Protein; HsT16930; HsRad51; HsRAD51; BRCC5; FANCR; and MRMV2. External Ids for RAD51 Gene are HGNC: 9817: Entrez Gene: 5888; Ensembl: ENSG00000051180: OMIM: 179617: amd UniProtKB: Q06609.

STMN2 is known under a number of different names such as Stallunin 2; Superior Cervical Ganglion-10 Protein; Stathmin-Like 2; Stallunin-2; SCGN10; SCG10; Neuronal Growth -Associated Protein (Silencer Element); Superior Cervical Ganglia, Neural Specific 10; Neuron-Specific Growth-Associated Protei; and Protein SCG10; External Ids for STMN2 Gene are HGNC 10577; Entrez Gene: 11075; Ensembl: ENSG0000104435; OMIM: 600621; UniProtKB: Q93045,

Various NRG1 fusion genes are described in. Dhanasekaran et al. Nature Communications 5:5893, 2014, and in Jonna et al., Clin Cancer Res Aug. 15 2019, 25 (16) 4966-4972.

The invention further provides a bispecific antibody that comprises a first antigen-binding site that binds an extracellular part of ErbB-2 and a second antigen-binding site that binds an extracellular part of ErbB-3 for use in the treatment of a subject that is at risk of having an ErbB-2 and ErbB-3 positive cancer, cells of which cancer comprise an NRG1 fusion gene comprising at least a portion of the NRG1-gene fused to a sequence from a different chromosomal location, and which subject is a pretreatment cancer subject that had received a previous ErbB-1; ErbB-2 or ErbB-3 targeted tumor treatment.

Further provided is a method of treating a subject that is at risk of having an ErbB-2 and ErbB-3 positive cancer, cells of which cancer comprise an NRG1 fusion gene comprising at least a portion of the NRG1-gene fused to a sequence from a different chromosomal location, and which subject is a pretreatment cancer subject that had received a previous treatment of chemotherapy or ErbB-2 or ErbB-3 targeted tumor treatment, the method comprising administering to the subject in need thereof a bispecific antibody that comprises a first antigen-binding site that binds an extracellular part of ErbB-2 and a second antigen-binding site that binds an extracellular part of ErbB-3.

A subject is at risk of having the mentioned cancer when the subject is in remission of cancer as a result of a treatment with chemotherapy, an ErbB-1; ErbB-2 or ErbB-3 targeted tumor treatment. Such subjects may be prophylactically treated with a bispecific antibody according to the invention.

The chemotherapy according to the present invention preferably comprises gemcitabine, capecitabine, carboplatin, a taxane, such as docetaxel or paclitaxel, 5-fluorouracil (with or without radiotherapy), vinorelbine, mitoxantrone, vinblastine, cisplatin (or pemetrexed), oxaliplatin, carboplatin, ifosfamide, mytomycine C, vindesine, etoposide, Folfox (i.e. a combination of 5-fluorouracil, leucovorin, and oxaliplatin) or Folfiri (i.e. a combination of leucovorin, 5-fluorouracil and irinotecan),Folfirinox (a combination of leucovorin, 5-fluorouracil, irinotecan and oxaliplatin) or any combination thereof.

In a preferred embodiment, the chemotherapy comprises gemcitabine, capecitabine, 5-fluorouracil (alone or in combination with radiotherapy), Folfirinox, gemcitabine in combination with albumin-bound paclitaxel, gemcitabine in combination with erlotinib for subjects diagnoses with a pancreatic cancer, preferably pancreatic ductal adenocarcinoma (PDAC).

In a preferred embodiment, the chemotherapy comprises gemcitabine in combination with a platinum analogue (such as cisplatin, oxaliplatin and/or, carboplatin), a taxane (such as paclitaxel or docetaxel) in combination with a platinum analogue (such as cisplatin, oxaliplatin and/or, carboplatin), pemetrexed in combination with a platinum analogue (such as cisplatin, oxaliplatin and/or, carboplatin). ifosfamide, mytomycine C, vindesine, vinblastine, etoposide and/or vinorelbine for subjects diagnoses with a lung cancer, preferably non-small cell lung cancer (NSCLC). Said chemotherapy for subjects diagnoses with a lung cancer, preferably non-small cell lung cancer (NSCLC) can be in combination with an anti-EGFR agent (such as erlotinib or cetuximab), anti-vascular endothelial growth factor therapy, bevacizumab and/or a VEGF-trap, such as ziv-aflibercept.

Preferably, the chemotherapy comprises or consists of a combination of gemcitabine and capecitabine and/or Folfiri in the treatment of a pancreas cancer, such as pancreas adenocarcinoma or a liver cancer, including liver metastasis. Cells of which cancer preferably comprise an NRG1 gene fused to ATP1B1.

Administration of the bispecific antibody of the invention comprises a bi-weekly dose of 750 mg antibody.

Preferably, the chemotherapy comprises or consists of a combination of carboplatin and pemextred, preferably administered in combination with pembrolizumab, in the treatment of a lung cancer, preferably lung adenocarcinoma. Cells of which cancer preferably comprise an NRG1 gene fused to CD74. Administration of the bispecific antibody of the invention comprises a bi-weekly dose of 750 mg antibody.

Preferably, the chemotherapy comprises or consists of a combination of gemcitabine and capecitabine in the treatment of pancreas adenocarcinoma. The combination of gemcitabine and capecitabine is preferably followed by Folfiri administration before the bispecific antibody of the present invention is administered. Cells of which cancer preferably comprise an NRG1 gene fused to CD74. Administration of the bispecific antibody of the invention comprises a bi-weekly dose of 750 mg antibody.

Antigen-binding sites in an antibody are typically present in the variable domains. The variable domains comprise a heavy chain variable region and a light chain variable region.

The subject has preferably undergone chemotherapy or a therapy that targeted towards ErbB-2 inhibition. Inhibition of ErbB-2 signaling also referred to as ErbB-2 targeted tumor treatment preferably comprises administration of a monospecific bivalent antibody comprising antigen-binding sites that bind an extracellular part of ErbB-2; or administration of a ErbB-2 tyrosine kinase inhibitor (TKI) or a combination thereof, and wherein the monospecific bivalent antibody is preferably trastuzumab, pertuzumab, or trastuzumab-emtansine and wherein the ErbB-2 TKI is preferably one or more of lapatinib, canertinib, neratinib, tucatinib (irbinitinib), CP-724714, tarloxitinib, mubritinib, afatinib, varlitinib, and dacomitinib, preferably lapatinib, canertinib, neratinib, tucatinib (irbinitinib), mubritinib, afatinib, varlitinib, or dacomitinib, more preferably afatinib.

A method of treatment or bispecific antibody for use in the treatment as described in the present invention preferably further determining whether the cell comprises an NRG1-fusion or whether the tumor comprises cells with an NRG1-fusion. This can for instance be done on cells of a biopsy. Various methods are available and many are known in the art. Known that region wherein the chromosome break occurs in the case of NRG1-fusions it is routine for the skilled person to determine whether a tumor comprises such an NRG1-fusion. One way is by means of PCR-amplification with primers that span the junction in the NRG1 fusion. This can easily be implemented for NRG1-fusions that are known to occur. New fusions can also be detected easily. A suitable way is for instance by junction cloning techniques used to find for instance the integration site of retroviral genomes. A suitable method is by means of LAM-PCR see Schmidt et al Nature Methods 4, 1051-1057 (2007) doi:10.1038/nmeth1103 and specific references to the LAM-technology therein.

Techniques available to identify putative NRG1-fusions include RNA-based methodology, including Anchored multiplex PCR, nCounter, RT-PCR,

Transcriptome analyses, FISH, DNA-based methodologies including Hybrid capture-based next generation sequencing (NGS), Amplicon-based NGS, among other techniques available commercially.

Various methods are available to determine the level of ErbB receptors on a cell of a cancer. Examples are immunohistochemistry or fluorescence in situ hybridization. The HercepTestTM and/or HER2 FISH (pharm Dx™), marketed both by Dako Denmark A/S, and/or using a HERmark® assay, marketed by Monogram Biosciences are examples of suitable assays for determining ErbB-2 or ErbB-3 cell surface receptor density. Other methods for determining the ErbB-2 receptor cell density are well-known to a skilled person. In vivo methods for determining ErbB-2 are also known, see, e.g., Chernomoridik et al. Mol Imaging. 2010 Aug; 9(4): 192-200 and Ardeshirpour et al. Technol Cancer Res Treat. 2014 Oct; 13(5): 427-434. Preferably, the methods disclosed herein further comprise determining the ErbB-2 cell-surface receptor density for said cell or tumor. Such methods are known to a skilled person (see, e.g., van der Woning and van Zoelen Biochem Biophys Res Commun. 2009 Jan. 9; 378(2):285-9). Preferably, the methods disclosed herein further comprise determining the ErbB-1 cell-surface receptor density for said cell or tumor. Such methods are known to a skilled person (see, e.g., EGFR pharmDx™Kit (Dako)) amd McDonagh et al. Mol Cancer Ther 2012; 11:582). Similar methods can be used to determine ErbB-4 cell-surface receptor density. In some embodiments, the ErbB-1, ErbB-2, ErbB-3, and ErbB-4 cell-surface receptor densities are determined by FACS analysis on biopsied tumor cells.

The amount of bispecific antibody to be administered to a subject is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. The selected dosage level will depend upon a variety of factors including the route of administration, the time of administration, the rate of excretion of the particular compound being employed, the duration of the treatment, other drugs, compounds and/or materials used in combination, the age, sex, weight, condition, general health and prior medical history of the subject being treated, and like factors well known in the medical arts. The dosage can be in the range of the dosing regimen for trastuzumab or lower.

With respect to the bispecific antibody of the invention, it has a good safety profile at relatively high doses, thus providing a large therapeutic window, and making it a good partner for combination therapy. Dosing of the bispecific antibody of the present invention follows a weekly, biweekly or tri-weekly administration regimen of 750 mg, preferably a bi-weekly or tri-weekly dose of 750 mg. The dosing is preferably in subjects with pancreatic cancer, NSCLC or a solid tumor, and includes any subject having a solid tumor harboring an NRG1-fusion, wherein such subject has progressed upon the administration of chemotherapy or an ErbB-2 or ErbB-3 targeting agent or TKI. Alternatively, a dosing regimen is followed comprising a weekly flat dose administration of 400 mg, preferably commenced after a single administration of 800 mg. Following this alternative dosing regimen, the bispecific antibody of the invention is preferably administered in a weekly dose of 400 mg for 3 weeks followed by 1 week without dosing. Next, one or more cycles of a period of four weeks, consisting of three weekly flat dosages of 400 mg, followed by a week without administration is followed. This is preferably followed until a therapeutic effect is observed.

Dosing preferably involves intravenous injections of two infusions of the bispecific antibody of the invention to arrive at the complete dose, preferably when dosing >360 mg antibody. Alternatively, a single infusion of the complete dose may be given for lower dosages, for instance when dosing <360 mg antibody. Pre-medication maybe included in the dosing regimen to mitigate infusion-related reactions.

In a preferred embodiment, treatment includes administering of a bispecific antibody to a subject having NSCLC, preferably metastatic disease in lung and pleura, and cancer cells with a SDC4-NRG1 fusion that has a first antigen-binding site that binds an extracellular part of ErbB-2 and a second antigen-binding site that binds an extracellular part of ErbB-3 of a patient that received afatinib prior-treatment. Preferably, treatment comprises stabilization of the tumor in terms of size or lesions or prevention of further tumor growth, including tumor reduction. Preferably, treatment or administration is with the bispecific antibody according to the invention on a weekly regimen and proceeds for a period of at least 1, 2, 4, 8 or at least 12 months. Preferably, a dosing regimen is followed comprising a weekly cycle with a flat dose of 400 mg weekly commenced after an initial administration of 800 mg. From week 3, the bispecific antibody of the invention is given at a weekly dose of 400 mg for 3 weeks followed by 1 week without dosing of the bispecific antibody of the invention. Alternatively, a dosing regimen is followed comprising a bi-weekly cycle with a flat dose of 750 mg weekly commenced after an initial administration of a 750 mg infusion over a four-hour period, followed by a bi-weekly two-hour infusion of 750 mg in a four-week cycle. A further alternative comprises a tri-weekly administration of a flat dose of 750 mg per subject.

Preferably, diagnosis involves molecular profiling using a targeted sequencing method, analysis of at last one biomarker, including fusions, insertion/deletions (indels), single nucleotide variants and/or copy number variations. Preferably, the tumor genome shows absence of mutations in one or more genes selected from the group consisting of EGFR, KRAS, cKIT-BRCA1-2, MET, ROS, RET, ALK.

Preferably, disease progression comprises a measuring of anti-tumor activity in the lung by using a CT-scan and assessment by RECIST v1.1 determining objective overall response rate (ORR), duration of response (DOR), progression-free survival (PFS) and survival. Preferably, a bispecific antibody that has a first antigen-binding site that binds an extracellular part of ErbB-2 and a second antigen-binding site that binds an extracellular part of ErbB-3, in particular MF3958×MF3178, stabilizes tumors in terms of size or lesions or the treatment prevents further tumor growth. Preferably, treatment is without drug related toxicity or has a good safety profile with limited occurrence of grade 3-5 adverse events that are actual or suspected to be drug related.

The bispecific antibodies can be formulated as a pharmaceutical composition comprising a pharmaceutically acceptable carrier, diluent, or excipient, and additional, optional, active agents. The antibodies and compositions comprising the antibodies can be administered by any route including parenteral, enteral, and topical administration. Parenteral administration is usually by injection, and includes, e.g., intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, sub arachnoid, intraspinal, intracerebro spinal, intratumoral, and intrasternal injection and infusion.

The disclosure provides bispecific antibodies for use in the methods and treatments described herein. Suitable bispecific antibodies comprise a first antigen-binding site that binds ErbB-2 and a second antigen-binding site that binds ErbB-3. The bispecific antibody reduces or can reduce a ligand-induced receptor function of ErbB-3 on an ErbB-2 and ErbB-3 positive cell and/or disrupt ErbB-2 and ErbB-3 heterodimerization. Preferred antibodies and their preparation are disclosed in WO 2015/130173, which is hereby incorporated by reference. The examples in WO 2015/130173 further describe a number of properties of the antibodies, such as ligand binding and epitope mapping.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on or administering an active agent or combination of active agents to a subject with the objective of curing or improving a disease or symptom thereof. This includes reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, a therapeutic amount is an amount sufficient to delay tumor development. In some embodiments, a therapeutic amount is an amount sufficient to prevent or delay tumor recurrence.

The therapeutic amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

A therapeutic amount may vary according to factors such as the disease state, age, sex, and weight of the individual to be treated, and the ability of the agent or combination of agents to elicit a desired response in the individual. A therapeutic amount can be administered in one or more administrations. A therapeutic amount also includes an amount that balances any toxic or detrimental effects of the agent or combination of agents and the therapeutically beneficial effects.”

As used herein, the term “antigen-binding site” refers to a site derived from and preferably as present on a bispecific antibody which is capable of binding to antigen. An antigen-binding site is typically formed by and present in the variable domain of the antibody. The variable domain contains said antigen-binding site. A variable domain that binds an antigen is a variable domain comprising an antigen-binding site that binds the antigen.

In one embodiment an antibody variable domain comprises a heavy chain variable region (VH) and a light chain variable region (VL). The antigen-binding site can be present in the combined VH/VL variable domain, or in only the VH region or only the VL region. When the antigen-binding site is present in only one of the two regions of the variable domain, the counterpart variable region can contribute to the folding and/or stability of the binding variable region, but does not significantly contribute to the binding of the antigen itself.

As used herein, antigen-binding refers to the typical binding capacity of an antibody to its antigen. An antibody comprising an antigen-binding site that binds to ErbB-2, binds to ErbB-2 and, under otherwise identical conditions, at least 100-fold lower to the homologous receptors ErbB-1 and ErbB-4 of the same species. An antibody comprising an antigen-binding site that binds to ErbB-3, binds to ErbB-3 and, under otherwise identical conditions, not to the homologous receptors ErbB-1 and ErbB-4 of the same species.

Considering that the ErbB-family is a family of cell surface receptors, the binding is typically assessed on cells that express the receptor(s). Binding of an antibody to an antigen can be assessed in various ways. One way is to incubate the antibody with the antigen (preferably cells expressing the antigen), removing unbound antibody (preferably by a wash step) and detecting bound antibody by means of a labeled antibody that binds to the bound antibody.

Antigen binding by an antibody is typically mediated through the complementarity regions of the antibody and the specific three-dimensional structure of both the antigen and the variable domain allowing these two structures to bind together with precision (an interaction similar to a lock and key), as opposed to random, non-specific sticking of antibodies. As an antibody typically recognizes an epitope of an antigen, and as such epitope may be present in other compounds as well, antibodies according to the present invention that bind ErbB-2 and/or ErbB-3 may recognize other proteins as well, if such other compounds contain the same epitope. Hence, the term “binding” does not exclude binding of the antibodies to another protein or protein(s) that contain the same epitope. Such other protein(s) is preferably not a human protein. An ErbB-2 antigen-binding site and an ErbB-3 antigen-binding site as defined herein typically do not bind to other proteins on the membrane of cells in a post-natal, preferably adult human. A bispecific antibody as disclosed herein is typically capable of binding ErbB-2 and ErbB-3 with a binding affinity of at least lx10e-6 M.

The term “interferes with binding” as used herein means that the antibody is directed to an epitope on ErbB-3 and the antibody competes with ligand for binding to ErbB-3. The antibody may diminish ligand binding, displace ligand when this is already bound to ErbB-3 or it may, for instance through steric hindrance, at least partially prevent that ligand can bind to ErbB-3.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity The specificity determines which antigen or epitope thereof is specifically bound by the binding domain. The affinity is a measure for the strength of binding to a particular antigen or epitope. Typically, antibodies for therapeutic applications have affinities of up to1×10e-10 M or higher. Antibodies such the bispecific antibodies of the present invention comprise the constant domains (Fc part) of a natural antibody. An antibody of the invention is typically a bispecific full length antibody, preferably of the human IgG subclass. Preferably, an antibody as disclosed herein is of the human IgG1 subclass. Such antibodies have good ADCC properties, have favorable half-life upon in vivo administration to humans and CH3 engineering technology exists that can provide for modified heavy chains that preferentially form heterodimers over homodimers upon co-expression in clonal cells.

An antibody as disclosed herein is preferably a “full length” antibody. The term ‘full length’ is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL (suitable amino acid sequences for the respective domains are depicted in FIGS. 1 and 2). An antibody binds to antigen via the variable domains contained in the Fab portion, and after binding can interact with molecules and cells of the immune system through the constant domains, mostly through the Fc portion. The terms ‘variable domain’, ‘VH/VL pair’, ‘VH/VL’ are used herein interchangeably. Full length antibodies according to the invention encompass antibodies wherein mutations may be present that provide desired characteristics. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region. For instance, ADCC activity of an antibody can be improved when the antibody itself has a low ADCC activity, by slightly modifying the constant region of the antibody (Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489).

Full length IgG antibodies are preferred because of their favorable half-life and the need to stay as close to fully autologous (human) molecules for reasons of immunogenicity. An antibody as disclosed herein is preferably a bispecific IgG antibody, preferably a bispecific full length IgG1 antibody. IgG1 is favored based on its long circulatory half-life in man. In order to prevent any immunogenicity in humans it is preferred that the bispecific IgG antibody is a human IgG1.

The term ‘bispecific’ (bs) means that one part of the antibody (as defined above) binds to one epitope on an antigen whereas a second part binds to a different epitope. The different epitope is typically present on a different antigen. The heavy chain variable regions of the bispecific antibody are typically different from each other, whereas the light chain variable regions are preferably the same. A bispecific antibody wherein the different heavy chain variable regions are associated with the same, or a common, light chain is also referred to as a bispecific antibody with a common light chain. A bispecific antibody as described herein typically comprises one variable domain that binds ErbB-2 and another variable domain that binds ErbB-3.

Preferred bispecific antibodies can be obtained by co-expression of two different heavy chains and a common light chain in a single cell. When wildtype CH3 domains are used, co-expression of two different heavy chains and a common light chain will result in three different species, AA, AB and BB. To increase the percentage of the desired bispecific product (AB) CH3 engineering can be employed, or in other words, one can use heavy chains with compatible heterodimerization domains, as defined hereunder. Suitable compatible CH3 heterodimerization domains are depicted in FIGS. 2d and 2e.

The term ‘compatible heterodimerization domains’ as used herein refers to protein domains that are engineered such that engineered domain A′ will preferentially form heterodimers with engineered domain B′ and vice versa, whereas homodimerization between A′-A′ and B′-B′ is diminished.

The term ‘common light chain’ refers to light chains which may be identical or have some amino acid sequence differences while the binding specificity of the full length antibody is not affected . It is for instance possible, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common VL’, ‘single light chain’, ‘single VL’, with or without the addition of the term ‘rearranged’ are all used herein interchangeably.

A common light chain (variable region) preferably has a germline sequence. A preferred germline sequence is a light chain variable region that is frequently used in the human repertoire and has good thermodynamic stability, yield and solubility. In a preferred embodiment the light chain comprises a light chain region comprising the amino acid sequence of an IgVκ1-39*01 gene segment as depicted in the Sequences 1C “Common light chain IGKV1-39/jk1” with 0-10, preferably 0-5 amino acid insertions, deletions, substitutions, additions or a combination thereof. IgW1-39 is short for Immunoglobulin Variable Kappa 1-39 Gene. The gene is also known as Immunoglobulin Kappa Variable 1-39; IGKV139; or IGKV1-39. External Ids for the gene are HGNC: 5740; Entrez Gene: 28930; Ensembl: ENSG00000242371. The variable region of IGKV1-39 is listed in the FIG. 1. The V-region can be combined with one of five J-regions. FIG. 1 describe two preferred sequences for IgW1-39 in combination with a J-region. The joined sequences are indicated as IGKV1-39/jkl and IGKV1-39/jk5; alternative names are IgVκ1-39*01/IGJκ1*01 or IgVκ1-39*01/IGJκ5*01 (nomenclature according to the IMGT database worldwide web at imgt.org).

It is preferred that the IgVκ1-39*01 comprising light chain variable region is a germline sequence. It is further preferred that the IGJκ1*01 or /IGJκ5*01 comprising light chain variable region is a germline sequence. In a preferred embodiment, the IGκV1-39/jk1 or IGKV1-39/jk5 light chain variable regions are germline sequences.

In a preferred embodiment the light chain variable region comprises a germline IgVx1-39*01. In a preferred embodiment the light chain variable region comprises the kappa light chain IgVx1-39*01/IGJK1*01 or IgVx1-39*01/IGJK5*01. In a preferred embodiment a IgVx1-39*01/IGJK1*01. The light chain variable region preferably comprises a germline kappa light chain IgVx1-39*01/IGJK1*01 or germline kappa light chain IgVx1-39*01/IGJK5*01, preferably a germline IgVK1-39*01/IGJK1*01.

Obviously, those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions. The light chain can also be a light chain as specified herein above, having 1-5 amino acid insertions, deletions, substitutions or a combination thereof.

Preferably, both the first antigen binding site and said second antigen binding site comprise a light chain variable region comprising a CDR1 having the sequence (RASQSISSYLN), a CDR2 having the sequence (AASSLQS), and a CDR3 having the sequence (QQSYSTPPT).

Antibodies disclosed herein can reduce a ligand-induced receptor function of

ErbB-3 on an ErbB-2 and ErbB-3 positive cell. In the presence of excess ErbB-2, ErbB-2/ErbB-3 heterodimers may provide a growth signal to the expressing cell in the absence of detectable ligand for the ErbB-3 chain in the heterodimer. This

ErbB-3 receptor function is herein referred as a ligand-independent receptor function of ErbB-3. The ErbB-2/ErbB-3 heterodimer also provide a growth signal to the expressing cell in the presence of an ErbB-3 ligand. This ErbB-3 receptor function is herein referred to as a ligand-induced receptor function of ErbB-3. The term “ErbB-3 ligand” as used herein refers to polypeptides which bind and activate ErbB-3. Examples of ErbB-3 ligands include, but are not limited to neuregulin 1 (NRG) and neuregulin 2, betacellulin, heparin-binding epidermal growth factor, and epiregulin. The term includes biologically active fragments and/or variants of a naturally occurring polypeptide. Preferably, the ligand-induced receptor function of ErbB-3 is ErbB-3 ligand-induced growth of an ErbB-2 and ErbB-3 positive cell. In a preferred embodiment said cell is an MCF-7 cell (ATCC® HTB-22TM); an SKBR3 (ATCC® HTB-30TH) cell; an NCI-87 (ATCC® CRL-5822TM) cell; a BxPC-3-1uc2 cell (Perkin Elmer 125058), a BT-474 cell (ATCC® HTB-20TH) or a JIMT 1 cell (DSMZ no.: ACC 589).

As used herein the ligand-induced receptor function is reduced by at least 20%, preferably at least 30, 40, 50 60, or at least 70% in a particularly preferred embodiment the ligand-induced receptor function is reduced by 80, more preferably by 90%. The reduction is preferably determined by determining a ligand-induced receptor function in the presence of a bispecific antibody disclosed herein, and comparing it with the same function in the absence of the antibody, under otherwise identical conditions. The conditions comprise at least the presence of an ErbB-3 ligand. The amount of ligand present is preferably an amount that induces half of the maximum growth of an ErbB-2 and ErbB-3 positive cell line. The ErbB-2 and ErbB-3 positive cell line for this test is preferably the MCF-7 cell line

(ATCC® HTB-22TM), the SKBR3 cell line (ATCC® HTB-30TH) cells, the JIMT 1 cell line (DSMZ ACC 589) or the NCI-87 cell line (ATCC® CRL-5822TM). The test and/or the ligand for determining ErbB-3 ligand-induced receptor function is preferably a test for ErbB-3 ligand induced growth reduction as specified in the examples.

The ErbB-2 protein contains several domains (see for reference FIG. 1 of Landgraf, R Breast Cancer Res. 2007; 9(1): 202-). The extracellular domains are referred to as domains I-IV. The place of binding to the respective domains of antigen-binding sites of antibodies described herein has been mapped. A bispecific antibody with an antigen-binding site (first antigen-binding site) that binds domain I or domain IV of ErbB-2 (first antigen-binding site) comprises a heavy chain variable region that maintains significant binding specificity and affinity for ErbB-2 when combined with various light chains. Bispecific antibodies with an antigen-binding site (first antigen-binding site) that binds domain I or domain IV of ErbB-2 (first antigen-binding site) and an antigen-binding site for ErbB-3 (second antigen-binding site) are more effective in reducing a ligand-induced receptor function of ErbB-3 when compared to a bispecific antibody comprising an antigen-binding site (first antigen-binding site) that binds to another extra-cellular domain of ErbB-2. A bispecific antibody comprising an antigen-binding site (first antigen-binding site) that binds ErbB-2, wherein said antigen-binding site binds to domain I or domain IV of ErbB-2 is preferred. Preferably said antigen-binding site binds to domain IV of ErbB-2. Preferred antibodies comprises a first antigen-binding site that binds domain I of ErbB-2 and a second antigen-binding site that binds domain III of ErbB-3.

In one preferred embodiment, said antibody comprises an antigen-binding site that binds at least one amino acid of domain I of ErbB-2 selected from the group consisting of T144, T164, R166, P172, G179, S180 and R181, and surface-exposed amino acid residues that are located within about 5 amino acid positions from T144, T164, R166, P172, G179, S180 or R181.

In one preferred embodiment, said antibody preferably comprises an antigen-binding site that binds at least one amino acid of domain III of ErbB-3 selected from the group consisting of R426 and surface-exposed amino acid residues that are located within 11.2 Å from R426 in the native ErbB-3 protein.

A bispecific antibody with an antigen-binding site (first antigen-binding site) that binds ErbB-2, and that further comprises ADCC are more effective than other ErbB-2 binding antibodies that did not have significant ADCC activity, particularly in vivo. A bispecific antibody which exhibits ADCC is therefore preferred. It was found that antibodies wherein said first antigen-binding site binds to domain IV of ErbB-2 had intrinsic ADCC activity. A domain I binding ErbB-2 binding antibody that has low intrinsic ADCC activity can be engineered to enhance the ADCC activity Fc regions mediate antibody function by binding to different receptors on immune effector cells such as macrophages, natural killer cells, B-cells and neutrophils. Some of these receptors, such as CD16A (FcγRIIIA) and CD32A (FcγRIIA), activate the cells to build a response against antigens. Other receptors, such as CD32B, inhibit the activation of immune cells. By engineering Fc regions (through introducing amino acid substitutions) that bind to activating receptors with greater selectivity, antibodies can be created that have greater capability to mediate cytotoxic activities desired by an anti-cancer Mab. One technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489). Further provided is therefore a bispecific antibody as disclosed herein, which is afucosylated. Alternatively, or additionally, multiple other strategies can be used to achieve ADCC enhancement, for instance including glycoengineering and mutagenesis, all of which seek to improve Fc binding to low-affinity activating FcyRIIIa, and/or to reduce binding to the low affinity inhibitory FcγRIIb.

Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are typically co-incubated with the antibody-labeled target cells. Target cell lysis is subsequently typically measured by release of intracellular label, for instance by a scintillation counter or spectrophotometry.

In preferred bispecific antibodies, the affinity of said second antigen-binding site for an ErbB 3 positive cell is equal to, or preferably higher than, the affinity of said first antigen-binding site for an ErbB-2 positive cell. The affinity (KD) of said second antigen-binding site for an ErbB-3 positive cell is preferably lower than or equal to 2.0 nM, more preferably lower than or equal to 1.5 nM, more preferably lower than or equal to 1.39 nM, more preferably lower than or equal to 0.99 nM. In one preferred embodiment, the affinity of said second antigen-binding site for ErbB-3 on SK BR 3 cells is lower than or equal to 2.0 nM, more preferably lower than or equal to 1.5 nM, more preferably lower than or equal to 1.39 nM, preferably lower than or equal to 0.99 nM. In one embodiment, said affinity is within the range of 1.39-0.59 nM. In one preferred embodiment, the affinity of said second antigen-binding site for ErbB-3 on BT 474 cells is lower than or equal to 2.0 nM, more preferably lower than or equal to 1.5 nM, more preferably lower than or equal to 1.0 nM, more preferably lower than 0.5 nM, more preferably lower than or equal to 0.31 nM, more preferably lower than or equal to 0.23 nM. In one embodiment, said affinity is within the range of 0.31-0.15 nM. The above-mentioned affinities are preferably as measured using steady state cell affinity measurements, wherein cells are incubated at 4° C. using radioactively labeled antibody, where after cell-bound radioactivity is measured, as described in the Examples of WO 2015/130173.

The affinity (KD) of said first antigen-binding site for an ErbB-2 positive cell is preferably lower than or equal to 5.0 nM, more preferably lower than or equal to 4.5 nM, more preferably lower than or equal to 3.9 nM. In one preferred embodiment, the affinity of said first antigen-binding site for ErbB-2 on SK BR 3 cells is lower than or equal to 5.0 nM, preferably lower than or equal to 4.5 nM, more preferably lower than or equal to 4.0 nM, more preferably lower than or equal to 3.5 nM, more preferably lower than or equal to 3.0 nM, more preferably lower than or equal to 2.3 nM. In one embodiment, said affinity is within the range of 3.0-1.6 nM. In one preferred embodiment, the affinity of said first antigen-binding site for ErbB-2 on BT 474 cells is lower than or equal to 5.0 nM, preferably lower than or equal to 4.5 nM, more preferably lower than or equal to 3.9 nM. In one embodiment, said affinity is within the range of 4.5-3.3 nM. The above-mentioned affinities are preferably as measured using steady state cell affinity measurements, wherein cells are incubated at 4° C. using radioactively labeled antibody, where after cell-bound radioactivity is measured, as described in the Examples of WO 2015/130173.

Preferably, the bispecific antibodies used in the disclosed methods do not significantly affect the survival of cardiomyocytes. Cardiotoxicity is a known risk factor in ErbB 2 targeting therapies and the frequency of complications is increased when trastuzumab is used in conjunction with anthracyclines thereby inducing cardiac stress.

The bispecific antibodies disclosed herein are preferably used in humans. thus, preferred antibodies are human or humanized antibodies. Tolerance of a human to a polypeptide is governed by many different aspects. Immunity, be it T-cell mediated, B-cell mediated or other is one of the variables that are encompassed in tolerance of the human for a polypeptide. The constant region of a bispecific antibody is preferably a human constant region. The constant region may contain one or more, preferably not more than 10, preferably not more than 5 amino-acid differences with the constant region of a naturally occurring human antibody. It is preferred that the constant part is entirely derived from a naturally occurring human antibody. Various antibodies produced herein are derived from a human antibody variable domain library. As such these variable domains are human. The unique CDR regions may be derived from humans, be synthetic or derived from another organism. The variable region is considered a human variable region when it has an amino acid sequence that is identical to an amino acid sequence of the variable region of a naturally occurring human antibody, but for the CDR region. The variable region of an ErbB-2 binding VH, an ErbB-3 binding VH, or a light chain in an antibody may contain one or more, preferably not more than 10, preferably not more than 5 amino-acid differences with the variable region of a naturally occurring human antibody, not counting possible differences in the amino acid sequence of the CDR regions. Such mutations occur also in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; deimmunization of the murine heavy chain variable region, preferably done by removing known or suspected T- or B- cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Such deimmunized murine heavy chain variable regions are less immunogenic in humans than the original murine heavy chain variable region. Preferably a variable region or domain is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the invention is preferably a mammal, more preferably a primate, most preferably a human.

A bispecific antibody disclosed herein preferably comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. Preferably the constant region comprises an IgG constant region, more preferably an IgG1 constant region, preferably a mutated IgG1 constant region. Some variation in the constant region of IgG1 occurs in nature, such as for instance the allotypes Glml, 17 and Glm3, and/or is allowed without changing the immunological properties of the resulting antibody. Typically between about 1-10 amino acid insertions, deletions, substitutions or a combination thereof are allowed in the constant region.

Preferred bispecific antibodies as disclosed herein comprise:

at least the CDR3 sequence, preferably at least the CDR1, CDR2 and CDR3 sequences, or at least the heavy chain variable region sequence, of an ErbB 2 specific heavy chain variable region selected from the group consisting of MF2926, MF2930, MF1849; MF2973, MF3004, MF3958, MF2971, MF3025, MF2916, MF3991, MF3031, MF2889, MF2913, MF1847, MF3001, MF3003 and MF1898, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences; and/or
at least the CDR3 sequence, preferably at least the CDR1, CDR2 and CDR3 sequences, or at least the heavy chain variable region sequence, of an ErbB 3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences.

CDR sequences are for instance varied for optimization purposes, preferably in order to improve binding efficacy or the stability of the antibody. Optimization is for instance performed by mutagenesis procedures where after the stability and/or binding affinity of the resulting antibodies are preferably tested and an improved ErbB 2 or ErbB 3 -specific CDR sequence is preferably selected. A skilled person is well capable of generating antibody variants comprising at least one altered CDR sequence. For instance, conservative amino acid substitution is applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

Preferred antibodies comprise a variable domain that binds ErbB-2, wherein the VH chain of said variable domain comprises the amino acid sequence of VH chain MF2926; MF2930; MF1849; MF2973; MF3004; MF3958 (is humanized MF2971); MF2971; MF3025; MF2916; MF3991 (is humanized MF3004); MF3031; MF2889; MF2913; MF1847; MF3001, MF3003 or MF1898; or comprises the amino acid sequence of VH chain MF2926; MF2930; MF1849; MF2973; MF3004; MF3958 (is humanized MF2971); MF2971; MF3025; MF2916; MF3991 (is humanized

MF3004); MF3031; MF2889; MF2913; MF1847; MF3001, MF3003 or MF1898 as having at most 15, preferably 1, 2, 3, 4, 5, 6, 7, 8, 9 or 10 more preferably at most 1, 2, 3, 4 or 5, amino acid insertions, deletions, substitutions or a combination thereof with respect to the above mentioned VH chain sequence. The VH chain of the variable domain that binds ErbB-2 preferably comprises the amino acid sequence of:

### MF1849; or

MF2971 or a humanized version thereof, wherein said humanized version preferably comprises the amino acid sequence of MF3958; or
MF3004 or a humanized version thereof, wherein said humanized version preferably comprises the amino acid sequence of MF3991. In one embodiment, the VH chain of the variable domain that binds ErbB-2 comprises the amino acid sequence of VH chain MF1849; or MF2971 or a humanized version thereof, wherein said humanized version preferably comprises the amino acid sequence of MF3958; or MF3004 or a humanized version thereof, wherein said humanized version preferably comprises the amino acid sequence of MF3991, wherein the recited VH sequences have at most 15, preferably 1, 2, 3, 4, 5, 6, 7, 8, 9 or 10, more preferably at most 1, 2, 3, 4 or 5, amino acid insertions, deletions, substitutions or a combination thereof with respect to the respective sequence. In a preferred embodiment the VH chain of the variable domain that binds ErbB-2 comprises the amino acid sequence of MF3958; or comprises the amino acid sequence of MF3958 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence.

The VH chain of the variable domain that binds Erb-B3 preferably comprises the amino acid sequence of VH chain MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074; or comprises the amino acid sequence of VH chain MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence. The VH chain of the variable domain that binds Erb-B3 preferably comprises the amino acid sequence of MF3178, MF3176, MF3163, MF6058, MF6061 or MF6065; or comprises the amino acid sequence of MF3178, MF3176, MF3163, MF6058, MF6061 or MF6065 having at most 15, preferably 1, 2, 3, 4, 5, 6, 7, 8, 9 or 10, more preferably in at most 1, 2, 3, 4 or 5, amino acid insertions, deletions, substitutions or a combination thereof with respect to the respective VH chain sequence. In a preferred embodiment the VH chain of the variable domain that binds ErbB-3 comprises the amino acid sequence of MF3178; or comprises the amino acid sequence of MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence. Preferably, the above-mentioned amino acid insertions, deletions and substitutions are not present in the CDR3 region. The above-mentioned amino acid insertions, deletions and substitutions are also preferably not present in the CDR1 and CDR2 regions. The above-mentioned amino acid insertions, deletions and substitutions are also preferably not present in the FR4 region.

Preferably, the antibody comprises at least the CDR1, CDR2 and CDR3 sequences of MF1849, MF2971, MF3958, MF3004 or MF3991, most preferably at least the CDR1, CDR2 and CDR3 sequences of MF3958. Said antibody preferably comprises at least the CDR1, CDR2 and CDR3 sequences of MF3178, MF3176, MF3163, MF6058, MF6061 or MF6065, most preferably at least the CDR1, CDR2 and CDR3 sequence of MF3178.

Preferably, the ErbB-2 specific heavy chain variable region comprises the amino acid sequence of the VH chain MF3958 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH (preferably wherein said insertions, deletions, substitutions are not in CDR1, CDR2, or CDR3). They are also preferably not present in the FR4 region. An amino acid substitution is preferably a conservative amino acid substitution.

Preferably, the ErbB-3 specific heavy chain variable region comprises the amino acid sequence of the VH chain MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH. The one or more amino acid insertions, deletions, substitutions or a combination thereof are preferably not in the CDR1, CDR2 and CDR3 region of the VH chain. They are also preferably not present in the FR4 region. An amino acid substitution is preferably a conservative amino acid substitution.

Preferably, the ErbB-2 specific heavy chain variable region comprises the amino acid sequence of the VH chain MF3991 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH (preferably wherein said insertions, deletions, substitutions are not in CDR1, CDR2, or CDR3). They are also preferably not present in the FR4 region. An amino acid substitution is preferably a conservative amino acid substitution.

Preferably, the ErbB-3 specific heavy chain variable region comprises the amino acid sequence of the VH chain MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH. The one or more amino acid insertions, deletions, substitutions or a combination thereof are preferably not in the CDR1, CDR2 and CDR3 region of the VH chain. They are also preferably not present in the FR4 region. An amino acid substitution is preferably a conservative amino acid substitution.

Preferably, the first antigen-binding site of the antibody comprises at least the CDR1, CDR2 and CDR3 sequences of MF3958, or CDR1, CDR2 and CDR3 sequences that differ in at most three, preferably in at most two, preferably in at most one amino acid from the CDR1, CDR2 and CDR3 sequences of MF3958, and wherein said second antigen-binding site comprises at least the CDR1, CDR2 and CDR3 sequence of MF3178, or CDR1, CDR2 and CDR3 sequences that differ in at most three, preferably in at most two, preferably in at most one amino acid from the CDR1, CDR2 and CDR3 sequences of MF3178.

Preferably, the bispecific antibody comprises i) a first antigen binding site comprising an ErbB-2 specific heavy chain variable region comprising the CDR1, CDR2, and CDR3 sequence of MF3958 and a light chain variable region and ii) a second antigen binding site comprising an ErbB-3 specific heavy chain variable region comprising the CDR1, CDR2, and CDR3 sequence of MF3178 and a light chain variable region.

Preferably, the ErbB-2 specific heavy chain variable region has the MF3958 sequence and the ErbB-3 specific heavy chain variable region has the MF3178 sequence. This combination is also referred to as the PB4188 antibody. Preferably, the PB4188 antibody is afucosylated.

Preferably, the bispecific antibody comprises the “heavy chain for ErbB-2 binding” as depicted in the Sequence listing part 4 and the “heavy chain for ErbB-3 binding” as depicted in the Sequence listing part 4.

Preferably, the antigen binding sites of the bispecific antibody comprise a common light chain as defined herein, preferably a germline common light chain, preferably the rearranged germline human kappa light chain IgVκ1-39*01/IGJκ1*01 or a fragment or a functional derivative thereof (nomenclature according to the IMGT database worldwide web at imgt.org). The terms rearranged germline human kappa light chain IgVκ1-39*01/IGJκ1*01, IGKV1-39/IGKJ1, huVx1-39 light chain or in short huVx1-39 are used. The light chain can have 1, 2, 3, 4 or 5 amino acid insertions, deletions, substitutions or a combination thereof. The mentioned 1, 2, 3, 4 or 5 amino acid substitutions are preferably conservative amino acid substitutions, the insertions, deletions, substitutions or a combination thereof are preferably not in the CDR3 region of the VL chain, preferably not in the CDR1, CDR2 or CDR3 region or FR4 region of the VL chain. Preferably, the first antigen binding site and the second antigen binding site comprise the same light chain variable region, or rather, a common light chain. Preferably, the light chain variable region comprises a CDR1 having the sequence (RASQSISSYLN), a CDR2 having the sequence (AASSLQS), and a CDR3 having the sequence (QQSYSTPPT). Preferably, the light chain variable region comprises the common light chain sequence depicted FIG. 1.

Various methods are available to produce bispecific antibodies and are discussed in WO 2015/130173. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection.

The ratio of bispecific to other antibodies that are produced by the cell can be increased in various ways. Preferably, the ratio is increased by expressing not two different light chains but two essentially identical light chains in the cell. This concept is in the art also referred to as the “common light chain” method. When the essentially identically light chains work together with the two different heavy chains allowing the formation of variable domains with different antigen-binding sites and concomitant different binding properties, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. The art describes various ways in which such heterodimerization of heavy chains can be achieved. A preferred method is described in PCT application No. PCT/NL2013/050294 (WO 2013/157954 Al), which are incorporated herein by reference. Methods and means are disclosed for producing bispecific antibodies from a single cell, whereby means are provided that favor the formation of bispecific antibodies over the formation of monospecific antibodies.

For the purpose of clarity and a concise description features are described herein as part of the same or separate embodiments, however, it will be appreciated that the scope of the invention may include embodiments having combinations of all or some of the features described.

### BRIEF DESCRIPTION OF THE DRAWINGS

FIG. 1. Amino acid sequence of a) a common light chain amino acid sequence. b) common light chain variable region DNA sequence and translation (IGKVl-39/jkl).

c) Common light chain constant region DNA sequence and translation. d) IGKV1-39/jk5 common light chain variable region translation. e) V-region IGKV1-39A; f) CDR1, CDR2 and CDR3 of a common light chain.

FIG. 2. IgG heavy chains for the generation of bispecific molecules. a) CH1 region. b) hinge region. c) CH2 region. d) CH3 domain containing variations L351K and T366K (KK). e) CH3 domain containing variations L351D and L368E (DE).

FIG. 3. Amino acid alignments of variants of MF3178. Dots indicate the same amino acid as in MF3178 at that position. The CDR1, CDR2 and CDR3 sequences of MF3178 are in bold and underlined. The CDRs of the variants are at the corresponding positions.

FIG. 4. Sequence information on some nucleic acid and peptide molecules referred to in the application. Sequences 4A (ErbB-2 specific); Sequences 4B (ErbB-3 for an ErbB-3 binding arm in a bispecific antibody as described herein. Sequences 4D HER2-specific VH sequences and HER3-specific VH sequences.

FIG. 5. A depiction of the mechanism of action of an exemplary HER2/3 bispecific antibody administered in the invention and method of treatment. After selective docking of one arm of a bispecific antibody to HER2, binding of NRG1 to HER3 is prevented due to selective binding the second arm which in turn prevents phosphoinositide 3-kinase/protein kinase B (PI3K/AKT)-mediated cell proliferation/survival.

### EXAMPLES

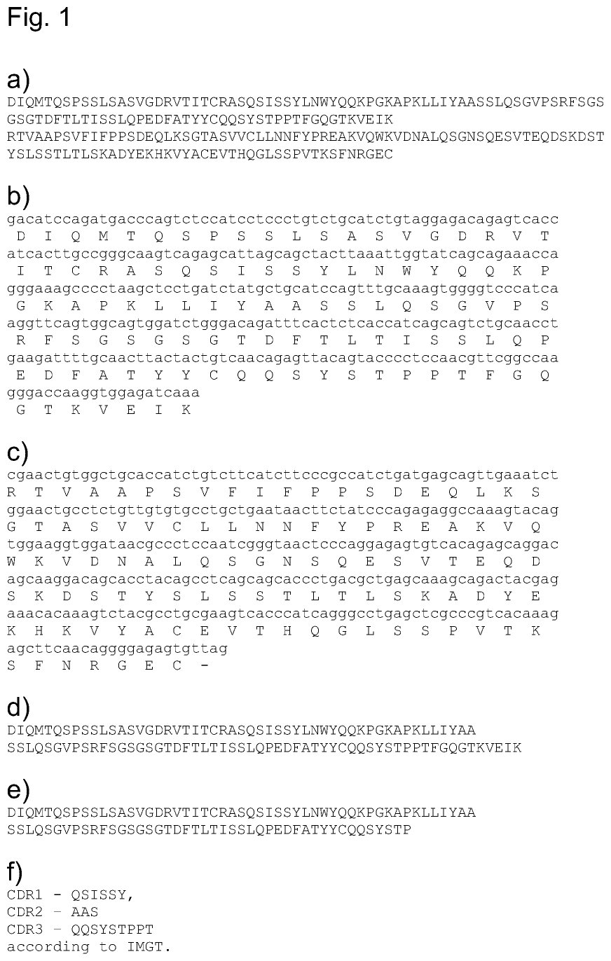

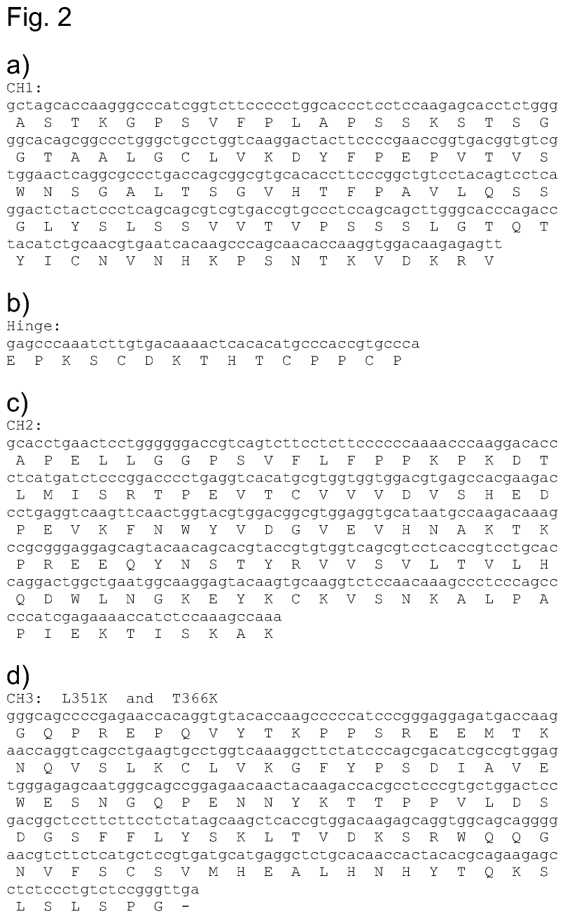

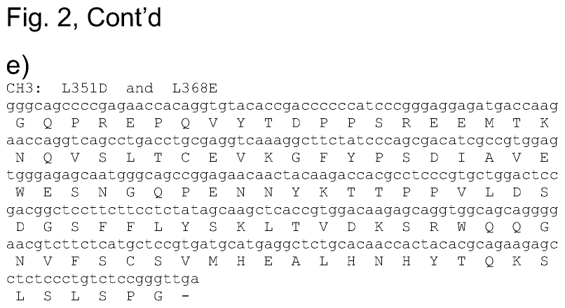

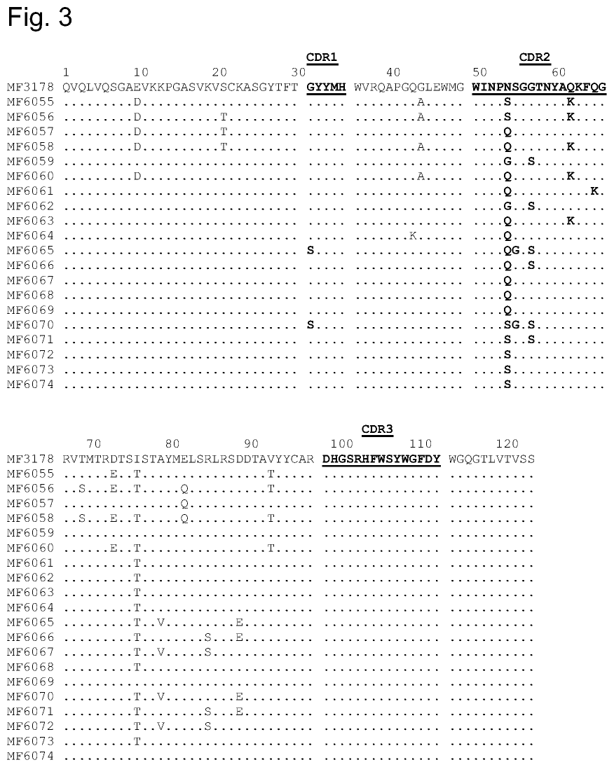

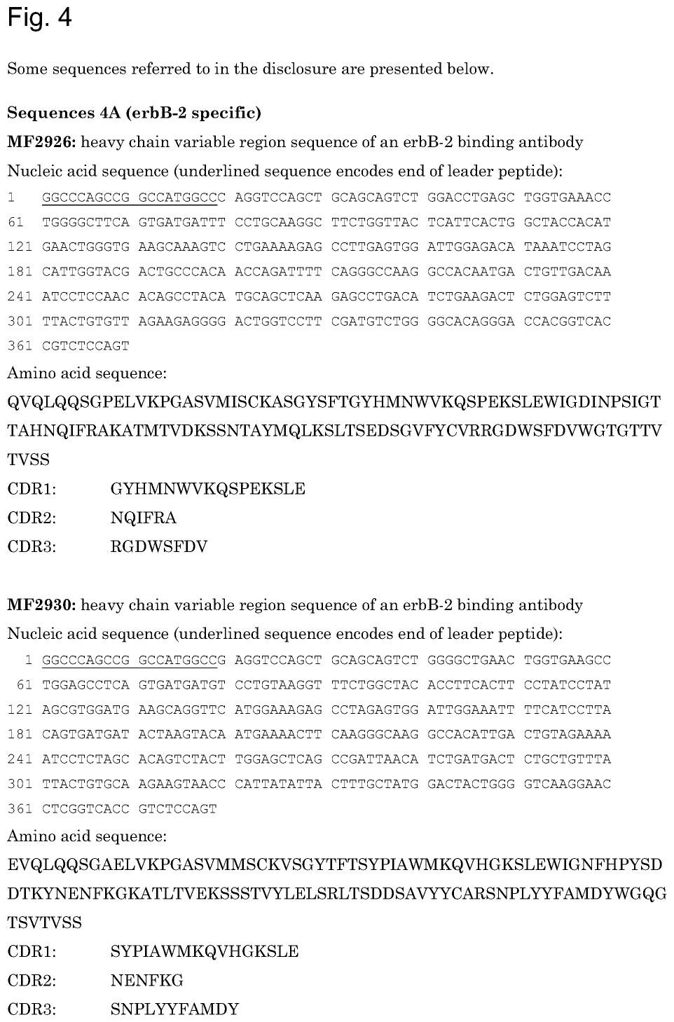

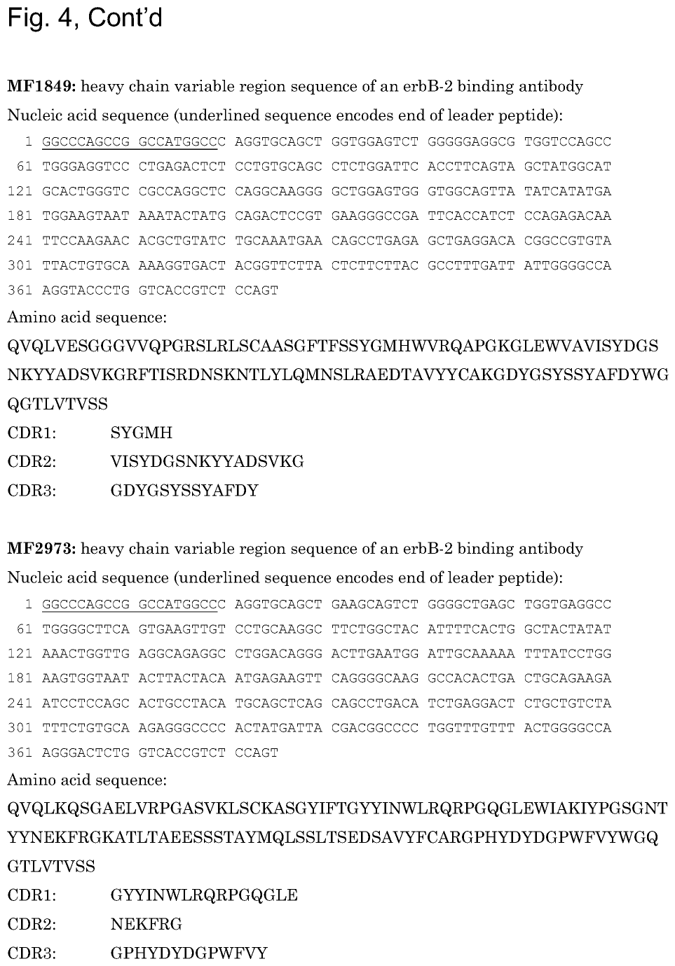

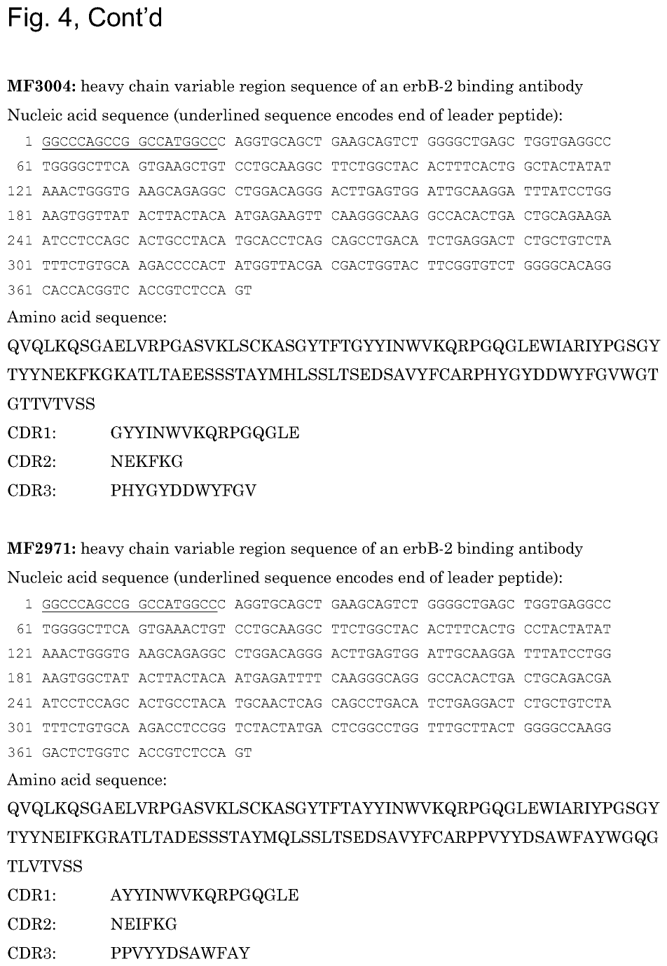

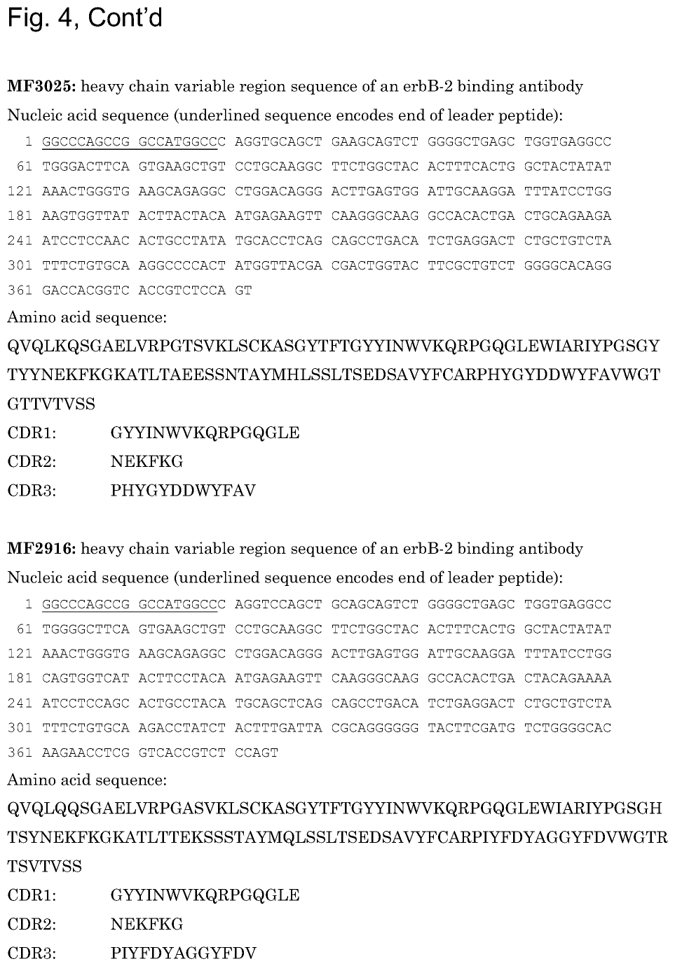

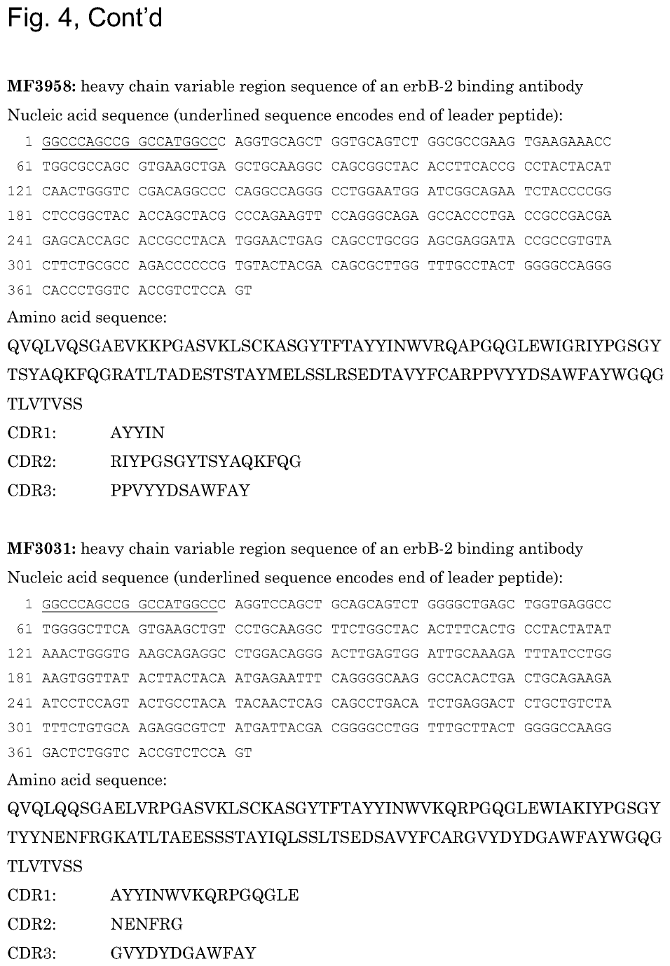

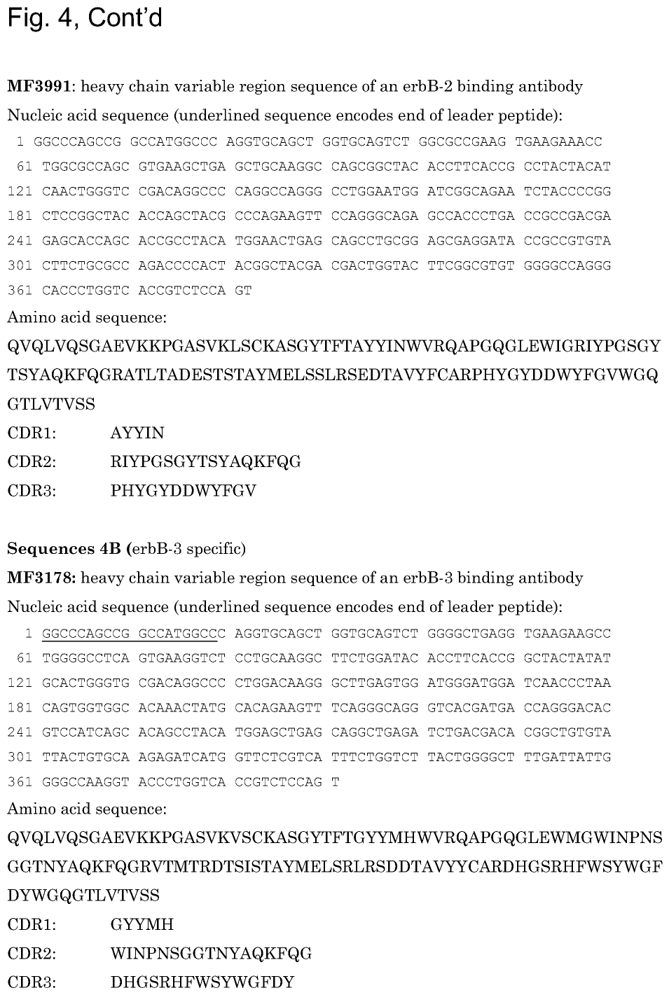

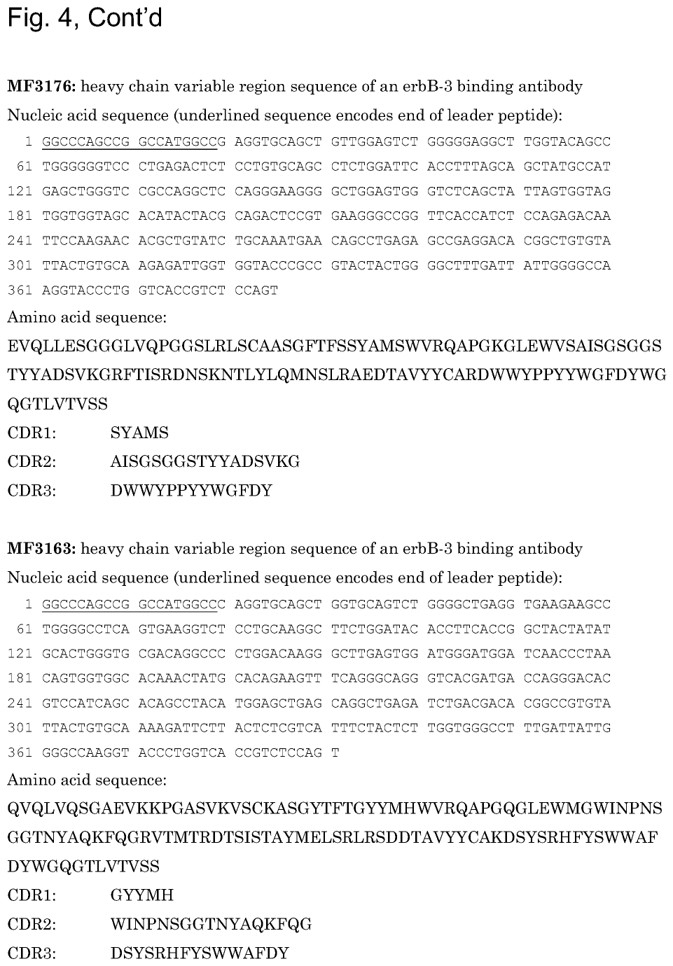

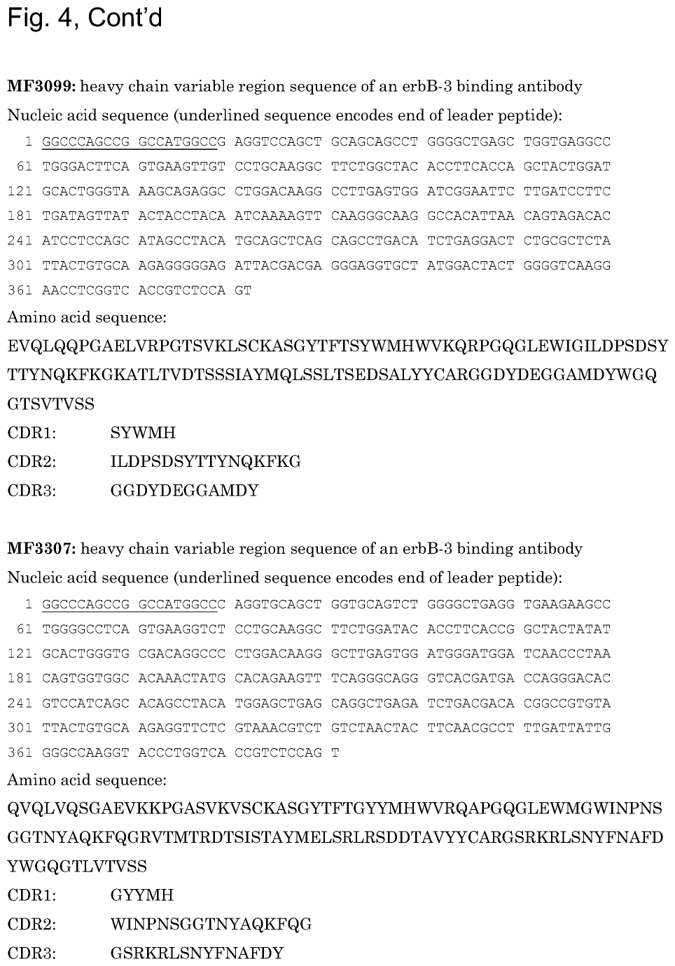

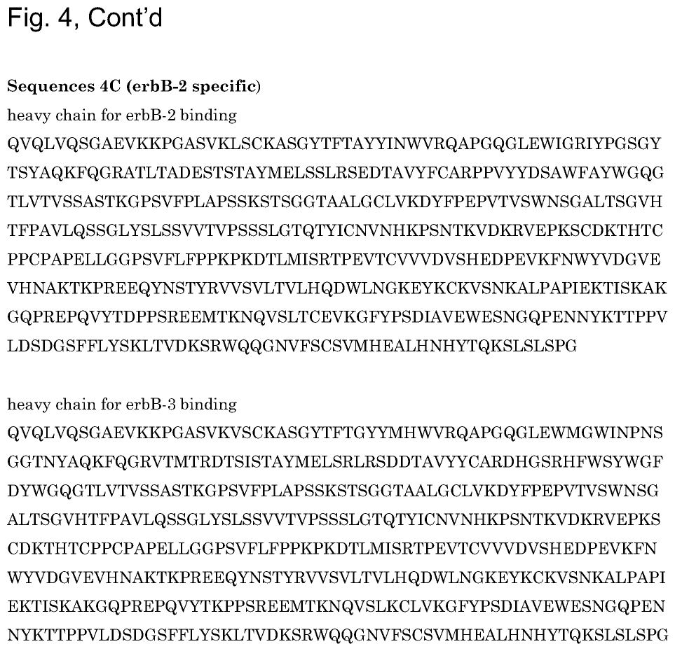

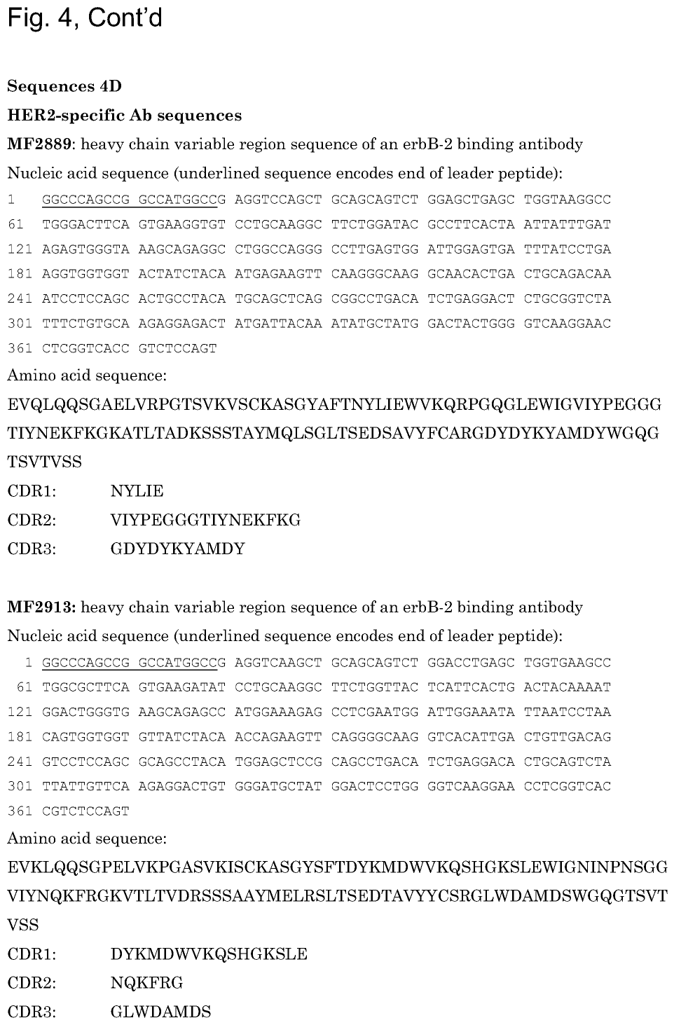

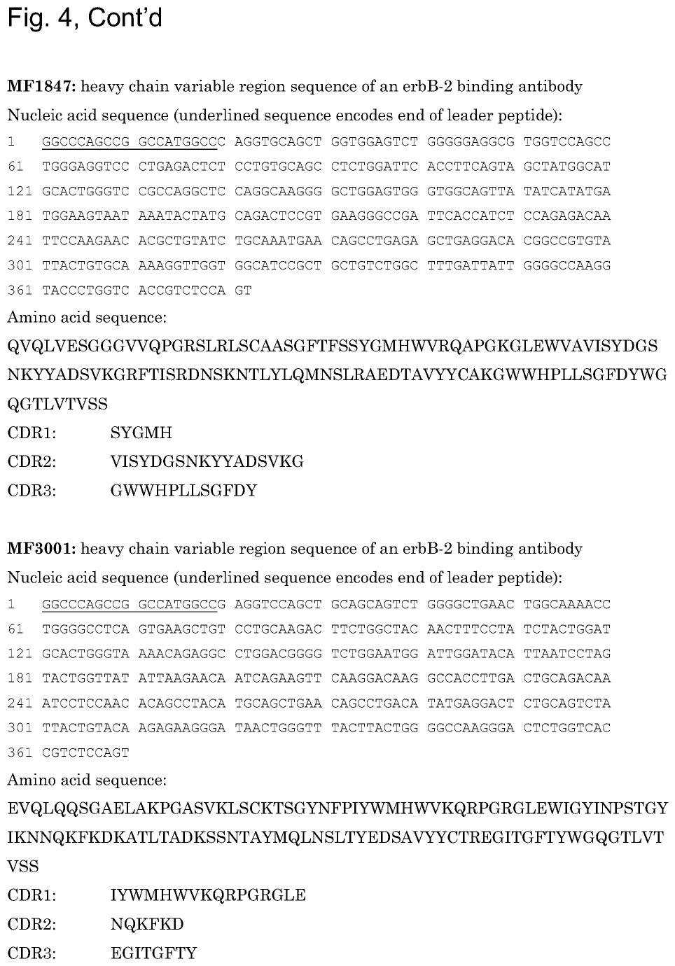

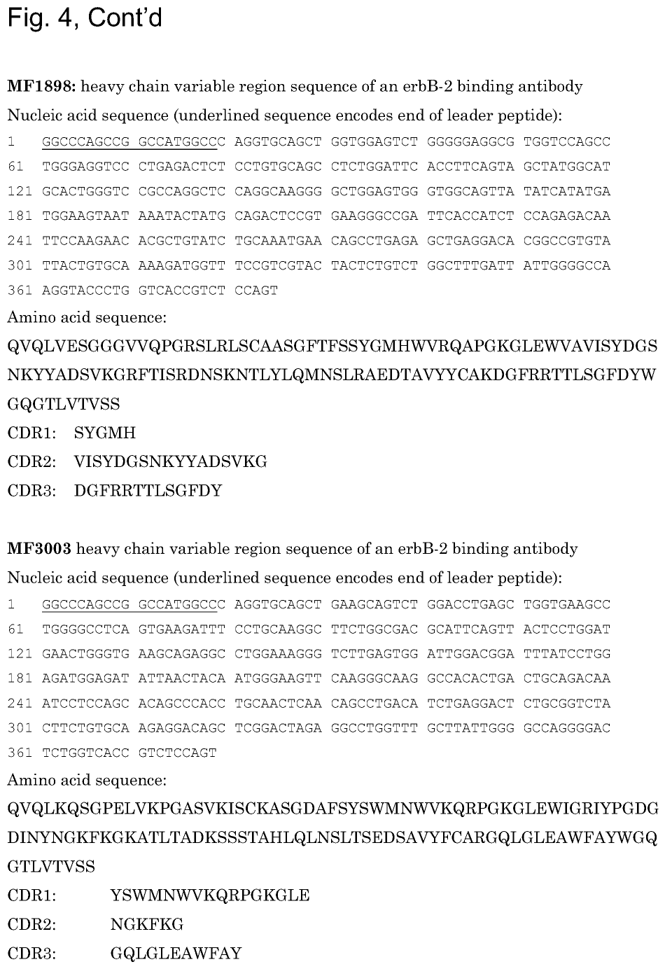

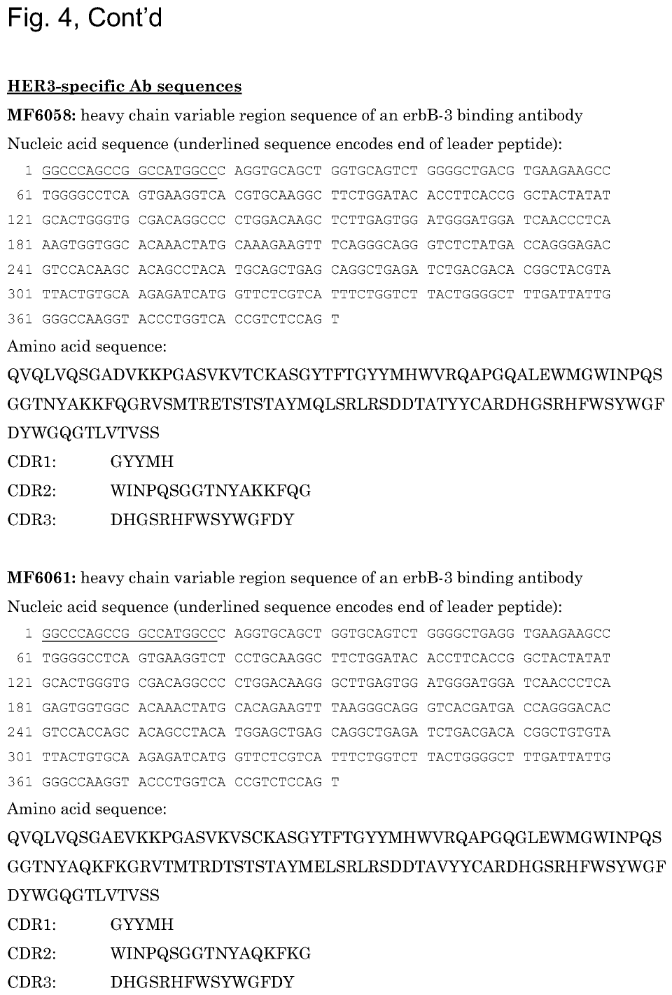

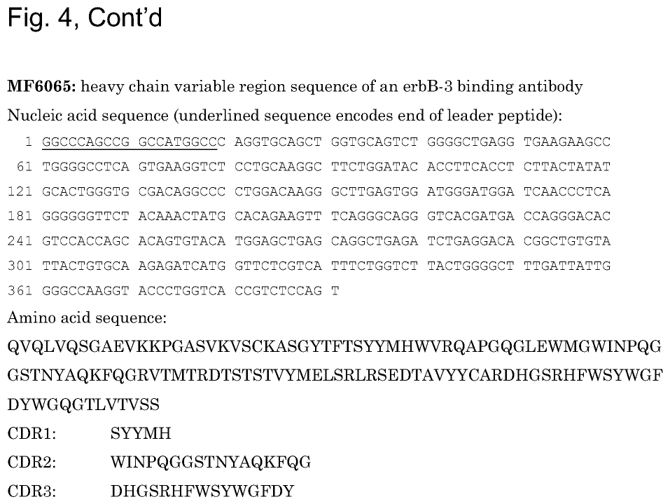

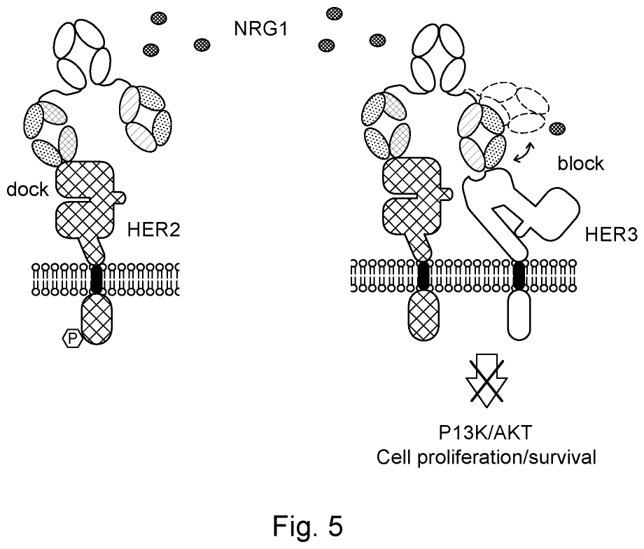

As used herein “MFXXXX” wherein X is independently a numeral 0-9, refers to a Fab comprising a variable domain wherein the VH has the amino acid sequence identified by the 4 digits depicted in FIG. 3 or 4. Unless otherwise indicated the light chain variable region of the variable domain typically has a sequence of FIG. 1a. The light chain in the examples has a sequence as depicted in FIG. 1b. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as W02013/157954) and FIGS. 2d and 2e). Bispecific antibodies in the examples have an Fc tail with a KK/DE CH3 heterodimerization domain, a CH2 domain and a CH1 domain as indicated in FIG. 2, a common light chain as indicated in figure la and a VHs as specified by the MF numbers.

### Example 1

### A Phase I/II Study of with the bispecific antibody MF3958 x MF3178, a full length IgG1 Bispecific Antibody Targeting HER2 and HER3, in Patients with Solid Tumors

### Study Duration

For the dose escalation part of the study (Part 1) 28 patients were recruited. Part 2 of the study is the dose expansion phase. The total duration of Part 2 is approximately 25 - 32 months; however, the actual duration is influenced by several variables, e.g., overall subject recruitment rate.

### Number of Patients

Twenty-eight (28) patients were enrolled in Part 1. For Part 2, at least 20 evaluable patients, and up to approximately 40, may be enrolled in the group advanced/ metastatic non-small cell lung cancer with invasive mucinous adenocarcinoma or documented NRG1 fusion; (NSCLC).

Patients who do not complete at least two cycles of study treatment due to other reasons than disease progression, are not evaluable for efficacy and are replaced in the respective group.

This Example describes Part 2 of the study.

### Study Objectives

Part 1

Objective

| | |
|---|---|
| Primary: | |
| Determination of the | Evaluation of adverse events (AEs) and dose |
| MTD and/or MRD of | limiting toxicities (DLT). |
| MF3958 × MF3178. | |
| Secondary: | |
| To characterize the safety | Frequency and nature of AEs/serious adverse |
| and tolerability of | events (SAEs). |
| MF3958 × MF3178. | |
| PK profile of | Assessment of PK variables, including total |
| MF3958 × MF3178. | exposure, maximum concentration (Cₘₐₓ) |
| | clearance, volume of distribution (V), |
| | volume of distribution at steady state |
| | (Vₛₛ), half-life (t_{1/2}), AUC₀₋ₜ |
| | (area under the concentration |
| | versus time curve from time zero to time t), |
| | AUC_{0-∞} (area under the concentration versus |
| | time curve), tₘₐₓ (time to reach maximum |
| | concentration). |
| Immunogenicity of | Incidence and serum titers of anti-drug |
| MF3958 × MF3178. | antibodies against MF3958 × MF3178. |
| Evaluation of anti-tumor | Anti-tumor activity and clinical benefit |
| response and CBR. | assessed by RECIST v1.1 determining |
| | objective overall response rate (ORR), |
| | duration of response (DOR), progression-free |
| | survival (PFS) and survival; CBR is defined |
| | as the proportion of patients in whom a |
| | complete response (CR) or partial response |
| | (PR) or stable disease (SD) is observed |
| | (where SD duration is a minimum of |
| | 12 weeks). |
| Exploratory (includes | |
| optional assessments): | |
| Presence of biomarkers and | Assessment of relevant tumor biomarkers |
| pharmacodynamic (PD) | and markers of MF3958 × MF3178 activity |
| responses to | in archival and/or fresh tumor biopsy |
| MF3958 × MF3178. | material and blood. The following candidate |
| | biomarkers are assessed: |
| | HER2, HER3, pHER2, pHER3 & |
| | heregulin; |
| | KRAS, NRAS, PIK3CA, BRAF mutation |
| | status (metastatic colorectal cancer |
| | (mCRC) patients only); |
| | circulating tumor deoxyribonucleic acid |
| | (DNA) and mutations in genes associated |
| | with HER2/HER3 signaling; |
| | phosphorylated molecules in the MAPK |
| | and AKT signaling pathway. |

Part 2

Objective

| | |
|---|---|
| Primary (safety): | |
| To characterize the safety and | Frequency and nature of AEs. |
| tolerability of MF3958 × | |
| MF3178. | |
| Primary (efficacy): | |
| To explore the relationships | Overall response rate (ORR), DOR, |
| between the anti-tumor | CBR (defined as the proportion of |
| activity of MF3958 × MF3178 | patients in whom a CR or PR is |
| and disease-related biomarkers | observed, or SD of a minimum |
| | duration of 12 weeks) per RECIST |
| | 1.1 as per local investigator's |
| | assessment. The relationship |
| | between anti-tumor activity and |
| | biomarkers including expression of |
| | HER2, HER3, and heregulin are |
| | explored, and serum biomarkers |
| | such as CA-125 (ovarian, |
| | endometrial) and CA-19-9 (gastric) |
| Secondary: | |
| PK profile of MF3958 × | Assessment of PK variables, including |
| MF3178. | total exposure, Cₘₐₓ, V, Vₛₛ, |
| | t_{1/2}, AUC₀₋ₜ, AUC_{0-∞}, tₘₐₓ. |
| | Population PK analysis |
| Immunogenicity of | Incidence and serum titers of anti- |
| MF3958 × MF3178. | drug antibodies against MF3958 × |
| | MF3178. |
| Evaluation of PFS and overall | |
| survival, duration of response | |
| Exploratory (includes | |
| optional assessments): | |
| Assessment of other relevant | The following candidate biomarkers |
| tumor biomarkers and markers | are assessed if sufficient sample is |
| of MF3958 × MF3178 activity | available: |
| in preferably fresh tumor | Tumor sample |
| sample/biopsy material or | pHER2, pHER3, HER2: HER3 |
| archival and blood. | dimerization; |
| | Heregulin and (depending on |
| | availability) mutations in cancer |
| | genes including those associated |
| | with HER2 and HERB |
| | Phosphorylated molecules in the |
| | MAPK and AKT signaling |
| | pathway. |
| | Heregulin-gene fusions |
| | Blood |
| | Fcgamma receptor polymorphism |
| | Circulating tumor DNA and |
| | mutation analysis in cancer genes |
| | including those associated with |
| | HER2/HER3 signaling; |
| | Circulating tumor cells and HER2 |
| | status |

### Study Design

This is a Phase I/II, open-label, multi-center, multi-national, dose escalation, single group assignment study to assess the safety, tolerability, PK, PD, immunogenicity and anti-tumor activity of MF3958 x MF3178.

The study is designed in 2 parts:

### Part 1

Part 1 of the study includes the investigation of nine dose levels: 40 mg, 80 mg, 160 mg in cohorts of 1 patient and 240 mg, 360 mg, 480 mg, 600 mg, 750 mg, and 900 mg in cohorts of 3 patients. MF3958 x MF3178 was initially given over approximately 60 minutes on Day 1 of a 3-week treatment cycle. During Part 1 the infusion duration was extended to 2 hours with the option of increasing it up to 4 hours to mitigate infusion-related reactions (IRRs).

No dose limiting toxicities (DLTs) were experienced at any of the dose levels. Three additional patients were dosed in each of the 600 mg and 750 mg cohorts in order to have sufficient PK information. As an MTD was not reached at the dose level of 900 mg, the Data Review Committee (DRC) for MF3958 x MF3178-CL01 decided to assign the dose level of 750 mg as the RP2D of the study, based on the cumulative safety, available PK data and PK simulations.

### Part 2

Part 2 includes a further characterization of the safety and tolerability of the selected dose level of MF3958×MF3178, as well as assessment of CBR, defined as the proportion of patients with a CR, PR or durable SD (SD for at least 12 weeks in duration), in expansion groups of selected patient populations.

A weekly dose regimen with a 4-week cycle is evaluated in newly recruited patients consisting of a flat dose of 400 mg weekly for the first 2 cycles, with an 800 mg loading dose for the initial administration. From cycle 3, MF3958 ×MF3178 is given at a dose of 400 mg weekly for 3 weeks followed by 1 week off. Mandatory pre-medication is administered to mitigate IRRs. However, corticosteroids are only mandatory prior to the loading dose of Day 1 of Cycle 1 and should only be used for subsequent infusions as per the investigator's discretion to manage IRRs.

Safety of the weekly schedule is reviewed during a run-in period after the first 5 patients treated have completed at least 2 treatment cycles. The DRC reviews all safety data with a focus on incidence of grade 3-4 toxicities, incidence and severity of IRRs, and compliance. If the DRC review concludes that toxicity is unacceptable, the Sponsor continues patient enrolment with the 3-week cycle dose regimen until a sufficient number of patients have been enrolled per cohort.

No within-patient dose escalation is permitted in Part 2.

Patient populations of interest to be assessed in Part 2 of the study are:
NSCLC with documented NRG1 fusions

At least 20 and up to approximately 40 patients may be enrolled in each Group (C-F) including a minimum of 10 patients per cohort treated with the weekly recommended dose. Previously closed cohorts may be reopened.

### Duration of Treatment

Patients in both Part 1 and 2 of the study may remain on treatment until disease progression, death, unacceptable toxicity or discontinuation for any other reason.

### Data Review Committee (DRC)

All dose escalation decisions in Part 1 were made by a DRC who convened to review all available safety data and PK data. The DRC participants included the Principal Investigators (or their representatives), the Sponsor's Medical Director, the study Medical Monitor, study Pharmacovigilance Physician, study Project Manager, study Statistician, and invited experts as required (such as clinical pharmacology expert).

In Part 2, the DRC reviews the data following completion of the safety run-in period for the weekly dose before expanding the weekly dose regimen in all subsequent patients.

### Study Assessments

The study consists of a molecular pre-screening assessment up to a 4-week (28-day) screening period, followed by sequential treatment cycles until treatment withdrawal or termination for any reason. The treatment cycle duration is 3 weeks (21 days) for patients treated at the initial recommended dose in Part 2, and 4 weeks (28 days) for patients treated at the weekly recommended dose in Part 2. All patients should attend an End of Treatment visit within 1 week after treatment cessation and a Final Study Visit 30 days after end of treatment or discontinuation from study.

Patients who have not progressed or withdrawn consent on completion of the Final Study Visit are followed up every 3 months for up to 2 years (approximately) to check their disease progression and/or survival status until the commencement of their next anti-cancer treatment.

Where ongoing evaluation of safety data and available PK, PD and anti-tumor activity data during the trial suggest that alternative dosing frequencies should be evaluated, or that other patient populations should be evaluated in Part 2, these modifications are clarified in a protocol amendment prior to commencing these evaluations.

### Molecular Pre-screening and Screening

Molecular pre-screening is performed in local laboratories qualified to perform molecular screening for NRG1 fusions. To initiate pre-screening, a patient must meet one of the following criteria:
Histological diagnosis of IMA and documented absence of EGFR/ALK alterations. Note: IMA patients who have not performed the pre-screening test for NRG1 fusion can enter the trial. OR
Pathological examination does not allow IMA diagnosis but the investigator suspects the IMA based on symptoms, imaging features (e.g. localized consolidation, multiple bilateral nodules or consolidations), non-smoker and documented absence of EGFR/ALK alterations.

The molecular pre-screening Informed Consent Form (ICF) must be signed by NSCLC patients identified for potential study participation before the fresh or archival tumor tissue is submitted for analysis for determination of NRG1 fusion status. Testing can be performed at any time of the natural history of the disease (e.g. at diagnosis, during the first line of therapy, at progression, etc) up to a maximum of one year prior to Cycle 1 Day 1. A fresh tumor sample (formalin-fixed paraffin-embedded; FFPE) or an archival tumor sample not older than 1 year, is required for the assessment of the presence of the NRG1 fusion. The sample should be submitted to a local laboratory qualified for testing by molecular profiling (PCR, next generation sequencing [DNA or RNA] or FISH) of NRG1 fusion status. Patients with a positive local NRG1 fusion result are then eligible to sign the main study ICF if they are willing and able to enter the main study.

### Main Informed Consent Form

The main study ICF must be signed by all patients prior to any screening procedures or assessments being conducted. The screening assessments are performed within 4 weeks prior to Cycle 1 Day 1, with the exception of the serum pregnancy test which must be conducted within 7 days of Cycle 1 Day 1. To be considered for screening, a baseline mandatory tumor sample, preferably a block, from fresh or archival tissue is requested. The sponsor indicates the preference for fresh tissue. Archival is acceptable and should have been taken within 2 years from screening other than for NSCLC which must be within 1 year. It should be noted that for NSCLC patients, the baseline biopsy for screening is still required even if a pre-screening biopsy sample is provided for pre-screening local testing of NRG1. Following completion of all required screening assessments and confirmation of all eligibility criteria the patient can begin dosing on Cycle 1 Day 1.

### Safety Assessments

Concurrent illnesses are captured at baseline; AEs and concomitant therapies are monitored throughout study participation. Safety assessments include reviewing Eastern Cooperative Oncology Group (ECOG) performance status, physical examination (including height and weight), vital signs and electrocardiograms (ECG). A cardiac function test of the Left Ventricular Ejection Fraction (LVEF) is also be carried out at Screening, end of Cycle 4 (or Cycle 5 Day 1), End of Study Visit, and at any time during the study if clinically indicated. Laboratory evaluations include clinical chemistry, hematology, coagulation tests, urinalysis and pregnancy testing. Note that a cytokine panel analysis was performed up until 1 Aug. 2017.

On all MF3958 ×MF3178 administration days, the patients must remain at the clinic for at least 60 minutes from the time of the end of infusion (longer where there are PK samples required) for observation and repeat vital signs prior to discharge from the clinic. Further additional safety assessments should be performed as clinically indicated and, if needed, duration of stay in clinic should be increased based on Investigator's judgment.

### Immunogenicity Assessment

Serum titers of anti-MF3958 ×MF3178 antibodies are measured on Day 1 at pre-dose for each of Cycles 1, 2, 3, 4 and then every fourth cycle thereafter (Cycle 8, 12, 16 etc), and at the End of Treatment Visit and the Final Study Visit with a window of −3 days prior to the MF3958 ×MF3178 administration.

### Pharmacokinetics Assessment

Part 1 and Part 2 initial recommended dose schedule: In Cycle 1, blood samples are collected for PK analysis on Day 1 at pre-dose, at end of infusion (EOI), and at 1, 2, 4, 8, 24 hours post EOI, then on Day 4 (or Day 3), Day 8 and Day 15. In Cycles 2-4, only a pre-dose and EOI blood sample is collected.

Part 2 weekly recommended dose schedule: In Cycle 1, blood samples are collected for PK analysis on Day 1 at pre-dose, EOI, 2, 4, 24 hours post EOI, then predose on Days 8 and 15, and predose and EOI on Day 22. In Cycles 2 and 3, a pre-dose and EOI blood sample is collected on Day 15. In Cycle 4 blood samples are collected pre-dose on Day 1, and pre-dose and EOI on Day 15. Every 2 cycles thereafter (Cycles 6, 8, 10 etc) a pre-dose blood sample is collected on Day 15.

### Tumor Assessment

Tumor assessment is evaluated according to RECIST version 1.1 per local investigator. Imaging is obtained at Screening and at the end of every 2 cycles of treatment for patients receiving the 3-week cycle regimen and every 6 weeks for patients receiving the 4-week cycle regimen.

Biomarker and Pharmacodynamics Assessments

A range of biomarker and pharmacodynamic tests are performed on archived and/or fresh tumor sample material and/or blood (liquid biopsy), depending on availability of archived or existincl tumor tissue, consent for further tumor samples, and consent for specific biomarker testing.

The following candidate biomarkers are assessed in case sufficient sample is available:
HER2, HER3, HER2:HER3 dimerization, phosphorylated HER2 (pHER2) and HER3 (pHER3) and heregulin;
Circulating plasma tumor DNA (ctDNA) and tumor sample DNA (depending on availability) are used to examine mutations in cancer genes including those associated with HER2 and HER3 signaling
Phosphorylated molecules in the MAPK and AKT signaling pathway;
Fcgamma receptor polymorphism;
Circulating tumor cells for HER2;
Heregulin-gene fusions

No germ line DNA assessment is included (except for Fcgamma receptor polymorphism).

At baseline the patient is requested to provide a mandatory tumor sample tissue, preferably a block, which could be from fresh or archival tissue. The sponsor indicates the preference for fresh tissue. Archival is acceptable and should have been taken within 2 years from screening other than for NSCLC which must be within 1 year. In addition the patient is requested optionally to provide a tumor sample/biopsy at the end of Cycle 4 and optionally at the End of Treatment Visit.

Blood samples are also taken at these time points for the purpose of liquid biopsy testing.

### Eligibility Criteria

The study enrolls patients with NSCLC.

### General Inclusion Criteria for Part 2

1. Age 18 years or older;
2. At least one measurable lesion according to RECIST v1.1;
3. Performance status of ECOG 0 or 1;
4. Estimated life expectancy of at least 12 weeks;
5. Toxicities incurred as a result of previous anti-cancer therapy resolved to ≤Grade 1 (as defined by NCI CTCAE v4.03), except for alopecia, lymphopenia assessed as non-clinically significant, Grade 2 sensory neurotoxicity;
6. At least a 4-week interval between the last received radiotherapy and the first scheduled day of dosing with MF3958 ×MF3178 (with the exception of up to 1×8 Gy for pain palliation);
7. Complete recovery from major surgery (stable and <Grade 2 toxicity acceptable);
8. Laboratory values at Screening:
   a. Absolute neutrophil count >1.5×10⁹/L without colony stimulating factor support;
   b. Platelets ≥100×10⁹/L;
   c. Hemoglobin ≥9 g/dL or ≥2.2 mmol/L (not transfusion dependent);
   d. Total bilirubin <1.5 times the upper limit of normal (ULN) (unless due to Gilbert's syndrome);
e. AST (SGOT) ≤2.5×ULN; ALT (SGPT) ≤2.5×ULN; ≤5×ULN for patients with advanced solid tumors with liver metastases; patients with confirmed bony metastases are permitted on study with isolated elevations in ALP >5×ULN;
f. Serum creatinine ≤1.5×ULN or estimated glomerular filtration rate (GFR) of >50 mL/min based on the Cockroft-Gault formula;
g. Coagulation function (INR and aPTT ≤1.5 ULN, unless on therapeutic anticoagulants)
h. Urine protein ≤2+(as measured by dipstick) or ≤100 mg/24 hours urine;

9. Able to provide at baseline a mandatory tumor biopsy sample (FFPE), preferably a block, from fresh (preferred) or archival tissue. Archival tissue must be collected within 2 years before screening, other than for NSCLC which must be within 1 year.
10. Negative pregnancy test results available as defined by urine or blood human chorionic gonadotropin (hCG) test during Screening and within 7 days of Cycle 1, Day 1 in women of childbearing potential (defined as women ≤50 years of age or history of amenorrhea for ≤12 months prior to study entry);
11. Sexually active male and female patients of childbearing potential must agree to use an effective method of birth control (e.g., barrier methods with spermicides, oral or parenteral contraceptives and/or intrauterine devices) during the entire duration of the study and for 6 months after final administration of MF3958×MF3178. Note that sterility in female patients must be confirmed in the patients' medical records and be defined as any of the following: surgical hysterectomy with bilateral oophorectomy, bilateral tubular ligation, natural menopause with last menses >1 year ago; radiation induced oophorectomy with last menses >1 year ago; chemotherapy induced menopause with 1 year interval since last menses;
12. Ability to give written, informed consent prior to any study-specific Screening procedures, with the understanding that the consent may be withdrawn by the patient at any time without prejudice;
13. Capable of understanding the mandated and optional protocol requirements, is willing and able to comply with the study protocol procedures and has signed the main informed consent document. For any optional biopsy sampling (tissue and/or blood) and long-term sample storage, additional consent is required;
14. Patient with metastatic cancer who has disease progression after having received treatment with all available therapies known to convey clinical benefit.
15. Unresectable or metastatic NSCLC meeting one of the following conditions:
   Biopsy-proven invasive mucinous adenocarcinoma (IMA). Note: IMA patients who have not performed the pre-screening test for NRG1 fusion can enter the trial. OR
   NSCLC with documented NRG1 fusion determined at in a qualified local laboratory by molecular profiling using methods such as PCR, next generation sequencing [DNA or RNA] or FISH in patients with no known driver mutations or fusions in EGFR/ALK genes. 16. Documented disease progression by investigator assessment on at least one line of standard therapy in the locally advanced or metastatic setting.

### Statistical Analysis

### Part 1 and Part 2

Anti-tumor and clinical benefit variables are summarized descriptively for each group in Part 2. Where appropriate, variables are presented in terms of absolute and relative change from baseline. Categorical data is presented as percentages and frequency tabulations.

Where appropriate, data from those patients who receive what becomes identified as the MTD or the MRD during Part 1, and those receiving the same dose in Part 2, may be combined and summarized, as well as being summarized independently.

The frequency and nature of serious and non-serious AEs is assessed in absolute and relative frequencies and coded according to MedDRA medical dictionary.

### Part 1

Data evaluation is descriptive in nature. Patient demographics, disease characteristics and pharmacokinetic and pharmacodynamic variables are summarized at each dose level. The frequency and nature of DLTs are also summarized at each dose level.

### Part 2

With N=20 per cohort in Part 2, clinically meaningful observed correlation coefficients of at least 0.38 would be distinguishable from zero with 95% confidence; lesser, non-clinically meaningful observed correlations would not be distinguishable from zero. Hence 20 subjects per cohort in Part 2 is considered sufficient to explore the relationship between the anti-tumor activity of MF3958 x MF3178 and disease related biomarkers.

In the event that signs of clinical activity are seen, additional patients up to a total of approximately 40 may be recruited. With 40 patients, true clinical response rates of, for example, 10% to 50% can be estimated with reasonable precision of approximately ±5% to ±8%.

PK parameters are summarized for each cohort in Part 1 and each tumor group in Part 2. Arithmetic and geometric means are provided in addition to medians, range, SD and %CV. AUC is calculated according to the trapezoid rule. Serum concentration profiles against time are plotted for each group.

### Example 2

Treatment with a HER2-HER3 bispecific antibody of a patient that received and whose cancer progressed with afatinib as a prior-treatment

An 38-year-old female diagnosed with NSCLC having histology invasive mucinous adenocarcinoma was treated with MF3958×MF3178. This study shows that MF3958×MF3178 stabilizes the tumor in terms of size or lesions. The treatment prevents further tumor growth

### Tumor Molecular Profiling

Analysis of tumor tissue obtained from lung showed an SDC4-NRG1 fusion. In addition the tumor genome showed absence of mutations in EGFR, KRAS, EGFR, cKIT-BRCA1-2, MET, ROS, RET, ALK.

The analysis was performed using Oncomine, which is a targeted DNA sequencing method designed for cancer research and allows the analysis of multiple biomarkers, including fusions, insertion/deletions (indels), single nucleotide variants, and copy number variations. The outcome was verified by RNA seq and Anchored Multiplex PCR (Archer).

### Prior Treatment

The patient received adjuvant treatment with cisplatin, carboplatin, pemetrexed from during 4 months. Within 5-6 months the patient presented bilateral relapse in the lung. Subsequently, the patient received treatment with afatinib for approximately 11 months until new progression of the cancer was detected. The patient entered the study within two months after stopping the afatinib treatment.

### Clinical Status at Study Entry

At the entry of the study this patient represented a NSCLC with metastatic tumor extension in lung and pleura.

The patient presented with a good general condition (performance status (ECOG) of 1). ECOG Scale of Performance Status describes a patient's level of functioning in terms of their ability to care for themselves, daily activity, and physical ability. A ECOG score of 1 reflects a patients restricted in physically strenuous activity but ambulatory and able to carry out work of a light or sedentary nature.

### Treatment with the Bispecific Antibody MF3958×MF3178

Treatment was given with bispecific antibody MF3958×MF3178 on a weekly regimen. The first dose was administered with premedication. In total 8 cycles were administered, during a study period of 8 months.

A weekly dose regimen with a 4-week cycle consists of a flat dose of 400 mg weekly for the first 2 cycles, with an 800 mg loading dose for the initial administration. From cycle 3, MF3958×MF3178 is given at a dose of 400 mg weekly for 3 weeks followed by 1 week off. Mandatory pre-medication is administered to mitigate IRRs. However, corticosteroids are only mandatory prior to the loading dose of Day 1 of Cycle 1 and should only be used for subsequent infusions as per the investigator's discretion to manage IRRs.

### Safety

The patient did not experience any severe episode of study drug related toxicity.

### Efficacy

The disease was assessed with measurable disease in the lung by using CT-scan. Anti-tumor activity and clinical benefit assessed by RECIST v1.1 determining objective overall response rate (ORR), duration of response (DOR), progression-free survival (PFS) and survival. Four tumor assessments reported stable disease (RECIST v1.1). The study shows that MF3958×MF3178 stabilizes the tumor in terms of size or lesions. The treatment prevents further tumor growth.

### Example 3

Post-chemotherapy and afatinib treatment with a HER2-HER3 bispecific antibody in patients with NRG1 fusions.

Alternatively to the dosing regimen of Example 2, medication can be provided in a bi-weekly dosing schedule as follows:

Bi-weekly schedule consisting of 750 mg of MF3958×MF3178 over four hours for the first infusion and then over two hours for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

### Example 4

Post-chemotherapy and afatinib treatment with a HER2-HER3 bispecific antibody in patients with NRG1 fusions.

Alternatively to the dosing regimen of Example 3, medication can be provided in a tri-weekly dosing schedule as follows:

Tri-weekly schedule consisting of 750 mg of MF3958×MF3178 over four hours for the first infusion and then over two hours for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

1. A bispecific antibody that comprises a first antigen-binding site that binds an extracellular part of ErbB-2 and a second antigen-binding site that binds an extracellular part of ErbB-3 for use in the treatment of a subject that has an ErbB-2 and ErbB-3 positive cancer, cells of which cancer comprise a neuregulin-1 (NRG1) fusion gene comprising at least a portion of the NRG1-gene fused to a sequence from a different chromosomal location, and which cancer in said subject has progressed after having received a prior treatment with a monospecific bivalent antibody comprising antigen-binding sites that bind an extracellular part of ErbB-2 or an extracellular part of ErbB-3, or a prior treatment with a tyrosine kinase inhibitor (TKI) of ErbB-2 or chemotherapy or with a combination thereof.

2. The bispecific antibody for use of claim 1, wherein the subject is human subject.

3. The bispecific antibody for use of claim 1 or claim 2, wherein the TKI is preferably one or more of lapatinib, canertinib, neratinib, tucatinib, CP-724714, tarloxitinib, mubritinib, afatinib, varlitinib, and dacomitinib. preferably afatinib.

4. The bispecific antibody for use of any one of claims 1-3, wherein the TKI is afatinib.

5. The bispecific antibody for use of claim 1, wherein the monospecific bivalent antibody comprising antigen-binding sites that bind an extracellular part of ErbB-3 comprises patritumab, seribantumab, lumretuzumab, elgemtumab, GSK2849330, KTN3379 or AV-203.

6. The antibody for use of any one of claims 1-5, wherein the NRG1 fusion gene comprises at least the 3′ end of the NRG1-gene fused to a 5′ sequence from a different chromosomal location.

7. The antibody for use of any one of claims 1-6, wherein the cancer cell is driven by the NRG1-fusion gene.

8. The antibody for use of any one of the preceding claims wherein the cancer is a recurrent cancer or a metastasized cancer.

9. The antibody for use of any of the preceding claims, wherein the cancer is a breast cancer, an ovarian cancer, a lung cancer such as non-small cell lung cancer (NSCLC), pancreatic ductal adenocarcinoma, renal cell adenocarcinoma, sarcoma, gall bladder cancer, bladder cancer, cholangiocarcinoma, head and neck cancer, prostate cancer, uterine cancer, sinonasal teratocarcinosarcoma, colorectal adenocarcinoma, a liver cancer or colorectal cancer.

10. The antibody for use of any of the preceding claims, wherein the cancer is a non-small cell lung cancer.

11. The antibody for use of any of the preceding claims, wherein the NRG1-fusion gene expresses a protein that comprises an NRG1 EGF-like domain.

12. The antibody for use of any of the preceding claims, wherein the NRG1-fusion gene is a fusion of NRG1 and a gene on human chromosome 8.

13. The antibody for use of claim 10, wherein the gene on human chromosome 8 encodes an excreted protein or a cellular membrane associated protein.

14. The antibody for use of any of the preceding claims, wherein the NRG1 fusion gene is a fusion of the 3′ end of the NRG1-gene with the 5′ sequence of one of the genes selected from the group consisting of ADAM9, AKAP13, APP, ATP1B1, BMPR1B, CCND1, CD44, CD74, CDH1, CDH6, CDK1, CLU, COX10-AS1, DIP2B, DOC4, DPYSL2, FOXA1, GDF15, HMBOX1, KIF13B, MCPH1, MDK, MRPL13, NOTCH2, PARP8, PDE7A, POMK, RAB2IL1, RAB3IL1, RBPMS, ROCK1, SDC4, SETD4, SLC3A2, SLC4A4, SMAD4, STAU3, THAP7, THBS1, TNC, TNFRSF10B, TNKS, TSHZ2, VAMP2, VTCN1, WHSC1L1, WRN and ZMYM2, preferably an SDC4; ATP1B1; or a CD74 fusion, more preferably an SDC4-NRG1 fusion or the NRG1 fusion gene is a fusion of the 5′ end of the NRG1-gene with the 3′ sequence of the fusion partner, such as genes selected from the group consisting of FOXA1, PMEPA1, RAD51, and STMN2.

15. The antibody for use of any of the preceding claims, wherein the NRG1 fusion gene is a fusion of the 3′ end of the NRG1-gene with the 5′ sequence of the gene SDC4.

16. The antibody for use of any of the preceding claims, wherein said first antigen-binding site binds domain I of ErbB-2 and said second antigen-binding site binds domain III of ErbB-3, preferably wherein the affinity of the first antigen-binding site for ErbB-2 is lower than the affinity of the second antigen-binding site for ErbB-3.

17. The antibody for use according to claim 16, wherein said antibody comprises i) at least the CDR1, CDR2 and CDR3 sequences of an ErbB 2 specific heavy chain variable region selected from the group consisting of MF2926, MF2930, MF1849;
MF2973, MF3004, MF3958, MF2971, MF3025, MF2916, MF3991, MF3031, MF2889, MF2913, MF1847, MF3001, MF3003 and MF1898 or wherein said antibody comprises CDR sequences that differ in at most 3 amino acids, preferably in at most 2 amino acids, preferably in at most 1 amino acid from the CDR1, CDR2 and CDR3 sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) at least the CDR1, CDR2 and CDR3 sequences of an ErbB 3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

18. The antibody for use according to claim 16 wherein said antibody comprises
i) an ErbB 2 specific heavy chain variable region sequence selected from the group consisting of the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 and MF1898, or wherein said antibody comprises a heavy chain variable region sequence that differs in at most 15 amino acids from the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) an ErbB 3 specific heavy chain variable region sequence selected from the group consisting of the heavy chain variable region sequences of MF3178; MF3176;
MF3163; MF3099; MF3307; MF6055; MF6056; MF6057; MF6058; MF6059;
MF6060; MF6061; MF6062; MF6063; MF6064; MF 6065; MF6066; MF6067;
MF6068; MF6069; MF6070; MF6071; MF6072; MF6073 and MF6074, or wherein said antibody comprises a heavy chain variable region sequence that differs in at most 15 amino acids from the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

19. The antibody for use of claims 16-18, wherein the antibody comprises at least the CDR1, CDR2 and CDR3 sequences of the ErbB 2 specific heavy chain variable region MF3958 and the antibody comprises at least the CDR1, CDR2 and CDR3 sequences of the ErbB 3 specific heavy chain variable region MF3178.

20. The antibody for use of any of the preceding claims, wherein the variable domain that comprises said first antigen binding site and the variable domain that comprises said second antigen binding site comprise a light chain variable region comprising the IgVK1-39 gene segment, most preferably the rearranged germline human kappa light chain IgVK1-39*01/IGJK1*01.

21. The antibody for use of any of the preceding claims, wherein the variable domain that comprises said first antigen binding site and the variable domain that comprises said second antigen binding site comprise a light chain variable region comprising a CDR1 having the sequence (RASQSISSYLN), a CDR2 having the sequence (AASSLQS), and a CDR3 having the sequence (QQSYSTPPT), which numbering is according to KABAT.

22. The antibody for use of any of the preceding claims, wherein the chemotherapy comprises gemcitabine, capecitabine, carboplatin, a taxane, such as docetaxel or paclitaxel, 5-fluorouracil (with or without radiotherapy), vinorelbine, mitoxantrone, vinblastine, cisplatin (or pemetrexed), oxaliplatin, carboplatin, ifosfamide, mytomycine C, vindesine, etoposide, Folfox (i.e. a combination of 5-fluorouracil, leucovorin, and oxaliplatin) or Folfiri (i.e. a combination of leucovorin, 5-fluorouracil and irinotecan),Folfirinox (a combination of leucovorin, 5-fluorouracil, irinotecan and oxaliplatin) or any combination thereof.

23. A method of treating a subject that has an ErbB-2 and ErbB-3 positive cancer, cells of which cancer comprise an NRG1 fusion gene comprising at least a portion of the NRG1-gene fused to a sequence from a different chromosomal location, and which subject is a pretreatment cancer subject that had received a previous ErbB-2 or ErbB-3 targeted tumor treatment, the method comprising administering to the subject in need thereof a bispecific antibody that comprises a first antigen-binding site that binds an extracellular part of ErbB-2 and a second antigen-binding site that binds an extracellular part of ErbB-3.


SEQUENCE LISTING (170 sequences)

SEQ ID NO: 1 - protein, 11 aa, Artificial Sequence
  light chain CDR1
RASQSISSYLN

SEQ ID NO: 2 - protein, 7 aa, Artificial Sequence
  light chain CDR2
AASSLQS

SEQ ID NO: 3 - protein, 9 aa, Artificial Sequence
  light chain CDR3
QQSYSTPPT

SEQ ID NO: 4 - protein, 6 aa, Artificial Sequence
  light chain CDR
QSISSY

SEQ ID NO: 5 - protein, 3 aa, Artificial Sequence
  light chain CDR
AAS

SEQ ID NO: 6 - protein, 214 aa, Artificial Sequence
  common light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 321 bp, Artificial Sequence
  common light chain
  CDS (1)..(321)
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 8 - protein, 107 aa, Artificial Sequence
  Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 9 - DNA, 324 bp, Artificial Sequence
  IGKV1-39/jk5 common light chain
  CDS (1)..(324)
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 10 - protein, 107 aa, Artificial Sequence [sequence omitted]

SEQ ID NO: 11 - protein, 107 aa, Artificial Sequence; identical to SEQ ID NO: 8
  CH3 domain containing variations L351K and T366K

SEQ ID NO: 12 - protein, 95 aa, Artificial Sequence
  V-region IGKV1-39A
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 13 - DNA, 294 bp, Artificial Sequence
  IgG heavy chain CH1
  CDS (1)..(294)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 14 - protein, 98 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 15 - DNA, 45 bp, Artificial Sequence
  IgG heavy chain hinge
  CDS (1)..(45)
gagcccaaatcttgtgacaaaactcacacatgcccaccgtgccca

SEQ ID NO: 16 - protein, 15 aa, Artificial Sequence
  Synthetic Construct
EPKSCDKTHTCPPCP

SEQ ID NO: 17 - DNA, 330 bp, Artificial Sequence
  IgG heavy chain CH2
  CDS (1)..(330)
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 18 - protein, 110 aa, Artificial Sequence
  Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 19 - DNA, 321 bp, Artificial Sequence
  IgG heavy chain CH3 L351K and T366K
  CDS (1)..(321)
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 20 - protein, 106 aa, Artificial Sequence
  Synthetic Construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - DNA, 321 bp, Artificial Sequence
  IgG heavy chain CH3 L351D and L368E
  CDS (1)..(321)
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 22 - protein, 106 aa, Artificial Sequence
  Synthetic Construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 124 aa, Artificial Sequence
  MF3178
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 24 - protein, 124 aa, Artificial Sequence
  MF6055
QVQLVQSGADVKKPGASVKVSCKASGYTFTGYYMHWVRQAPGQALEWMGWINPSSGGTNYAKKFQGRVTMTRETSTSTAYMELSRLRSDDTATYYCARDHGSRHFWSYWGFDYWGQGTLVTVSS

SEQ ID NO: 25 - protein, 124 aa, Artificial Sequence
  MF6056
QVQLVQSGADVKKPGASVKVTCKASGYTFTGYYMHWVRQAPGQALEWMGWINPSSGGTNYAKKFQGRVSMTRETSTSTAYMQLSRLRSDDTATYYCARDHGSRHFWSYWGFDYWGQGTLVTVSS

SEQ ID NO: 26 - protein, 124 aa, Artificial Sequence
  MF6057
QVQLVQSGADVKKPGASVKVTCKASGYTFTGYYMHWVRQAPGQGLEWMGWINPQSGGTNYAQKFQGRVTMTRDTSISTAYMQLSRLRSDDTAVYYCARDHGSRHFWSYWGFDYWGQGTLVTVSS

SEQ ID NO: 27 - protein, 124 aa, Artificial Sequence
  MF6059
QVQLVQSGAEVKKPGASVKVSCKASGYTFTGYYMHWVRQAPGQGLEWMGWINPGSGSTNYAQKFQGRVTMTRDTSISTAYMELSRLRSDDTAVYYCARDHGSRHFWSYWGFDYWGQGTLVTVSS

SEQ ID NO: 28 - protein, 124 aa, Artificial Sequence
  MF6060
QVQLVQSGADVKKPGASVKVSCKASGYTFTGYYMHWVRQAPGQALEWMGWINPQSGGTNYAKKFQGRVTMTRETSTSTAYMELSRLRSDDTATYYCARDHGSRHFWSYWGFDYWGQGTLVTVSS

SEQ ID NO: 29 - protein, 124 aa, Artificial Sequence
  MF6062
QVQLVQSGAEVKKPGASVKVSCKASGYTFTGYYMHWVRQAPGQGLEWMGWINPGSGSTNYAQKFQGRVTMTRDTSTSTAYMELSRLRSDDTAVYYCARDHGSRHFWSYWGFDYWGQGTLVTVSS

SEQ ID NO: 30 - protein, 124 aa, Artificial Sequence
  MF6063
QVQLVQSGAEVKKPGASVKVSCKASGYTFTGYYMHWVRQAPGQGLEWMGWINPQSGGTNYAKKFQGRVTMTRDTSTSTAYMELSRLRSDDTAVYYCARDHGSRHFWSYWGFDYWGQGTLVTVSS

SEQ ID NO: 31 - protein, 124 aa, Artificial Sequence
  MF6064
QVQLVQSGAEVKKPGASVKVSCKASGYTFTGYYMHWVRQAPGKGLEWMGWINPQSGGTNYAQKFQGRVTMTRDTSTSTAYMELSRLRSDDTAVYYCARDHGSRHFWSYWGFDYWGQGTLVTVSS

SEQ ID NO: 32 - protein, 124 aa, Artificial Sequence
  MF6066
QVQLVQSGAEVKKPGASVKVSCKASGYTFTGYYMHWVRQAPGQGLEWMGWINPQSGSTNYAQKFQGRVTMTRDTSTSTAYMELSSLRSEDTAVYYCARDHGSRHFWSYWGFDYWGQGTLVTVSS

SEQ ID NO: 33 - protein, 124 aa, Artificial Sequence
  MF6067
QVQLVQSGAEVKKPGASVKVSCKASGYTFTGYYMHWVRQAPGQGLEWMGWINPQSGGTNYAQKFQGRVTMTRDTSTSTVYMELSSLRSDDTAVYYCARDHGSRHFWSYWGFDYWGQGTLVTVSS

SEQ ID NO: 34 - protein, 124 aa, Artificial Sequence
  MF6068
QVQLVQSGAEVKKPGASVKVSCKASGYTFTGYYMHWVRQAPGQGLEWMGWINPQSGGTNYAQKFQGRVTMTRDTSTSTAYMELSRLRSDDTAVYYCARDHGSRHFWSYWGFDYWGQGTLVTVSS

SEQ ID NO: 35 - protein, 124 aa, Artificial Sequence
  MF6069
QVQLVQSGAEVKKPGASVKVSCKASGYTFTGYYMHWVRQAPGQGLEWMGWINPQSGGTNYAQKFQGRVTMTRDTSISTAYMELSRLRSDDTAVYYCARDHGSRHFWSYWGFDYWGQGTLVTVSS

SEQ ID NO: 36 - protein, 124 aa, Artificial Sequence
  MF6070
QVQLVQSGAEVKKPGASVKVSCKASGYTFTSYYMHWVRQAPGQGLEWMGWINPSGGSTNYAQKFQGRVTMTRDTSTSTVYMELSRLRSEDTAVYYCARDHGSRHFWSYWGFDYWGQGTLVTVSS

SEQ ID NO: 37 - protein, 124 aa, Artificial Sequence
  MF6071
QVQLVQSGAEVKKPGASVKVSCKASGYTFTGYYMHWVRQAPGQGLEWMGWINPSSGSTNYAQKFQGRVTMTRDTSTSTAYMELSSLRSEDTAVYYCARDHGSRHFWSYWGFDYWGQGTLVTVSS

SEQ ID NO: 38 - protein, 124 aa, Artificial Sequence
  MF6072
QVQLVQSGAEVKKPGASVKVSCKASGYTFTGYYMHWVRQAPGQGLEWMGWINPSSGGTNYAQKFQGRVTMTRDTSTSTVYMELSSLRSDDTAVYYCARDHGSRHFWSYWGFDYWGQGTLVTVSS

SEQ ID NO: 39 - protein, 124 aa, Artificial Sequence
  MF6073
QVQLVQSGAEVKKPGASVKVSCKASGYTFTGYYMHWVRQAPGQGLEWMGWINPSSGGTNYAQKFQGRVTMTRDTSTSTAYMELSRLRSDDTAVYYCARDHGSRHFWSYWGFDYWGQGTLVTVSS

SEQ ID NO: 40 - protein, 124 aa, Artificial Sequence
  MF6074
QVQLVQSGAEVKKPGASVKVSCKASGYTFTGYYMHWVRQAPGQGLEWMGWINPSSGGTNYAQKFQGRVTMTRDTSISTAYMELSRLRSDDTAVYYCARDHGSRHFWSYWGFDYWGQGTLVTVSS

SEQ ID NO: 41 - DNA, 370 bp, Artificial Sequence
  MF2926 VH
ggcccagccggccatggcccaggtccagctgcagcagtctggacctgagctggtgaaacctggggcttcagtgatgatttcctgcaaggcttctggttactcattcactggctaccacatgaactgggtgaagcaaagtcctgaaaagagccttgagtggattggagacataaatcctagcattggtacgactgcccacaaccagattttcagggccaaggccacaatgactgttgacaaatcctccaacacagcctacatgcagctcaagagcctgacatctgaagactctggagtcttttactgtgttagaagaggggactggtccttcgatgtctggggcacagggaccacggtcaccgtctccagt

SEQ ID NO: 42 - protein, 117 aa, Artificial Sequence
  MF2926 VH
QVQLQQSGPELVKPGASVMISCKASGYSFTGYHMNWVKQSPEKSLEWIGDINPSIGTTAHNQIFRAKATMTVDKSSNTAYMQLKSLTSEDSGVFYCVRRGDWSFDVWGTGTTVTVSS

SEQ ID NO: 43 - protein, 16 aa, Artificial Sequence
  MF2926 VH CDR1
GYHMNWVKQSPEKSLE

SEQ ID NO: 44 - protein, 6 aa, Artificial Sequence
  MF2926 VH CDR2
NQIFRA

SEQ ID NO: 45 - protein, 8 aa, Artificial Sequence
  MF2926 VH CDR3
RGDWSFDV

SEQ ID NO: 46 - DNA, 379 bp, Artificial Sequence
  MF2930 VH
ggcccagccggccatggccgaggtccagctgcagcagtctggggctgaactggtgaagcctggagcctcagtgatgatgtcctgtaaggtttctggctacaccttcacttcctatcctatagcgtggatgaagcaggttcatggaaagagcctagagtggattggaaattttcatccttacagtgatgatactaagtacaatgaaaacttcaagggcaaggccacattgactgtagaaaaatcctctagcacagtctacttggagctcagccgattaacatctgatgactctgctgtttattactgtgcaagaagtaacccattatattactttgctatggactactggggtcaaggaacctcggtcaccgtctccagt

SEQ ID NO: 47 - protein, 120 aa, Artificial Sequence
  MF2930 VH
EVQLQQSGAELVKPGASVMMSCKVSGYTFTSYPIAWMKQVHGKSLEWIGNFHPYSDDTKYNENFKGKATLTVEKSSSTVYLELSRLTSDDSAVYYCARSNPLYYFAMDYWGQGTSVTVSS

SEQ ID NO: 48 - protein, 16 aa, Artificial Sequence
  MF2930 VH CDR1
SYPIAWMKQVHGKSLE

SEQ ID NO: 49 - protein, 6 aa, Artificial Sequence
  MF2930 VH CDR2
NENFKG

SEQ ID NO: 50 - protein, 11 aa, Artificial Sequence
  MF2930 VH CDR3
SNPLYYFAMDY

SEQ ID NO: 51 - DNA, 385 bp, Artificial Sequence
  MF1849 VH
ggcccagccggccatggcccaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaaggtgactacggttcttactcttcttacgcctttgattattggggccaaggtaccctggtcaccgtctccagt

SEQ ID NO: 52 - protein, 122 aa, Artificial Sequence
  MF1849 VH
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGDYGSYSSYAFDYWGQGTLVTVSS

SEQ ID NO: 53 - protein, 5 aa, Artificial Sequence
  MF1849 VH CDR1
SYGMH

SEQ ID NO: 54 - protein, 17 aa, Artificial Sequence
  MF1849 VH CDR2
VISYDGSNKYYADSVKG

SEQ ID NO: 55 - protein, 13 aa, Artificial Sequence
  MF1849 VH CDR3
GDYGSYSSYAFDY

SEQ ID NO: 56 - DNA, 385 bp, Artificial Sequence
  MF2973 VH
ggcccagccggccatggcccaggtgcagctgaagcagtctggggctgagctggtgaggcctggggcttcagtgaagttgtcctgcaaggcttctggctacattttcactggctactatataaactggttgaggcagaggcctggacagggacttgaatggattgcaaaaatttatcctggaagtggtaatacttactacaatgagaagttcaggggcaaggccacactgactgcagaagaatcctccagcactgcctacatgcagctcagcagcctgacatctgaggactctgctgtctatttctgtgcaagagggccccactatgattacgacggcccctggtttgtttactggggccaagggactctggtcaccgtctccagt

SEQ ID NO: 57 - protein, 122 aa, Artificial Sequence
  MF2973 VH
QVQLKQSGAELVRPGASVKLSCKASGYIFTGYYINWLRQRPGQGLEWIAKIYPGSGNTYYNEKFRGKATLTAEESSSTAYMQLSSLTSEDSAVYFCARGPHYDYDGPWFVYWGQGTLVTVSS

SEQ ID NO: 58 - protein, 16 aa, Artificial Sequence
  MF2973 CDR1
GYYINWLRQRPGQGLE

SEQ ID NO: 59 - protein, 6 aa, Artificial Sequence
  MF2973 VH CDR2
NEKFRG

SEQ ID NO: 60 - protein, 13 aa, Artificial Sequence
  MF2973 VH CDR3
GPHYDYDGPWFVY

SEQ ID NO: 61 - DNA, 382 bp, Artificial Sequence
  MF3004 VH
ggcccagccggccatggcccaggtgcagctgaagcagtctggggctgagctggtgaggcctggggcttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccagcactgcctacatgcacctcagcagcctgacatctgaggactctgctgtctatttctgtgcaagaccccactatggttacgacgactggtacttcggtgtctggggcacaggcaccacggtcaccgtctccagt

SEQ ID NO: 62 - protein, 121 aa, Artificial Sequence
  MF3004 VH
QVQLKQSGAELVRPGASVKLSCKASGYTFTGYYINWVKQRPGQGLEWIARIYPGSGYTYYNEKFKGKATLTAEESSSTAYMHLSSLTSEDSAVYFCARPHYGYDDWYFGVWGTGTTVTVSS

SEQ ID NO: 63 - protein, 16 aa, Artificial Sequence
  MF3004 VH CDR1
GYYINWVKQRPGQGLE

SEQ ID NO: 64 - protein, 6 aa, Artificial Sequence
  MF3004 VH CDR2
NEKFKG

SEQ ID NO: 65 - protein, 12 aa, Artificial Sequence
  MF3004 VH CDR3
PHYGYDDWYFGV

SEQ ID NO: 66 - DNA, 382 bp, Artificial Sequence
  MF2971 VH
ggcccagccggccatggcccaggtgcagctgaagcagtctggggctgagctggtgaggcctggggcttcagtgaaactgtcctgcaaggcttctggctacactttcactgcctactatataaactgggtgaagcagaggcctggacagggacttgagtggattgcaaggatttatcctggaagtggctatacttactacaatgagattttcaagggcagggccacactgactgcagacgaatcctccagcactgcctacatgcaactcagcagcctgacatctgaggactctgctgtctatttctgtgcaagacctccggtctactatgactcggcctggtttgcttactggggccaagggactctggtcaccgtctccagt

SEQ ID NO: 67 - protein, 121 aa, Artificial Sequence
  MF2971 VH
QVQLKQSGAELVRPGASVKLSCKASGYTFTAYYINWVKQRPGQGLEWIARIYPGSGYTYYNEIFKGRATLTADESSSTAYMQLSSLTSEDSAVYFCARPPVYYDSAWFAYWGQGTLVTVSS

SEQ ID NO: 68 - protein, 16 aa, Artificial Sequence
  MF2971 CDR1
AYYINWVKQRPGQGLE

SEQ ID NO: 69 - protein, 6 aa, Artificial Sequence
  MF2971 VH CDR2
NEIFKG

SEQ ID NO: 70 - protein, 12 aa, Artificial Sequence
  MF2971 VH CDR3
PPVYYDSAWFAY

SEQ ID NO: 71 - DNA, 382 bp, Artificial Sequence
  MF3025 VH
ggcccagccggccatggcccaggtgcagctgaagcagtctggggctgagctggtgaggcctgggacttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccaacactgcctatatgcacctcagcagcctgacatctgaggactctgctgtctatttctgtgcaaggccccactatggttacgacgactggtacttcgctgtctggggcacagggaccacggtcaccgtctccagt

SEQ ID NO: 72 - protein, 121 aa, Artificial Sequence
  MF3025 VH
QVQLKQSGAELVRPGTSVKLSCKASGYTFTGYYINWVKQRPGQGLEWIARIYPGSGYTYYNEKFKGKATLTAEESSNTAYMHLSSLTSEDSAVYFCARPHYGYDDWYFAVWGTGTTVTVSS

SEQ ID NO: 73 - protein, 16 aa, Artificial Sequence
  MF3025 VH \CDR1
GYYINWVKQRPGQGLE

SEQ ID NO: 74 - protein, 6 aa, Artificial Sequence
  MF3025 VH CDR2
NEKFKG

SEQ ID NO: 75 - protein, 12 aa, Artificial Sequence
  MF3025 VH CDR3
PHYGYDDWYFAV

SEQ ID NO: 76 - DNA, 385 bp, Artificial Sequence
  MF2916 VH
ggcccagccggccatggcccaggtccagctgcagcagtctggggctgagctggtgaggcctggggcttcagtgaagctgtcctgcaaggcttctggctacactttcactggctactatataaactgggtgaagcagaggcctggacagggacttgagtggattgcaaggatttatcctggcagtggtcatacttcctacaatgagaagttcaagggcaaggccacactgactacagaaaaatcctccagcactgcctacatgcagctcagcagcctgacatctgaggactctgctgtctatttctgtgcaagacctatctactttgattacgcaggggggtacttcgatgtctggggcacaagaacctcggtcaccgtctccagt

SEQ ID NO: 77 - protein, 122 aa, Artificial Sequence
  MF2916 VH
QVQLQQSGAELVRPGASVKLSCKASGYTFTGYYINWVKQRPGQGLEWIARIYPGSGHTSYNEKFKGKATLTTEKSSSTAYMQLSSLTSEDSAVYFCARPIYFDYAGGYFDVWGTRTSVTVSS

SEQ ID NO: 78 - protein, 16 aa, Artificial Sequence
  MF2916 VH CDR1
GYYINWVKQRPGQGLE

SEQ ID NO: 79 - protein, 6 aa, Artificial Sequence
  MF2916 VH CDR2
NEKFKG

SEQ ID NO: 80 - protein, 13 aa, Artificial Sequence
  MF2916 VH CDR3
PIYFDYAGGYFDV

SEQ ID NO: 81 - DNA, 382 bp, Artificial Sequence
  MF3958 VH
ggcccagccggccatggcccaggtgcagctggtgcagtctggcgccgaagtgaagaaacctggcgccagcgtgaagctgagctgcaaggccagcggctacaccttcaccgcctactacatcaactgggtccgacaggccccaggccagggcctggaatggatcggcagaatctaccccggctccggctacaccagctacgcccagaagttccagggcagagccaccctgaccgccgacgagagcaccagcaccgcctacatggaactgagcagcctgcggagcgaggataccgccgtgtacttctgcgccagaccccccgtgtactacgacagcgcttggtttgcctactggggccagggcaccctggtcaccgtctccagt

SEQ ID NO: 82 - protein, 121 aa, Artificial Sequence
  MF3958 VH
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSS

SEQ ID NO: 83 - protein, 5 aa, Artificial Sequence
  MF3958 VH CDR1
AYYIN

SEQ ID NO: 84 - protein, 17 aa, Artificial Sequence
  MF3958 VH CDR2
RIYPGSGYTSYAQKFQG

SEQ ID NO: 85 - protein, 12 aa, Artificial Sequence
  MF3958 VH CDR3
PPVYYDSAWFAY

SEQ ID NO: 86 - DNA, 382 bp, Artificial Sequence
  MF3031 VH
ggcccagccggccatggcccaggtccagctgcagcagtctggggctgagctggtgaggcctggggcttcagtgaagctgtcctgcaaggcttctggctacactttcactgcctactatataaactgggtgaagcagaggcctggacagggacttgagtggattgcaaagatttatcctggaagtggttatacttactacaatgagaatttcaggggcaaggccacactgactgcagaagaatcctccagtactgcctacatacaactcagcagcctgacatctgaggactctgctgtctatttctgtgcaagaggcgtctatgattacgacggggcctggtttgcttactggggccaagggactctggtcaccgtctccagt

SEQ ID NO: 87 - protein, 121 aa, Artificial Sequence
  MF3031 VH
QVQLQQSGAELVRPGASVKLSCKASGYTFTAYYINWVKQRPGQGLEWIAKIYPGSGYTYYNENFRGKATLTAEESSSTAYIQLSSLTSEDSAVYFCARGVYDYDGAWFAYWGQGTLVTVSS

SEQ ID NO: 88 - protein, 16 aa, Artificial Sequence
  MF3031 VH CDR1
AYYINWVKQRPGQGLE

SEQ ID NO: 89 - protein, 6 aa, Artificial Sequence
  MF3031 VH CDR2
NENFRG

SEQ ID NO: 90 - protein, 12 aa, Artificial Sequence
  MF3031 VH CDR3
GVYDYDGAWFAY

SEQ ID NO: 91 - DNA, 382 bp, Artificial Sequence
  MF3991 VH
ggcccagccggccatggcccaggtgcagctggtgcagtctggcgccgaagtgaagaaacctggcgccagcgtgaagctgagctgcaaggccagcggctacaccttcaccgcctactacatcaactgggtccgacaggccccaggccagggcctggaatggatcggcagaatctaccccggctccggctacaccagctacgcccagaagttccagggcagagccaccctgaccgccgacgagagcaccagcaccgcctacatggaactgagcagcctgcggagcgaggataccgccgtgtacttctgcgccagaccccactacggctacgacgactggtacttcggcgtgtggggccagggcaccctggtcaccgtctccagt

SEQ ID NO: 92 - protein, 121 aa, Artificial Sequence
  MF3991 VH
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPHYGYDDWYFGVWGQGTLVTVSS

SEQ ID NO: 93 - protein, 5 aa, Artificial Sequence
  MF3991 VH CDR1
AYYIN

SEQ ID NO: 94 - protein, 17 aa, Artificial Sequence
  MF3991 VH CDR2
RIYPGSGYTSYAQKFQG

SEQ ID NO: 95 - protein, 12 aa, Artificial Sequence
  MF3991 VH CDR3
PHYGYDDWYFGV

SEQ ID NO: 96 - DNA, 391 bp, Artificial Sequence
  MF3178 VH
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 97 - protein, 124 aa, Artificial Sequence; identical to SEQ ID NO: 23
  MF3178 VH

SEQ ID NO: 98 - protein, 5 aa, Artificial Sequence
  MF3178 VH CDR1
GYYMH

SEQ ID NO: 99 - protein, 17 aa, Artificial Sequence
  MF3178 VH CDR2
WINPNSGGTNYAQKFQG

SEQ ID NO: 100 - protein, 15 aa, Artificial Sequence
  MF3178 VH CDR3
DHGSRHFWSYWGFDY

SEQ ID NO: 101 - DNA, 385 bp, Artificial Sequence
  MF3176 VH
ggcccagccggccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggctgtgtattactgtgcaagagattggtggtacccgccgtactactggggctttgattattggggccaaggtaccctggtcaccgtctccagt

SEQ ID NO: 102 - protein, 122 aa, Artificial Sequence
  MF3176 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWWYPPYYWGFDYWGQGTLVTVSS

SEQ ID NO: 103 - protein, 5 aa, Artificial Sequence
  MF3176 VH CDR1
SYAMS

SEQ ID NO: 104 - protein, 17 aa, Artificial Sequence
  MF3176 VH CDR2′
AISGSGGSTYYADSVKG

SEQ ID NO: 105 - protein, 13 aa, Artificial Sequence
  MF3176 VH CDR3
DWWYPPYYWGFDY

SEQ ID NO: 106 - DNA, 391 bp, Artificial Sequence
  MF3163 VH
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaaaagattcttactctcgtcatttctactcttggtgggcctttgattattggggccaaggtaccctggtcaccgtctccagt

SEQ ID NO: 107 - protein, 124 aa, Artificial Sequence
  MF3163 VH
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAKDSYSRHFYSWWAFDYWGQGTLVTVSS

SEQ ID NO: 108 - protein, 5 aa, Artificial Sequence
  MF3163 VH CDR1
GYYMH

SEQ ID NO: 109 - protein, 17 aa, Artificial Sequence
  MF3163 VH CDR2
WINPNSGGTNYAQKFQG

SEQ ID NO: 110 - protein, 15 aa, Artificial Sequence
  MF3163 VH CDR3
DSYSRHFYSWWAFDY

SEQ ID NO: 111 - DNA, 382 bp, Artificial Sequence
  MF3099 VH
ggcccagccggccatggccgaggtccagctgcagcagcctggggctgagctggtgaggcctgggacttcagtgaagttgtcctgcaaggcttctggctacaccttcaccagctactggatgcactgggtaaagcagaggcctggacaaggccttgagtggatcggaattcttgatccttctgatagttatactacctacaatcaaaagttcaagggcaaggccacattaacagtagacacatcctccagcatagcctacatgcagctcagcagcctgacatctgaggactctgcgctctattactgtgcaagagggggagattacgacgagggaggtgctatggactactggggtcaaggaacctcggtcaccgtctccagt

SEQ ID NO: 112 - protein, 121 aa, Artificial Sequence
  MF3099 VH
EVQLQQPGAELVRPGTSVKLSCKASGYTFTSYWMHWVKQRPGQGLEWIGILDPSDSYTTYNQKFKGKATLTVDTSSSIAYMQLSSLTSEDSALYYCARGGDYDEGGAMDYWGQGTSVTVSS

SEQ ID NO: 113 - protein, 5 aa, Artificial Sequence
  MF3099 VH CDR1
SYWMH

SEQ ID NO: 114 - protein, 17 aa, Artificial Sequence
  MF3099 VH CDR2
ILDPSDSYTTYNQKFKG

SEQ ID NO: 115 - protein, 12 aa, Artificial Sequence
  MF3099 VH CDR3
GGDYDEGGAMDY

SEQ ID NO: 116 - DNA, 391 bp, Artificial Sequence
  MF3307 VH
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaagaggttctcgtaaacgtctgtctaactacttcaacgcctttgattattggggccaaggtaccctggtcaccgtctccagt

SEQ ID NO: 117 - protein, 124 aa, Artificial Sequence
  MF3307 VH
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSRKRLSNYFNAFDYWGQGTLVTVSS

SEQ ID NO: 118 - protein, 5 aa, Artificial Sequence
  MF3307 VH CDR1
GYYMH

SEQ ID NO: 119 - protein, 17 aa, Artificial Sequence
  MF3307 VH CDR2
WINPNSGGTNYAQKFQG

SEQ ID NO: 120 - protein, 15 aa, Artificial Sequence
  MF3307 VH CDR3
GSRKRLSNYFNAFDY

SEQ ID NO: 121 - protein, 450 aa, Artificial Sequence
  heavy chain for erbB-2 binding
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 453 aa, Artificial Sequence
  heavy chain for erbB-3 binding
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - DNA, 379 bp, Artificial Sequence
  MF2889 VH
ggcccagccggccatggccgaggtccagctgcagcagtctggagctgagctggtaaggcctgggacttcagtgaaggtgtcctgcaaggcttctggatacgccttcactaattatttgatagagtgggtaaagcagaggcctggccagggccttgagtggattggagtgatttatcctgaaggtggtggtactatctacaatgagaagttcaagggcaaggcaacactgactgcagacaaatcctccagcactgcctacatgcagctcagcggcctgacatctgaggactctgcggtctatttctgtgcaagaggagactatgattacaaatatgctatggactactggggtcaaggaacctcggtcaccgtctccagt

SEQ ID NO: 124 - protein, 120 aa, Artificial Sequence
  MF2889 VH
EVQLQQSGAELVRPGTSVKVSCKASGYAFTNYLIEWVKQRPGQGLEWIGVIYPEGGGTIYNEKFKGKATLTADKSSSTAYMQLSGLTSEDSAVYFCARGDYDYKYAMDYWGQGTSVTVSS

SEQ ID NO: 125 - protein, 5 aa, Artificial Sequence
  MF2889 VH CDR1
NYLIE

SEQ ID NO: 126 - protein, 17 aa, Artificial Sequence
  MF2889 VH CDR2
VIYPEGGGTIYNEKFKG

SEQ ID NO: 127 - protein, 11 aa, Artificial Sequence
  MF2889 VH CDR3
GDYDYKYAMDY

SEQ ID NO: 128 - DNA, 370 bp, Artificial Sequence
  MF2913 VH
ggcccagccggccatggccgaggtcaagctgcagcagtctggacctgagctggtgaagcctggcgcttcagtgaagatatcctgcaaggcttctggttactcattcactgactacaaaatggactgggtgaagcagagccatggaaagagcctcgaatggattggaaatattaatcctaacagtggtggtgttatctacaaccagaagttcaggggcaaggtcacattgactgttgacaggtcctccagcgcagcctacatggagctccgcagcctgacatctgaggacactgcagtctattattgttcaagaggactgtgggatgctatggactcctggggtcaaggaacctcggtcaccgtctccagt

SEQ ID NO: 129 - protein, 117 aa, Artificial Sequence
  MF2913 VH
EVKLQQSGPELVKPGASVKISCKASGYSFTDYKMDWVKQSHGKSLEWIGNINPNSGGVIYNQKFRGKVTLTVDRSSSAAYMELRSLTSEDTAVYYCSRGLWDAMDSWGQGTSVTVSS

SEQ ID NO: 130 - protein, 16 aa, Artificial Sequence
  MF2913 VH CDR1
DYKMDWVKQSHGKSLE

SEQ ID NO: 131 - protein, 6 aa, Artificial Sequence
  MF2913 VH CDR2
NQKFRG

SEQ ID NO: 132 - protein, 8 aa, Artificial Sequence
  MF2913 VH CDR3
GLWDAMDS

SEQ ID NO: 133 - DNA, 382 bp, Artificial Sequence
  MF1847 VH
ggcccagccggccatggcccaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaaggttggtggcatccgctgctgtctggctttgattattggggccaaggtaccctggtcaccgtctccagt

SEQ ID NO: 134 - protein, 121 aa, Artificial Sequence
  MF1847 VH
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGWWHPLLSGFDYWGQGTLVTVSS

SEQ ID NO: 135 - protein, 5 aa, Artificial Sequence
  MF1847 VH CDR1
SYGMH

SEQ ID NO: 136 - protein, 17 aa, Artificial Sequence
  MF1847 VH CDR2
VISYDGSNKYYADSVKG

SEQ ID NO: 137 - protein, 12 aa, Artificial Sequence
  MF1847 VH CDR3
GWWHPLLSGFDY

SEQ ID NO: 138 - DNA, 370 bp, Artificial Sequence
  MF3001 VH
ggcccagccggccatggccgaggtccagctgcagcagtctggggctgaactggcaaaacctggggcctcagtgaagctgtcctgcaagacttctggctacaactttcctatctactggatgcactgggtaaaacagaggcctggacggggtctggaatggattggatacattaatcctagtactggttatattaagaacaatcagaagttcaaggacaaggccaccttgactgcagacaaatcctccaacacagcctacatgcagctgaacagcctgacatatgaggactctgcagtctattactgtacaagagaagggataactgggtttacttactggggccaagggactctggtcaccgtctccagt

SEQ ID NO: 139 - protein, 117 aa, Artificial Sequence
  MF3001 VH
EVQLQQSGAELAKPGASVKLSCKTSGYNFPIYWMHWVKQRPGRGLEWIGYINPSTGYIKNNQKFKDKATLTADKSSNTAYMQLNSLTYEDSAVYYCTREGITGFTYWGQGTLVTVSS

SEQ ID NO: 140 - protein, 16 aa, Artificial Sequence
  MF3001 VH CDR1
IYWMHWVKQRPGRGLE

SEQ ID NO: 141 - protein, 6 aa, Artificial Sequence
  MF3001 VH CDR2
NQKFKD

SEQ ID NO: 142 - protein, 8 aa, Artificial Sequence
  MF3001 VH CDR3
EGITGFTY

SEQ ID NO: 143 - DNA, 385 bp, Artificial Sequence
  MF1898 VH
ggcccagccggccatggcccaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaagatggtttccgtcgtactactctgtctggctttgattattggggccaaggtaccctggtcaccgtctccagt

SEQ ID NO: 144 - protein, 122 aa, Artificial Sequence
  MF1898 VH
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDGFRRTTLSGFDYWGQGTLVTVSS

SEQ ID NO: 145 - protein, 5 aa, Artificial Sequence
  MF1898 VH CDR1
SYGMH

SEQ ID NO: 146 - protein, 17 aa, Artificial Sequence
  MF1898 VH CDR2
VISYDGSNKYYADSVKG

SEQ ID NO: 147 - protein, 13 aa, Artificial Sequence
  MF1898 VH CDR3
DGFRRTTLSGFDY

SEQ ID NO: 148 - DNA, 379 bp, Artificial Sequence
  MF3003 VH
ggcccagccggccatggcccaggtgcagctgaagcagtctggacctgagctggtgaagcctggggcctcagtgaagatttcctgcaaggcttctggcgacgcattcagttactcctggatgaactgggtgaagcagaggcctggaaagggtcttgagtggattggacggatttatcctggagatggagatattaactacaatgggaagttcaagggcaaggccacactgactgcagacaaatcctccagcacagcccacctgcaactcaacagcctgacatctgaggactctgcggtctacttctgtgcaagaggacagctcggactagaggcctggtttgcttattggggccaggggactctggtcaccgtctccagt

SEQ ID NO: 149 - protein, 120 aa, Artificial Sequence
  MF3003 VH
QVQLKQSGPELVKPGASVKISCKASGDAFSYSWMNWVKQRPGKGLEWIGRIYPGDGDINYNGKFKGKATLTADKSSSTAHLQLNSLTSEDSAVYFCARGQLGLEAWFAYWGQGTLVTVSS

SEQ ID NO: 150 - protein, 16 aa, Artificial Sequence
  MF3003 VH CDR1
YSWMNWVKQRPGKGLE

SEQ ID NO: 151 - protein, 6 aa, Artificial Sequence
  MF3003 VH CDR2
NGKFKG

SEQ ID NO: 152 - protein, 11 aa, Artificial Sequence
  MF3003 VH CDR3
GQLGLEAWFAY

SEQ ID NO: 153 - DNA, 391 bp, Artificial Sequence
  MF6058 VH
ggcccagccggccatggcccaggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaaccctcaaagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcaccgtctccagt

SEQ ID NO: 154 - protein, 124 aa, Artificial Sequence
  MF6058 VH
QVQLVQSGADVKKPGASVKVTCKASGYTFTGYYMHWVRQAPGQALEWMGWINPQSGGTNYAKKFQGRVSMTRETSTSTAYMQLSRLRSDDTATYYCARDHGSRHFWSYWGFDYWGQGTLVTVSS

SEQ ID NO: 155 - protein, 5 aa, Artificial Sequence
  MF6058 VH CDR1
GYYMH

SEQ ID NO: 156 - protein, 17 aa, Artificial Sequence
  MF6058 VH CDR2
WINPQSGGTNYAKKFQG

SEQ ID NO: 157 - protein, 15 aa, Artificial Sequence
  MF6058 VH CDR3
DHGSRHFWSYWGFDY

SEQ ID NO: 158 - DNA, 391 bp, Artificial Sequence
  MF6061 VH
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttaagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 159 - protein, 124 aa, Artificial Sequence
  MF6061 VH
QVQLVQSGAEVKKPGASVKVSCKASGYTFTGYYMHWVRQAPGQGLEWMGWINPQSGGTNYAQKFKGRVTMTRDTSTSTAYMELSRLRSDDTAVYYCARDHGSRHFWSYWGFDYWGQGTLVTVSS

SEQ ID NO: 160 - protein, 5 aa, Artificial Sequence
  MF6061 VH CDR1
GYYMH

SEQ ID NO: 161 - protein, 17 aa, Artificial Sequence
  MF6061 VH CDR2
WINPQSGGTNYAQKFKG

SEQ ID NO: 162 - protein, 15 aa, Artificial Sequence
  MF6061 VH CDR3
DHGSRHFWSYWGFDY

SEQ ID NO: 163 - DNA, 391 bp, Artificial Sequence
  MF6065 VH
ggcccagccggccatggcccaggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaaccctcaggggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcaccgtctccagt

SEQ ID NO: 164 - protein, 124 aa, Artificial Sequence
  MF6065 VH
QVQLVQSGAEVKKPGASVKVSCKASGYTFTSYYMHWVRQAPGQGLEWMGWINPQGGSTNYAQKFQGRVTMTRDTSTSTVYMELSRLRSEDTAVYYCARDHGSRHFWSYWGFDYWGQGTLVTVSS

SEQ ID NO: 165 - protein, 5 aa, Artificial Sequence
  MF6065 VH CDR1
SYYMH

SEQ ID NO: 166 - protein, 17 aa, Artificial Sequence
  MF6065 VH CDR2
WINPQGGSTNYAQKFQG

SEQ ID NO: 167 - protein, 15 aa, Artificial Sequence
  MF6065 VH CDR3
DHGSRHFWSYWGFDY

SEQ ID NO: 168 - protein, 124 aa, Artificial Sequence
  MF6058
QVQLVQSGADVKKPGASVKVTCKASGYTFTGYYMHWVRQAPGQALEWMGWINPNSGGTNYAQKFQGRVTMTRDTSISTAYMELSRLRSDDTAVYYCARDHGSRHFWSYWGFDYWGQGTLVTVSS

SEQ ID NO: 169 - protein, 124 aa, Artificial Sequence; identical to SEQ ID NO: 159
  MF6061

SEQ ID NO: 170 - protein, 124 aa, Artificial Sequence
  MF6065
QVQLVQSGAEVKKPGASVKVSCKASGYTFTSYYMHWVRQAPGQGLEWMGWINPQGGSTNYAQKFQGRVTMTRDTSTSTAVMELSRLRSEDTAVYYCARDHGSRHFWSYWGFDYWGQGTLVTVSS